(19) United States
(12) Patent Application Publication
(10) Pub. No.: US 2024/0043413 A1
(43) Pub. Date: Feb. 8, 2024

(54) COMPOUNDS AND APPLICATIONS THEREOF IN FIELD OF OPTOELECTRONICS

(71) Applicant: Zhejiang Brilliant Optoelectronic Technology Co.,Ltd., Taizhou (CN)

(72) Inventors: Junyou PAN, Taizhou (CN); Hong HUANG, Taizhou (CN); Jiahui TAN, Taizhou (CN)

(73) Assignee: Zhejiang Brilliant Optoelectronic Technology Co.,Ltd., Taizhou (CN)

(21) Appl. No.: 18/483,370

(22) Filed: Oct. 9, 2023

Related U.S. Application Data
(63) Continuation of application No. PCT/CN2022/085362, filed on Apr. 6, 2022.

(30) Foreign Application Priority Data
Apr. 7, 2021 (CN) 202110370887.3

(51) Int. Cl. C07D 407/14; C07C 15/38; C07D 407/10; C07C 15/28; C07D 307/91; C07D 409/04; C07D 411/04; C07D 263/57; C07D 413/04; C07D 227/04; C07D 401/10; C07D 403/10; C07D 413/10; C07C 211/61; H10K 85/60; H10K 85/20

(52) U.S. Cl. CPC C07D 407/14; C07C 15/38; C07D 407/10; C07C 15/28; C07D 307/91; C07D 409/04; C07D 411/04; C07D 263/57; C07D 413/04; C07D 227/04; C07D 401/10; C07D 403/10; C07D 413/10; C07C 211/61; H10K 85/622; H10K 85/6574; H10K 85/6576; H10K 85/6572; H10K 85/633; H10K 85/20

(57) ABSTRACT

Provided are compounds including a structural unit of one of formulas (1)-(4). Also provided are formulations containing the compounds, at least one organic solvent, and/or the organic resins. Further provided are organic light-emitting devices containing the compounds.

### CROSS-REFERENCE TO RELATED APPLICATIONS

The present application is a continuation of International Application No. PCT/CN2022/085362, filed on Apr. 6, 2022, which claims priority to Chinese Patent Application No. 202110370887.3, filed on Apr. 7, 2021. All of the aforementioned applications are incorporated herein by reference in their entireties.

### TECHNICAL FIELD

The present disclosure relates to the field of organic optoelectronic material and technology, and in particularly to a compound, a formulation, an organic light-emitting device, and the applications thereof in the optoelectronic field.

### BACKGROUND

According to the principles of colorimetry, the narrower the full width at half maximum (FWHM) of the lights perceived by the human eyes is, the higher the color purity, and thus the more vivid the color display would be. Display devices with narrow-FWHM red, green and blue primary light are able to show vivid views with high color gamut and high visual quality.

The current mainstream full-color displays are achieved mainly in two ways. The first method is to actively emit red, green and blue lights, typically such as RGB-OLED display. The current mature technology is to fabricate light-emitting devices with three colors by vacuum evaporation with fine metal masks, which is complex, at high cost and difficult to achieve high-resolution display over 600 ppi. The second method is using color converters to convert the single-color light from the light-emitting devices into different colors, thereby achieving a full-color display. For example, Samsung combines blue OLEDs with red and green quantum dots (QD) films as the color converters. In this case, the fabrication of the light emitting devices is much simpler, and thus higher yield. Furthermore, the manufacture of the color converters can be achieved by different technologies, such as vacuum evaporation, ink-jet printing, transfer printing and photolithography, etc., appliable to a variety of display products with very different resolution requirements from low resolution large-size TV (around only 50 ppi) to high resolution silicon-based micro-display (over 3000 ppi).

The color conversion materials used in current mainstream color converters are mainly inorganic nanocrystals, commonly known as quantum dots, which are nanoparticles of an inorganic semiconductor material (InP, CdSe, CdS, ZnSe, etc.) with a diameter of 2 nm to 8 nm. The small size of these materials leads to quantum confinement effects, resulting in photoluminescent emissions with a specific frequency, which are highly dependent on the particle size. In this sense, the color of their emission can be readily tuned by adjusting the sizes. Limited by the current synthesis and separation technology of quantum dots, the FWHMs of CD-containing quantum dots typically range from 25 nm to 40 nm, which meet the display requirements of NTSC for color purity. Meanwhile, Cd-free quantum dots generally come with larger FWHMs of 35 nm to 75 nm. Since Cd is considered highly hazardous to environment and human health, most countries have prohibited the use of Cd-containing quantum dots to produce electronic products. In addition, because of the not-sufficiency-large extinction coefficient of quantum dots, the rather thick film required for complete color conversion is rather high, typically above 10 μm. This is a great challenge to the mass production process, especially for Samsung's technology of combing blue OLED with red-green quantum dots. In patent application No. CN202110370887.3, the present inventor proposes the host-dopant concept for the color conversion layer, and the organic materials having a high molar extinction coefficient are chosen as the host, which could absorb the light from the light emitting unit and transfer it to the narrow emissive emitter, thereby realizing a thin color conversion layer. However, the organic materials used as the host often have insufficient solubility and suffer a mismatch with the resin.

Therefore, it is necessary to further develop the new host materials with high extinction coefficient and good processing property as color conversion materials, so that to realize high color gamut in display devices.

### SUMMARY

In one aspect, the present disclosure provides a compound comprising a structural unit of one of formulas (1)-(4).

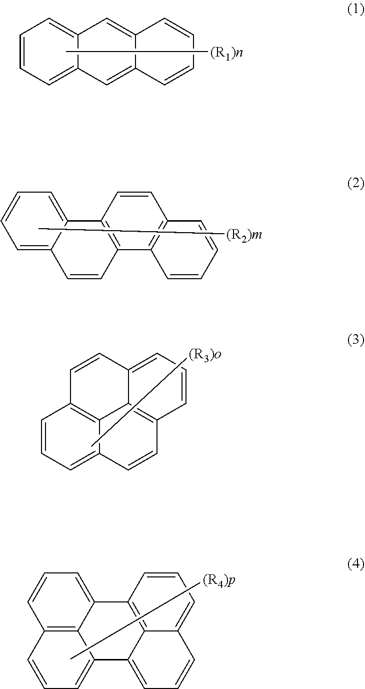

Where R₁ to R₄ are substituents and independently selected from the group consisting of a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group (—CN), a carbamoyl group (—C(═O)NH₂), a haloformyl group (—C(═O)—X where X represents a halogen atom), a formyl group (—C(═O)—H), an isocyano group, an isocyanate group, a thiocyanate group, an isothiocyanate group, a hydroxyl group, a nitro group, —NO₂, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, a disubstituted unit in any position of the above substituents, and any combination thereof;

n, o are integers from 1 to 10; m, p are integers from 1 to 12;

Where the compound comprises at least one cross-linkable group.

In another aspect, the present disclosure also provides a mixture comprising at least one compound as described herein and another functional material, the another functional material is an organic functional material, which can be selected from a hole-injection material (HIM), a hole-transport material (HTM), a hole-blocking material (HBM), an electron-injection material (EIM), an electron-transport material (ETM), an electron-blocking material (EBM), an organic host material (Host), a singlet emitting material (fluorescent emitting material), a triplet emitting material (phosphorescent emitting material), a thermally activated delayed fluorescence material (TADF material), or an organic dye.

In yet another aspect, the present disclosure further provides a formulation comprising at least one compound as described herein, at least one organic solvent, and/or an organic resin.

In yet another aspect, the present disclosure further provides an organic functional film comprising a compound as described herein, or prepared using a formulation as described herein.

In yet another aspect, the present disclosure further provides an optoelectronic device comprising a compound or an organic functional film as described herein.

In yet another aspect, the present disclosure further provides an organic light-emitting device comprising a substrate, a first electrode, an organic light-emitting layer, a second electrode, a color conversion layer, and an encapsulation layer in sequence from bottom to top, the second electrode is at least partially transparent, where 1) the color conversion layer comprises a compound as described herein and an emitter E2; 2) the color conversion layer can at least partially absorbs the light emitted by the organic light-emitting layer through the second electrode; 3) the emission spectrum of the compound is on the short wavelength side of the absorption spectrum of the emitter E2, and at least partially overlaps with the absorption spectrum of the emitter E2; 4) the FWHM of the emission spectrum of the emitter E2 nm.

Beneficial effects: the compound as described herein is well matched with the resin, so that the compound can at least partially participate in the copolymerization or homopolymerization of the resin prepolymer; the compound also has a large solubility, which enable the preparation of green inks for printing or coating processes. Furthermore, the compound with a high extinction coefficient, and the resulting thin color converter are beneficial for achieving a high color gamut display.

### DETAILED DESCRIPTION OF THE EMBODIMENTS

The present disclosure provides a compound, a formulation, an organic light-emitting device, and the applications thereof in the optoelectronic field.

The present disclosure may be embodied in many different forms and is not limited to the embodiments described herein. Rather, these embodiments are provided so that the understanding of the invention of the present disclosure will be more thorough.

Unless otherwise defined, all technical and scientific terms used herein have the same meaning as commonly understood by one of ordinary skill in the art belonging to the present disclosure. The terms used herein in the description of the present disclosure are used only for the purpose of describing specific embodiments and are not intended to be limiting of the present disclosure. As used herein, the term “and/or” includes any and all combinations of one or more of the relevant listed items.

As used herein, the terms “host material”, “matrix material” have the same meaning, and they are interchangeable with each other.

As used herein, the terms “formulation”, “printing ink”, and “ink” have the same meaning, and they are interchangeable with each other.

In one aspect, the present disclosure provides a compound comprising a structural unit of one of the formulas (1)-(4).

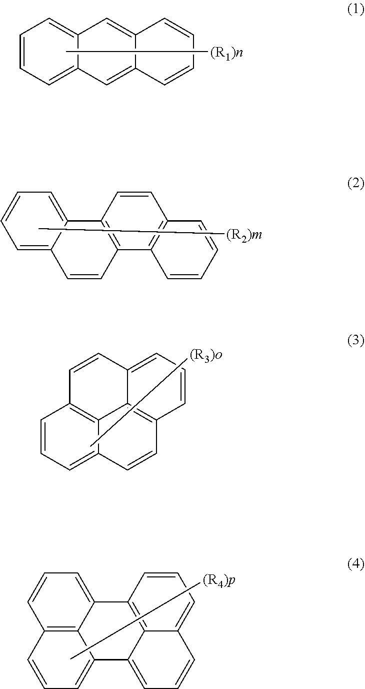

Where R₁ to R₄ are substituents and independently selected from the group consisting of a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group (—CN), a carbamoyl group (—C(═O)NH₂), a haloformyl group (—C(═O)—X where X represents a halogen atom), a formyl group (—C(═O)—H), an isocyano group, an isocyanate group, a thiocyanate group, an isothiocyanate group, a hydroxyl group, a nitro group, —NO₂, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, a disubstituted unit in any position of the above substituents, and any combination thereof; n, o are integers from 1 to 10; m, p are integers from 1 to 12; where the compound comprises at least one cross-linkable group.

In some embodiments, one or more R₁-R₄ may form a monocyclic or polycyclic aliphatic or aromatic ring system with each other and/or with the rings bonded thereto.

In some embodiments, none of R₁-R₄ form a monocyclic or polycyclic aliphatic or aromatic ring system with each other and/or with the rings bonded thereto.

Preferably, R₁ to R₄ are independently selected from the group consisting of a C₁-C₁₀ linear alkyl group, a C₁-C₁₀ linear haloalkyl group, a C₁-C₁₀ linear alkoxy group, a C₁-C₁₀ linear thioalkoxy group, a C₃-C₁₀ branched/cyclic alkyl group, a C₃-C₁₀ branched/cyclic haloalkyl group, a C₃-C₁₀ branched/cyclic alkoxy group, a C₃-C₁₀ branched/cyclic thioalkoxy group, a C₃-C₁₀ branched/cyclic silyl group, a C₁-C₁₀ substituted ketone group, a C₂-C₁₀ alkoxycarbonyl group, a C₇-C₁₀ aryloxycarbonyl group, a cyano group (—CN), a carbamoyl group (—C(═O)NH₂), a haloformyl group (—C(═O)—X where X represents a halogen atom), a formyl group (—C(═O)—H), an isocyano group, an isocyanate group, a thiocyanate group, an isothiocyanate group, a hydroxyl group, a nitro group, —NO₂, —CF₃, —Cl, —Br, —F, —I, a cross-linkable group, a substituted/unsubstituted aromatic or heteroaromatic group containing 5 to 20 ring atoms, an aryloxy or heteroaryloxy group containing 5 to 20 ring atoms, an arylamine or heteroarylamine group containing 5 to 20 ring atoms, a disubstituted unit in any position of the above substituents, and any combination thereof.

In some embodiments, the compound comprises at least two cross-linkable groups.

In some embodiments, the compound comprises at least three cross-linkable groups.

In some embodiments, the compound comprises a structural unit of formulas (1a)-(4a), (4b):

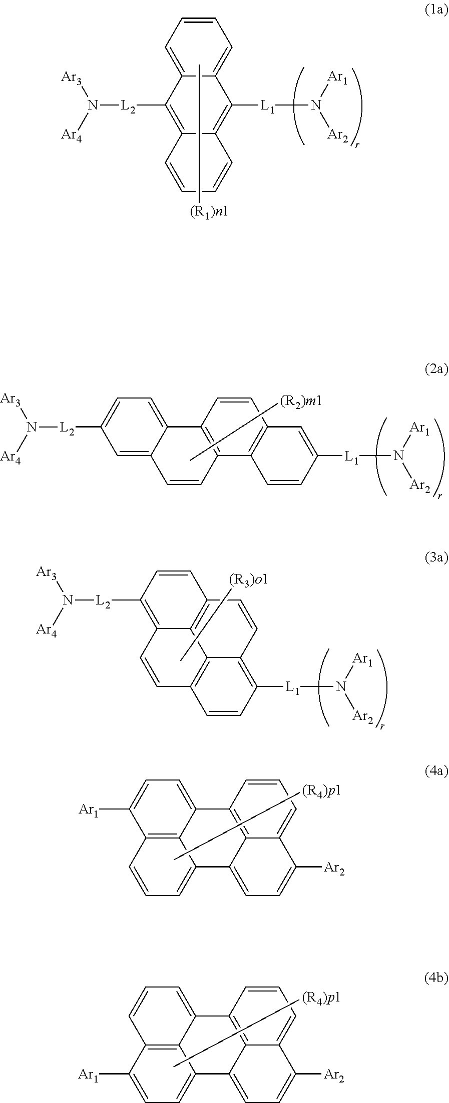

Where n1, of are integers from 1 to 8; m1, p1 are integers from 1 to 10; r is 0 or 1; R₁ to R₄ are identically defined as described above; each of Ar₁ to Ar₄ at each occurrence is independently selected from the group consisting of a substituted/unsubstituted aromatic or heteroaromatic group containing 5 to 40 ring atoms, an aryloxy or heteroaryloxy group containing 5 to 40 ring atoms, and any combination thereof; each of L₁ and L₂ at each occurrence is independently selected from the group consisting of a single bond, a substituted/unsubstituted aromatic or heteroaromatic group containing 6 to 30 ring atoms.

For the purposes of the present disclosure, the aromatic ring system contains 5 to 10 carbon atoms in the ring system, the heteroaromatic ring system contains 1 to 10 carbon atoms and at least one heteroatom in the ring system, provided that the total number of carbon atoms and heteroatoms is at least 4. The heteroatoms are preferably selected from Si, N, P, O, S and/or Ge, particularly preferably selected from Si, N, P, O and/or S. For the purposes of the present disclosure, the aromatic or heteroaromatic ring systems contain not only aromatic or heteroaromatic groups, but also have a plurality of aryl or heteroaryl groups linked by short non-aromatic units (<10% of non-H atoms, preferably <5% of non-H atoms, such as C, N or O atoms). Therefore, a system such as 9,9′-spirobifluorene, 9,9-diarylfluorene, triarylamine, diaryl ether, and the like is also considered to be aromatic ring systems for the purposes of this disclosure.

For the purposes of the present disclosure, the any H atom on the compound may be optionally substituted with R¹. R¹ is defined as the above-mentioned R₁, which may be preferably selected from: (1) a C₁-C₁₀ alkyl group, particularly preferably selected from the following groups: methyl, ethyl, n-propyl, isopropyl, cyclopropyl, n-butyl, isobutyl, sec-butyl, t-butyl, cyclobutyl, 2-methylbutyl, n-pentyl, n-hexyl, cyclohexyl, n-heptyl, cycloheptyl, n-octyl, cyclooctyl, 2-methylheptyl, trifluoromethyl, pentafluoroethyl, 2,2,2-trifluoroethyl, vinyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, or octenyl; (2) a C₁-C₁₀ alkoxy group, particularly preferably methoxy, ethoxy, n-propoxy, isopropoxy, n-butoxy, isobutoxy, sec-butoxy, tert-butoxy, or 2-methylbutoxy; (3) a C₂-C₁₀ aryl or heteroaryl group, which may be monovalent or divalent depending on the application, and in each case can also be optionally substituted with the group R¹ mentioned above and may be attached to an aromatic or heteroaromatic ring at any desired position, particularly preferably selected from the following: benzene, naphthalene, anthracene, dihydropyrene, chrysene, pyrene, fluoranthene, naphthacene, pentacene, benzopyrene, furan, benzofuran, isobenzofuran, dibenzofuran, thiophene, benzothiophene, isobenzothiophene, thiofluorene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenimidazole, pyridimidazole, pyrazine-imidazole, quinoxaline-imidazole, oxazole, benzoxazole, naphthoxazole, anthracenazole, phenoxazole, isoxazole, 1,2-thiazole, 1,3-thiazole, benzothiazole, pyridazine, benzopyridazine, pyrimidine, benzopyrimidine, quinoxaline, pyrazine, phenazine, 1,5-naphthyridine, azocarbazole, benzocholine, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thinadiazole, 1,2,4-thiadiazole, 1,2,5-thiadiazole, 1,3,4-thiadiazole, 1,3,5-triazine, 1,2,4-triazine, 1,2,3-triazine, tetrazole. 1,2,4,5-tetrazine, 1,2,3,4-tetrazine, 1,2,3,5-tetrazine, purine, pteridine, indolizine, or benzothiadiazole. For the purposes of the present disclosure, aromatic and heteroaromatic ring systems are particularly considered to be, in addition to the above-mentioned aryl and heteroaryl groups, also refer to biphenylene, terphenylene, fluorene, spirobifluorene, dihydrophenanthrene, tetrahydropyrene, cis-indenofluorene, or trans-indenofluorene.

In some embodiments, the compound of formulas (1a)-(4a), or (4b), where Ar₁ to Ar₄ are the same or different at each occurrence and are independently selected from the group consisting of an aromatic or heteroaromatic group with 5 to 20, preferably 5 to 18, more preferably 5 to 15, and most preferably 5 to 10 ring atoms; they may be unsubstituted or further substituted with one or two R¹ s. The preferred aromatic or heteraromatic group includes benzene, naphthalene, anthracene, phenanthrene, pyridine, pyrene, or thiophene.

In some embodiments, Ar₁ to Ar₄ are each independently selected from the following structural formulas:

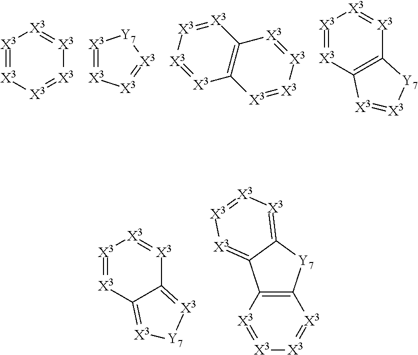

Where each X³ is CR⁶ or N; each Y₇ is independently selected from CR⁷R⁸, SiR⁷R⁸, NR⁶, C(═O), S, or O; R⁶, R⁷, R⁸ are identically defined as the above-mentioned R₁;

Further, each of Ar₁ to Ar₄ may be independently selected from one of the following structural formulas or any combination thereof, which can be further substituted arbitrarily:

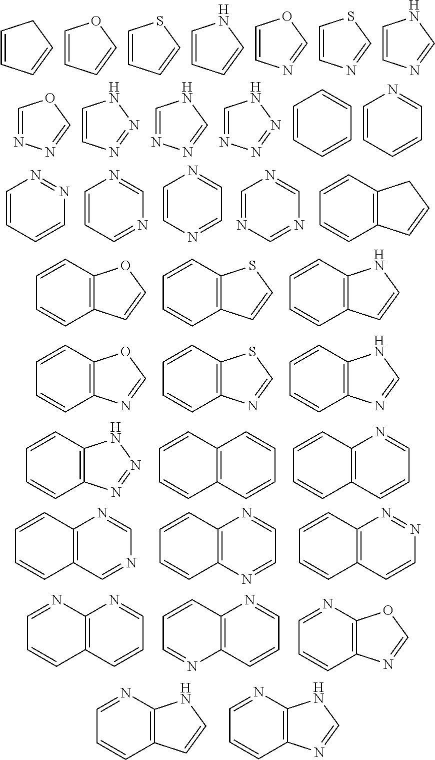

For the purposes of the present disclosure, in some embodiments, each of Ar₁ to Ar₄ is independently selected from benzene, naphthalene, anthracene, phenanthrene, pyridine, pyrene, or thiophene.

In some embodiments, each of L₁ and L₂ is independently selected from a single bond, one of the following groups, or any combination thereof:

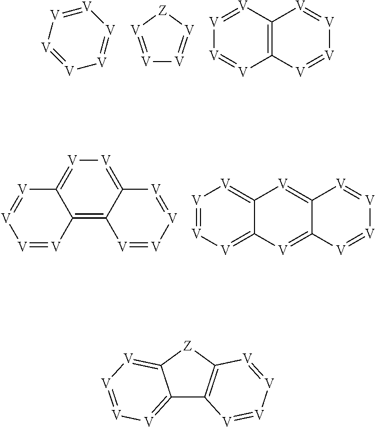

Where each V at each occurrence is CR₁₄ or N; each Z at each occurrence is independently selected from NR₁₅, CR₁₆R₁₇, O, S, SiR₁₈R₁₉, S═O, or SO₂; R₁₄ to R₁₉ at each occurrence are independently selected from: —H, -D, a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, a cross-linkable group, a substituted/unsubstituted aromatic or heteroaromatic group containing 5 to 60 ring atoms, an aryloxy or heteroaryloxy group containing 5 to 60 ring atoms, or any combination thereof.

In some embodiments, each of L₁ and L₂ is independently selected from a single bond, one of the following groups, or any combination thereof:

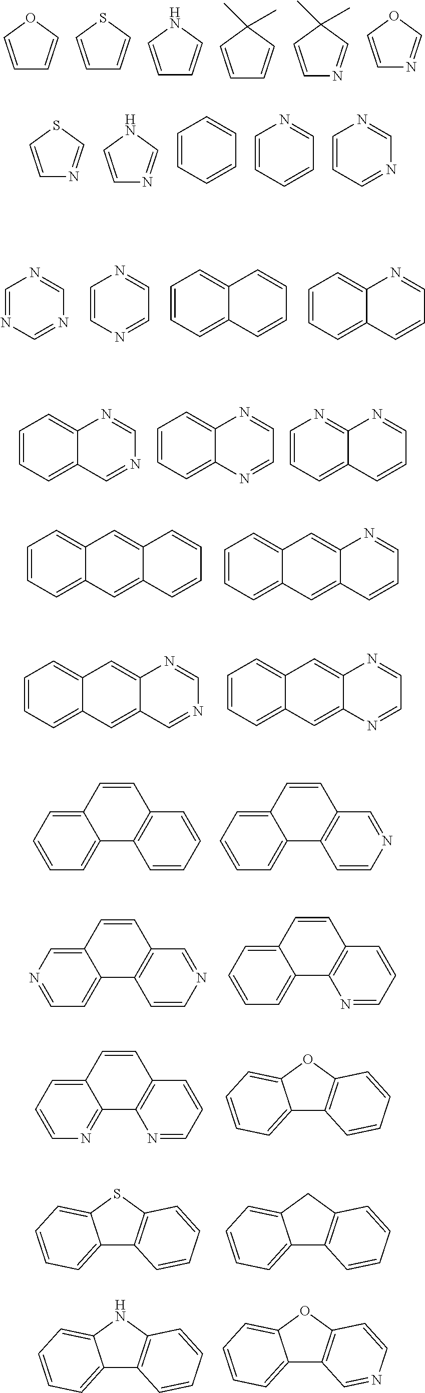

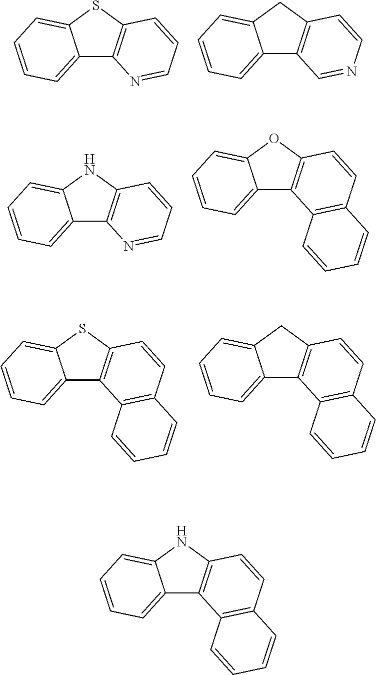

Where H atoms on the ring may be further substituted.

In some embodiments, in the structural unit of formulas (1)-(4), where R₁ to R₄ may be same or different in multiple occurrences, comprising the following structural units or any combination thereof.

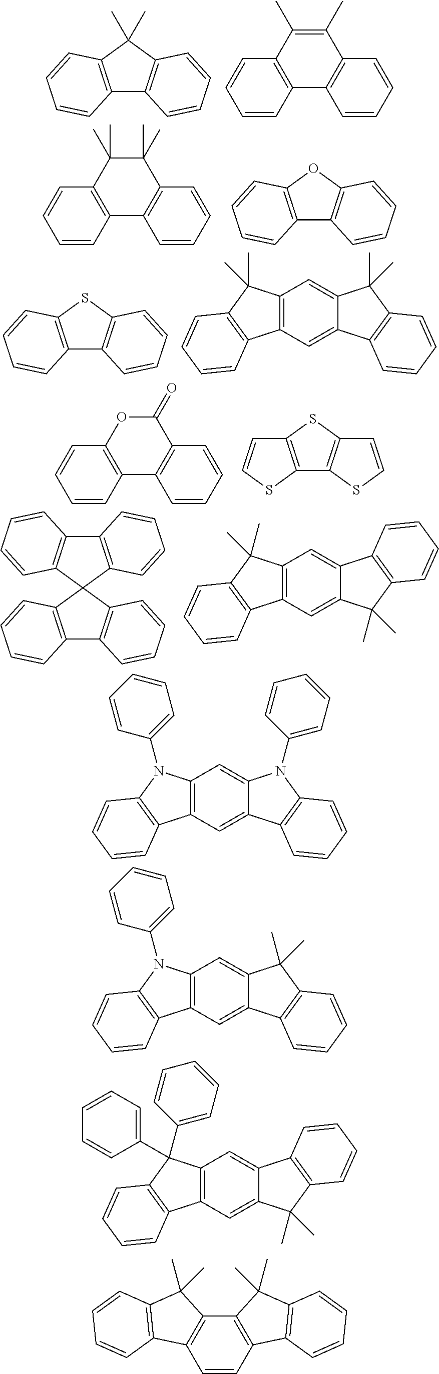

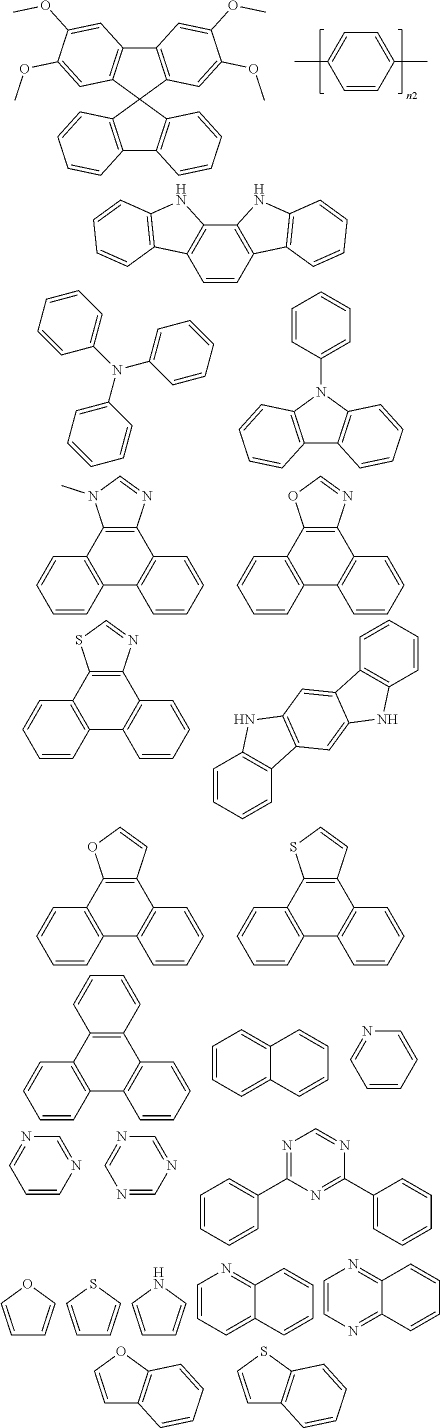

Where n2 is 1, or 2, or 3, or 4.

In some embodiments, in the compound as described herein, at least one of the cross-linkable group is selected from: 1) a linear/cyclic alkenyl, a linear dienyl, a linear alkynyl; 2) an enoxy, a dienoxy; 3) an acrylic; 4) a propylene oxide, an ethylene oxide; 5) a silanyl; 6) a cyclobutanyl.

Preferably, at least one of the cross-linkable group is selected from the following structures:

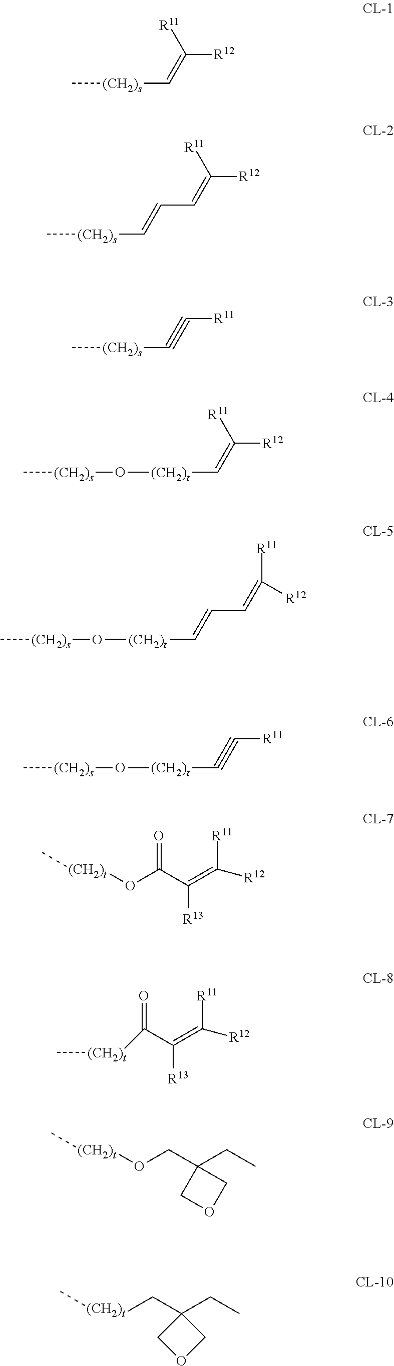

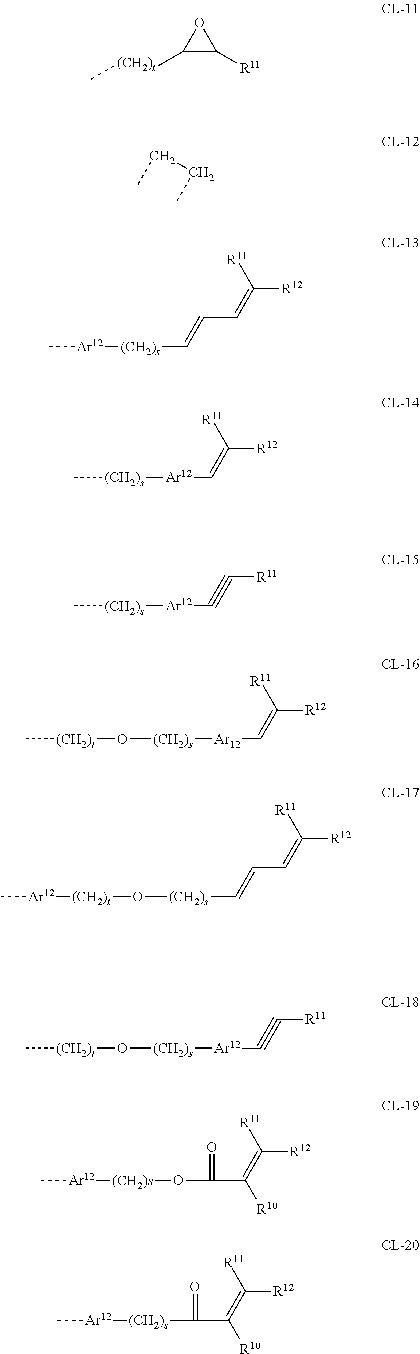

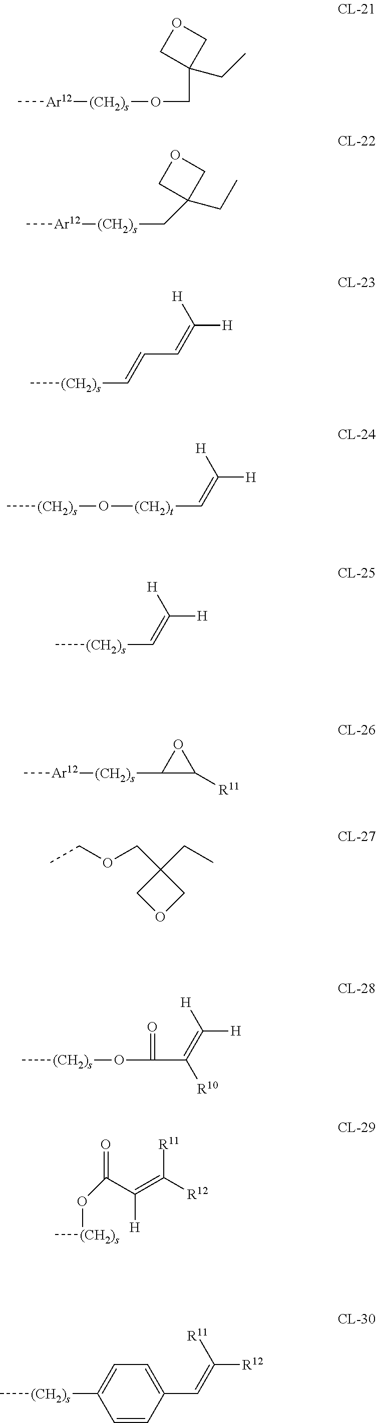

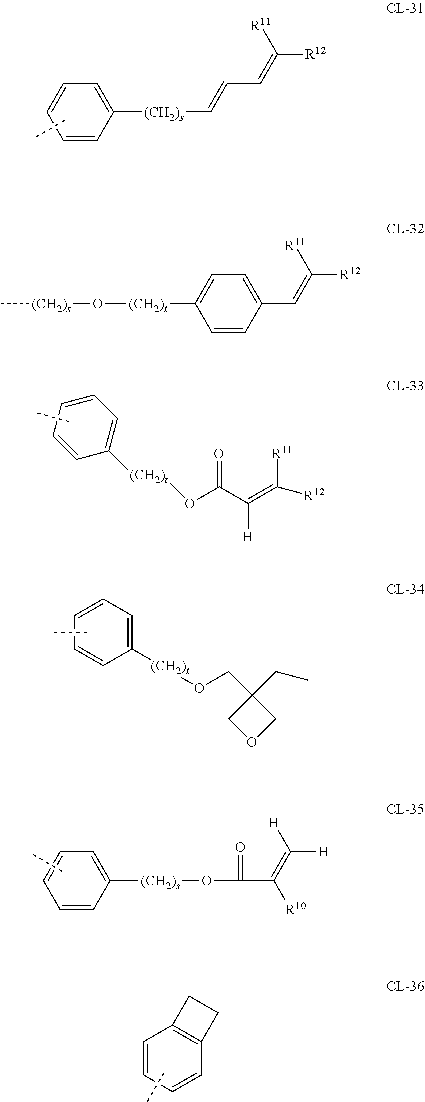

where the dotted line represents a bonded bond, R¹⁰ to R¹³ are identically defined as the above-mentioned R₁; Ar¹² is defined as the above-mentioned Ar₁; s, t at each occurrence are integers >0.

In some embodiments, the cross-linkable structural unit is selected from the following structural formulas:

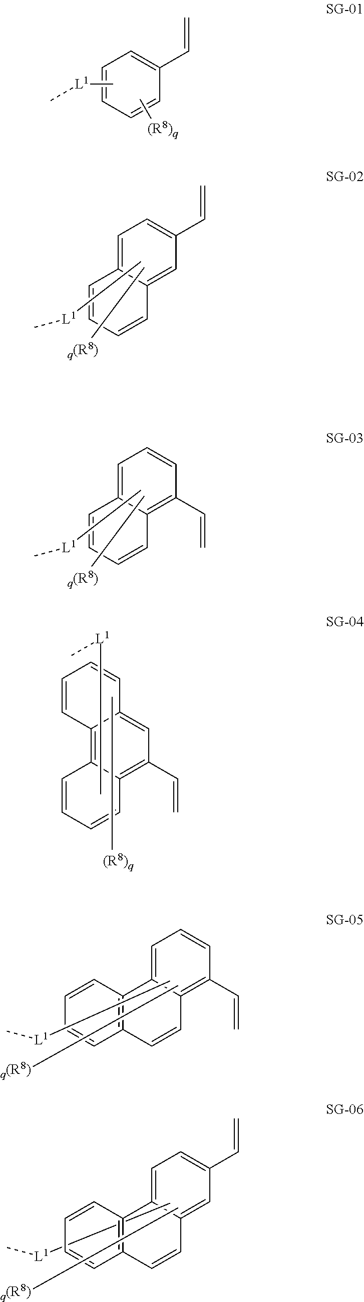

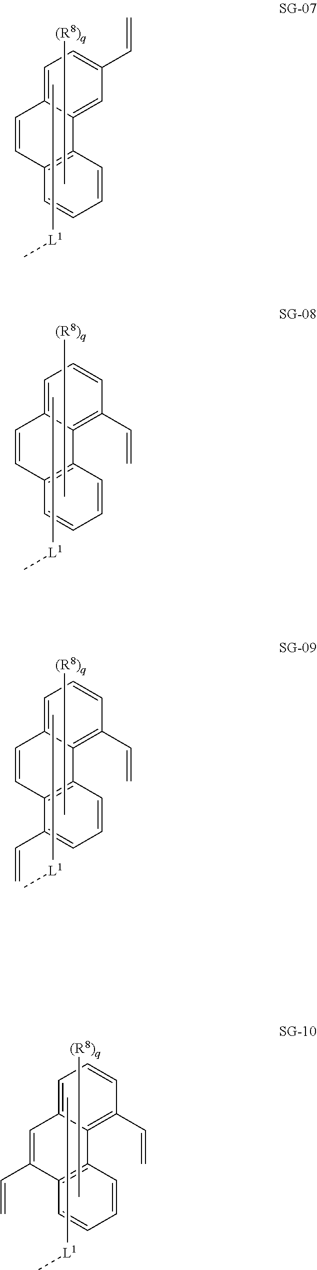

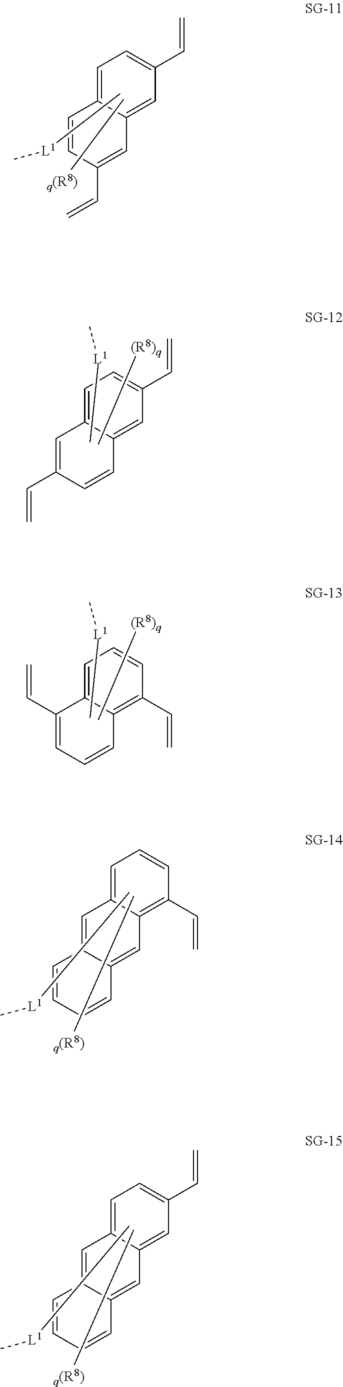

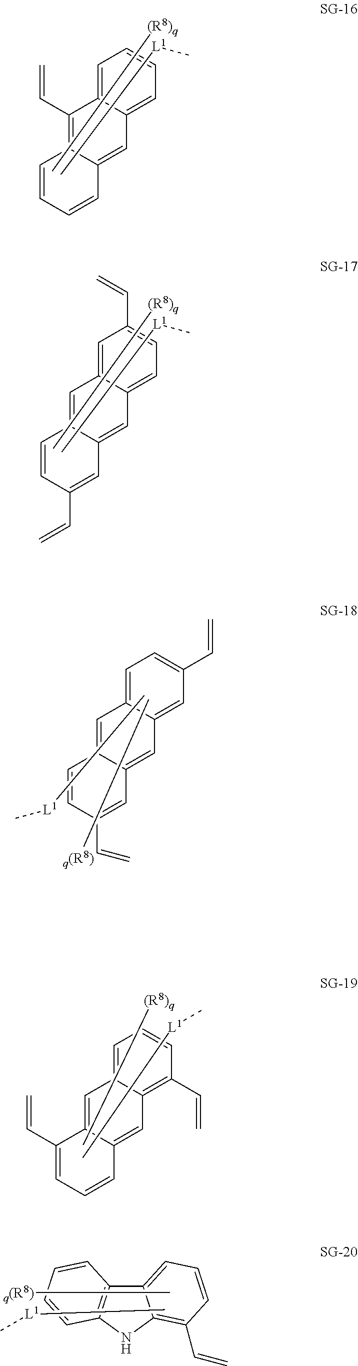

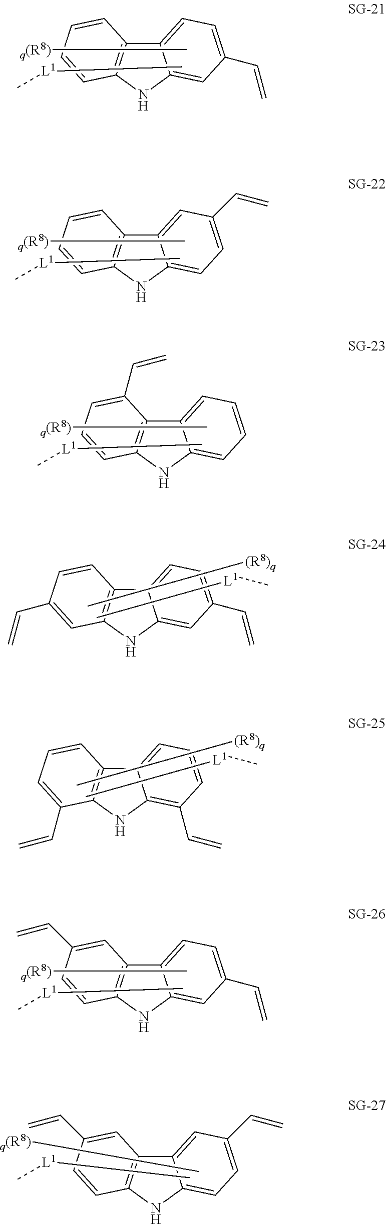

Where R⁸ is defined as described above; q is an integer >0; L¹ represents a single bond or a linking group, and when representing a linking group, it is an aryl or a heteroaryl group; the dotted line represents a bonded bond.

Particularly, L¹ is preferably selected from the following structures:

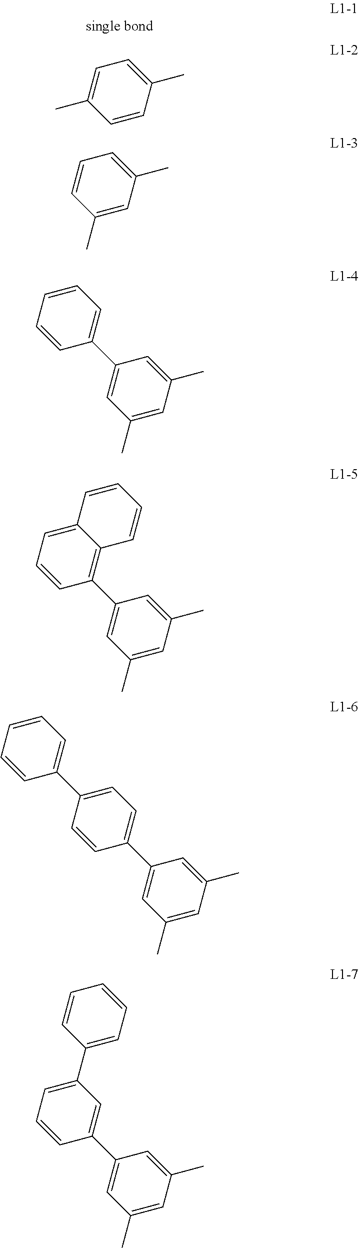

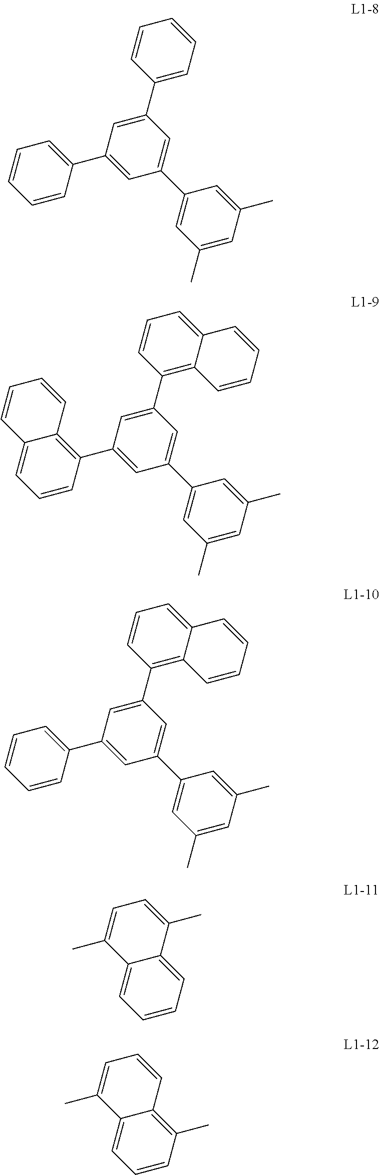

Furthermore, the individual H atoms or CH₂ groups of the present invention can be substituted with the above-mentioned groups or R, R is selected from C₁-C₄₀ alkyl groups, preferably selected from the group consisting of methyl, ethyl, n-propyl, isopropyl, cyclopropyl, n-butyl, isobutyl, sec-butyl, t-butyl, cyclobutyl, methylbutyl, n-pentyl, sec-pentyl, cyclopentyl, n-hexyl, cyclohexyl, n-heptyl, cycloheptyl, n-octyl, cyclooctyl, ethylhexyl, trifluoromethyl, pentafluoroethyl, trifluoroethyl, vinyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl, and octenyl; C₁-C₄₀ alkoxy groups, such as methoxy, trifluoromethoxy, ethoxy, n-propoxy, isopropoxy, n-butoxy, isobutoxy, sec-butoxy, tert-butoxy, or methylbutoxy.

In some embodiments, in the compound as used herein, the total percentage of the SP³ hybrid groups does not exceed 50% of the total molecular weight, more preferably does not exceed 30%, and most preferably does not exceed 20%. The presence of less SP³ hybrid groups can effectively ensure the electrical stability of the compound, thereby ensuring the stability of the devices.

In some embodiments, in order to improve solubility and/or film-forming property, in the compound as used herein, the total percentage of the SP³ hybrid groups exceeds 20% of the total molecular, preferably exceeds 30%, more preferably exceeds 40%, and most preferably exceeds 50%.

In some embodiments, the compound has a high extinction coefficient. The extinction coefficient is also known as the molar extinction coefficient, which refers to the absorption coefficient at a concentration of 1 mol/L, and is represented by the symbol ε, in unit of Lmol⁻¹ cm⁻¹. The extinction coefficient (ε) preferably ≥1*10³; more preferably ≥1*10⁴; particularly preferably ≥5*10⁴; and most preferably ≥1*10⁵. Preferably, the extinction coefficient refers to the extinction coefficient at the wavelength corresponding to the absorption peak.

In some embodiments, the compound has high fluorescence luminescence efficiency, and the photoluminescence quantum efficiency (PLQY) thereof ≥60%, preferably ≥65%, more preferably ≥70%, further preferably ≥80%, and most preferably ≥90%.

Examples of suitable compounds as used herein are listed below, but not limited to:

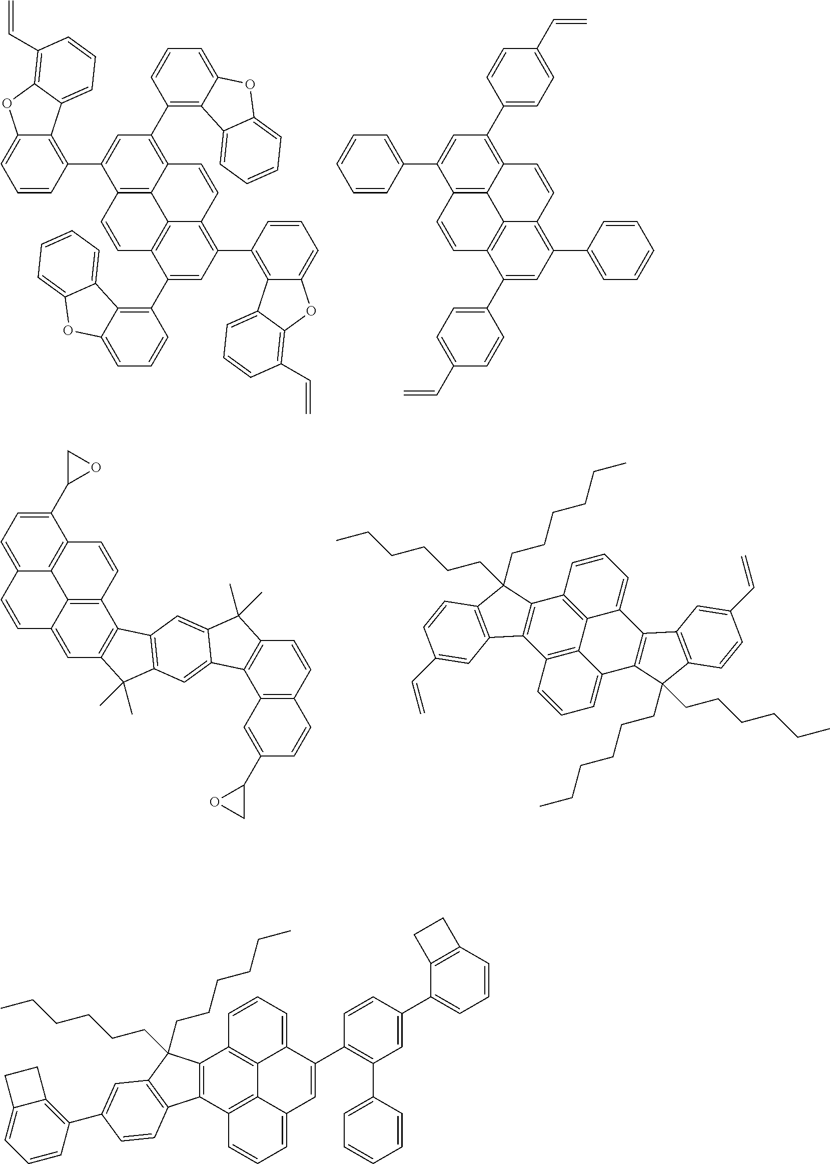

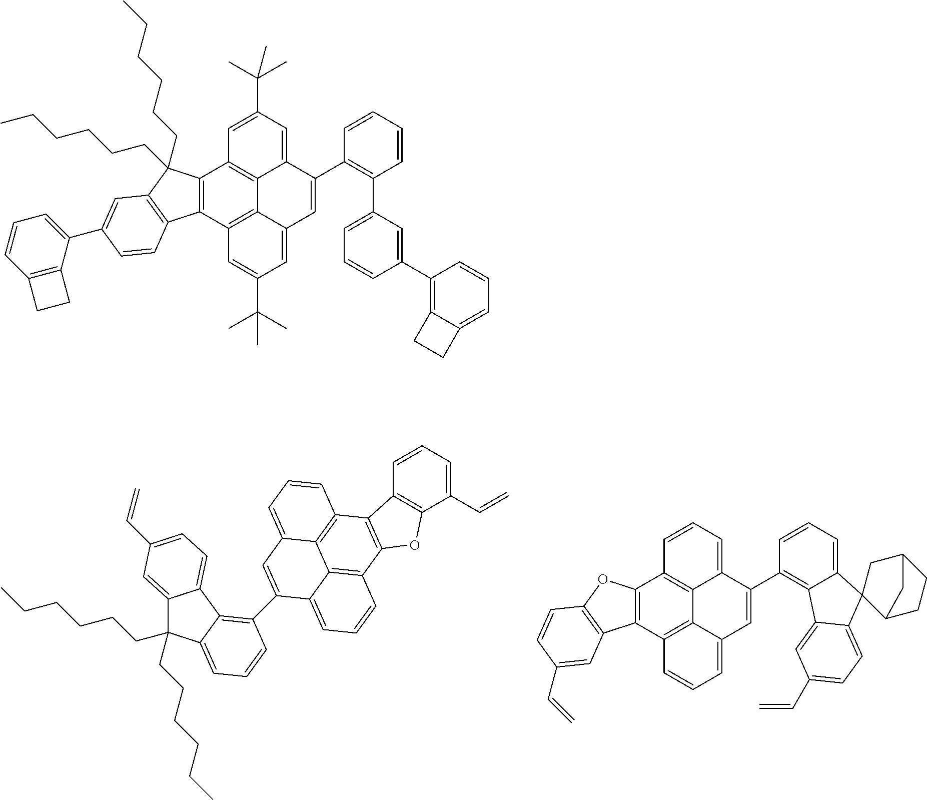

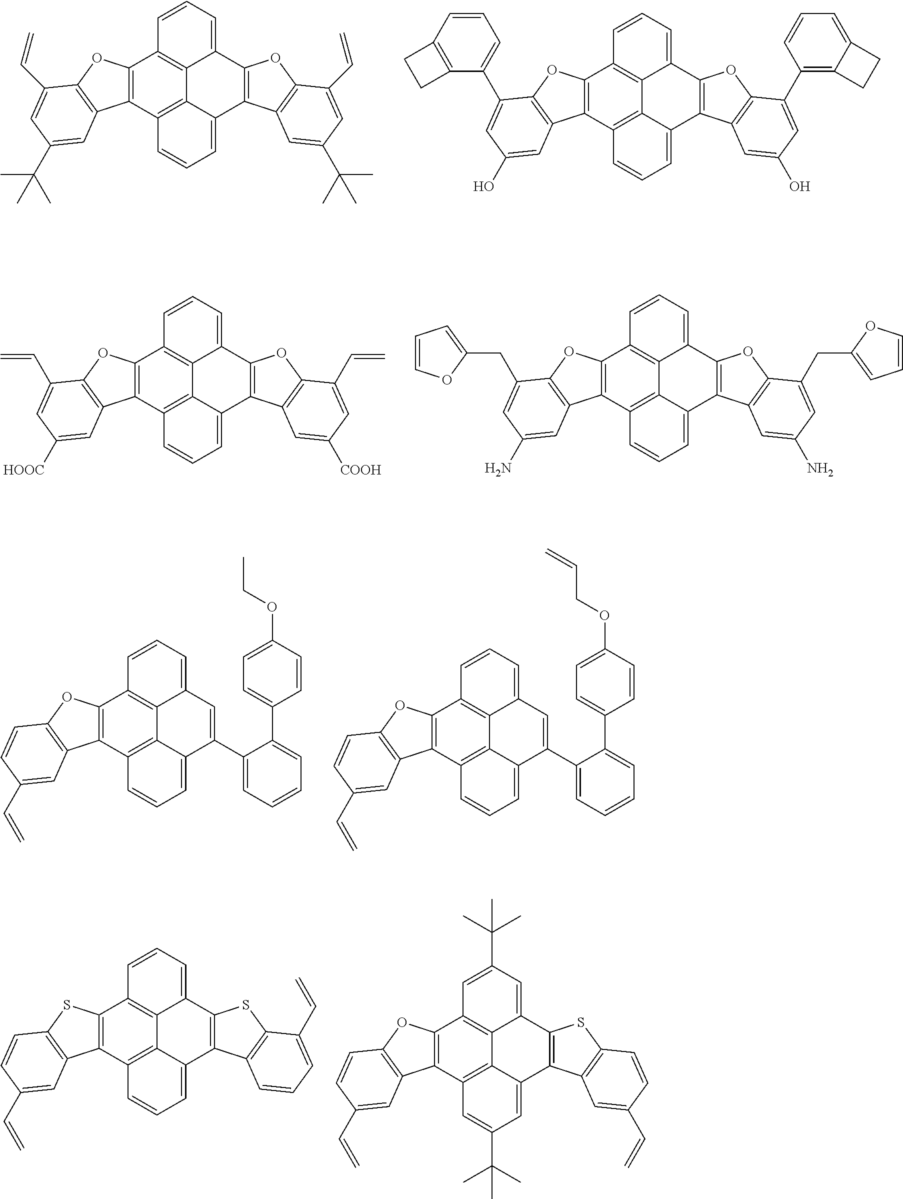

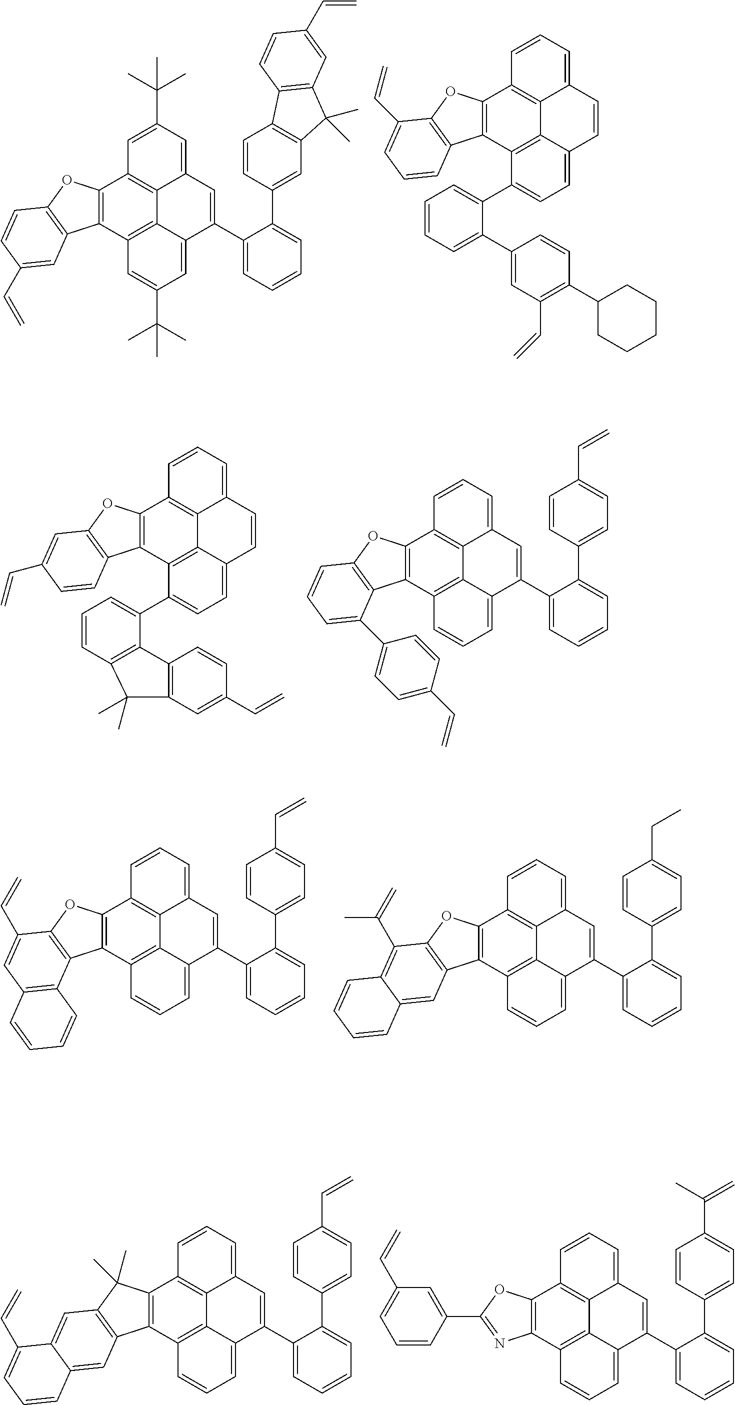

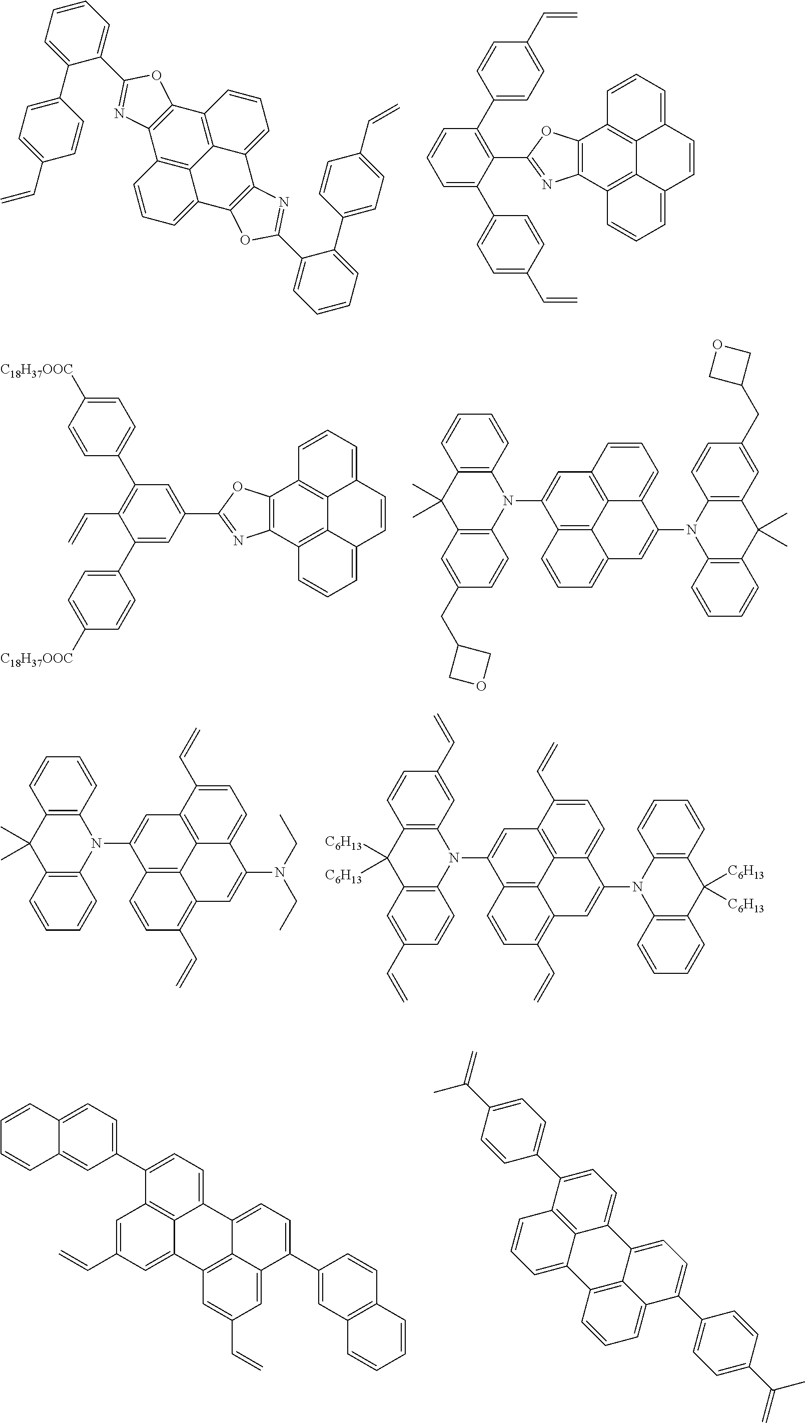

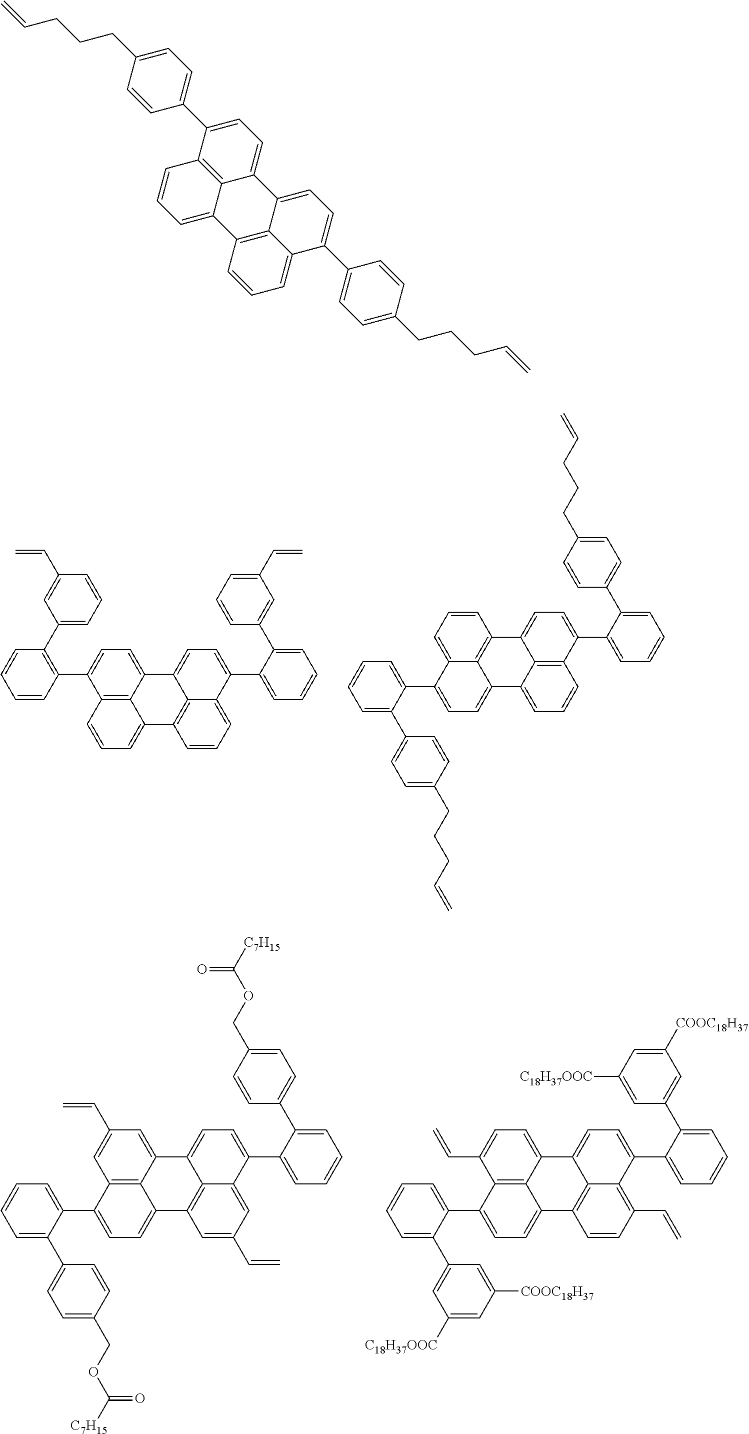

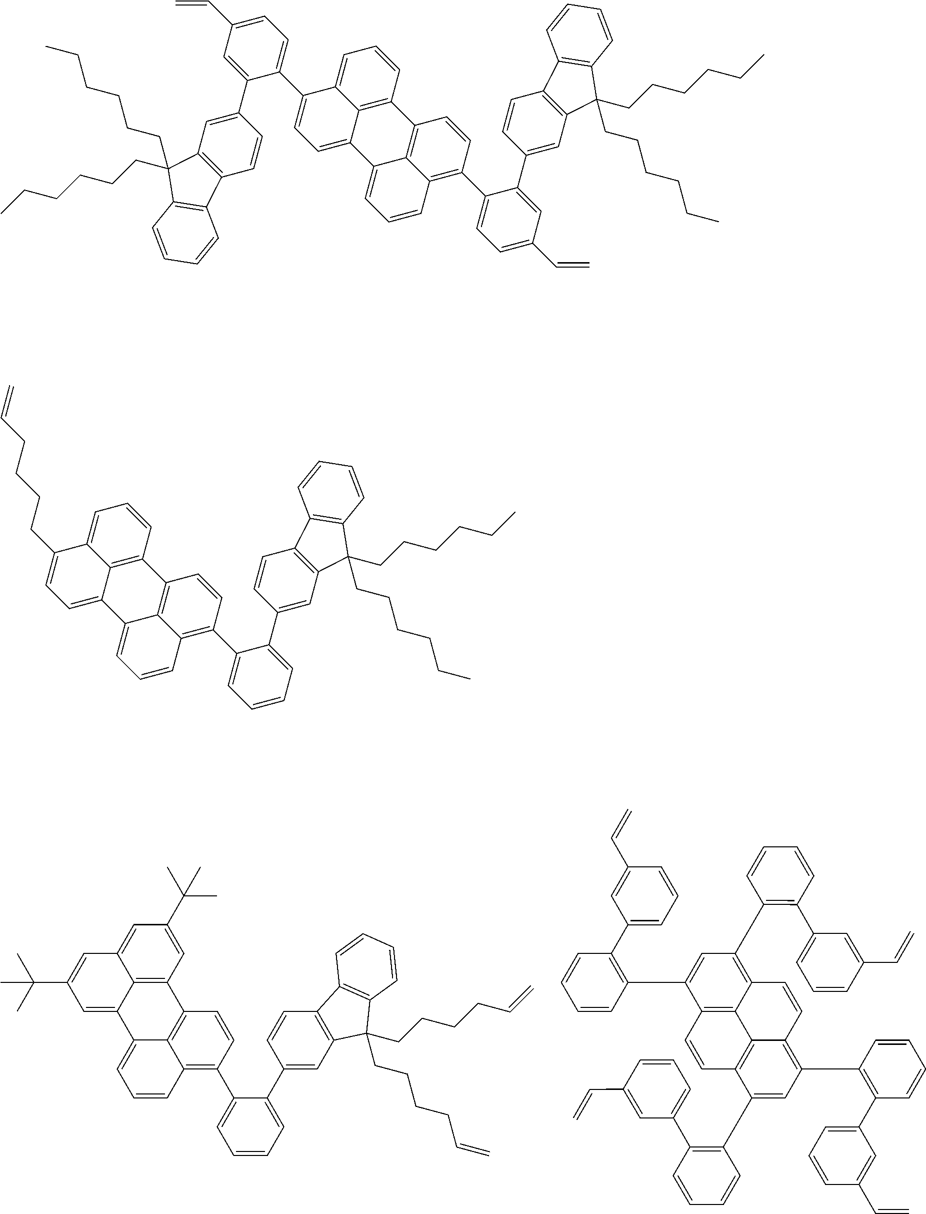

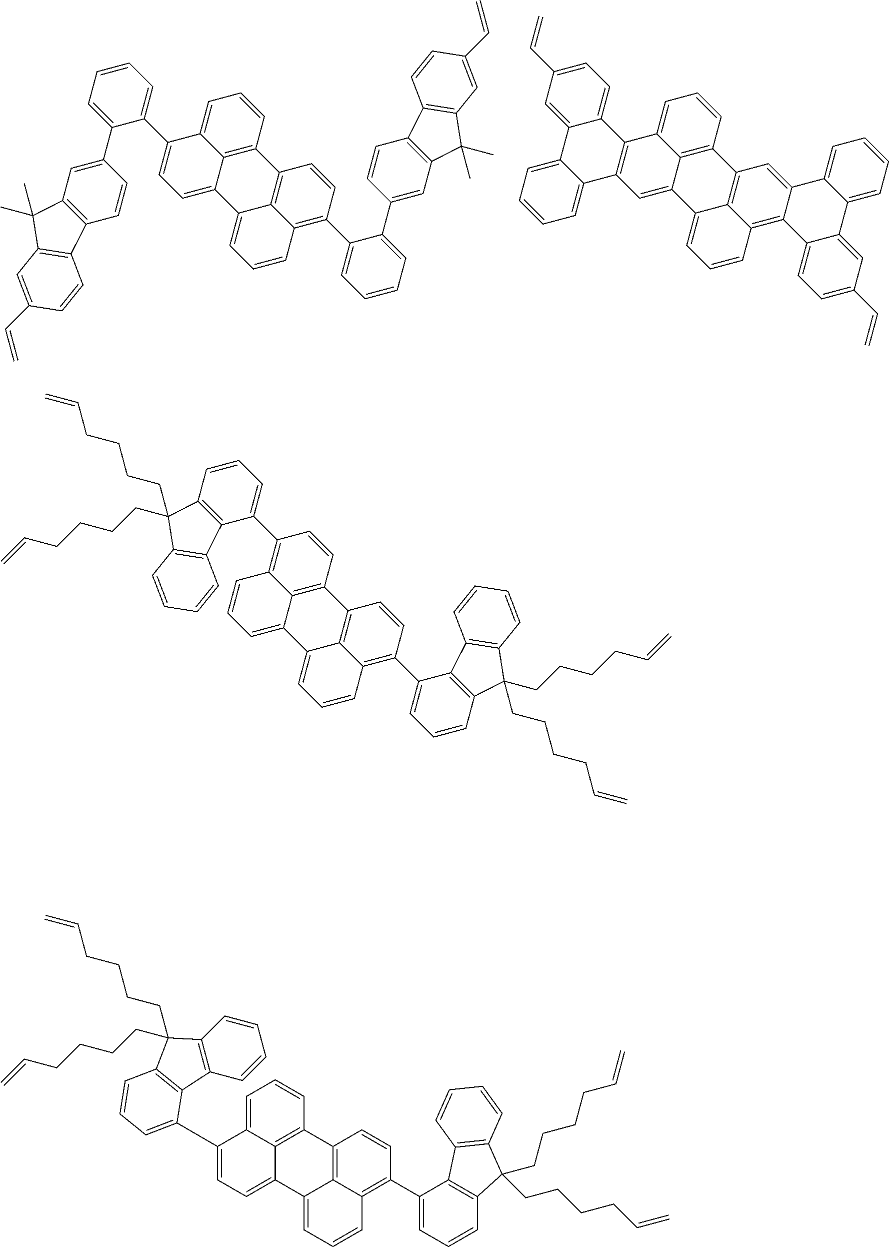

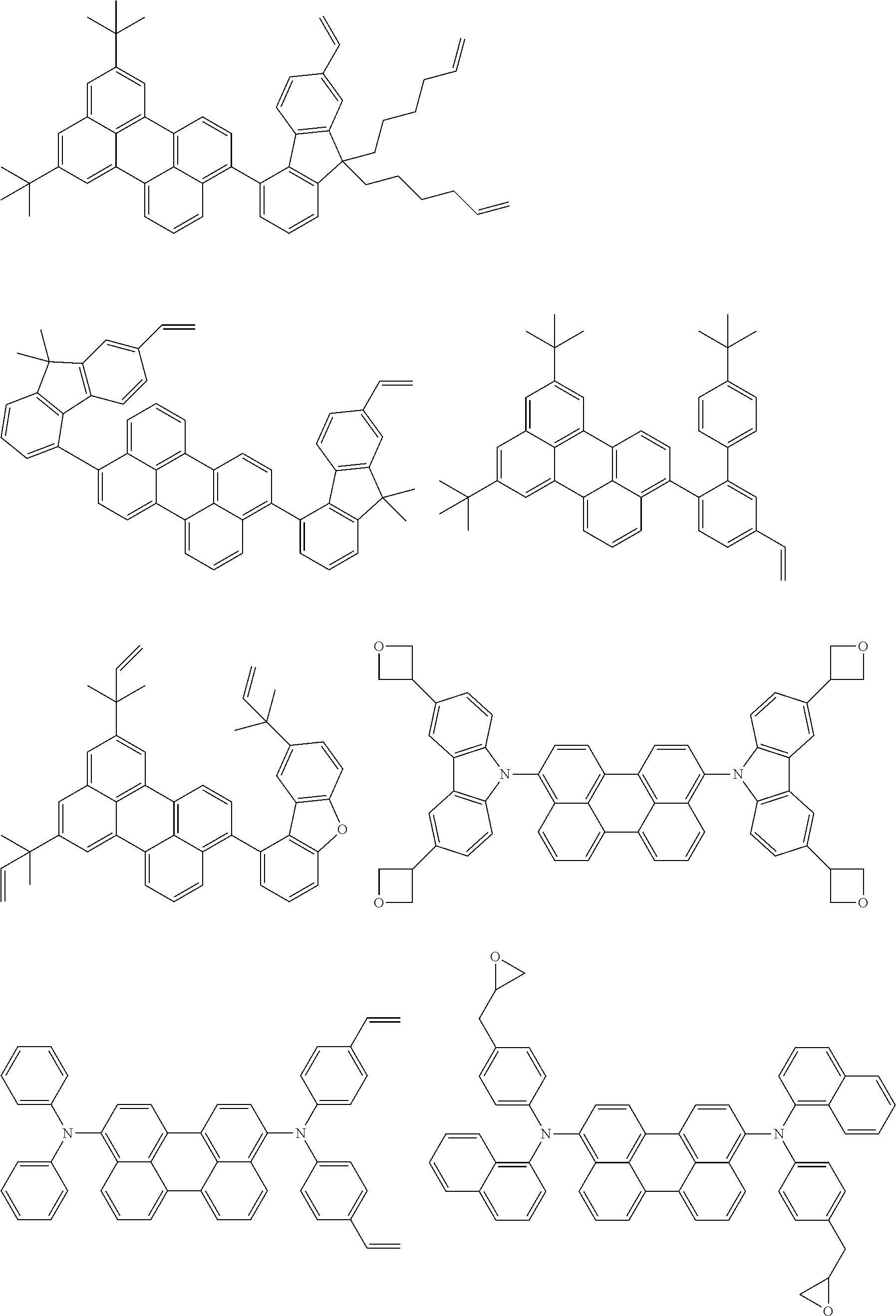

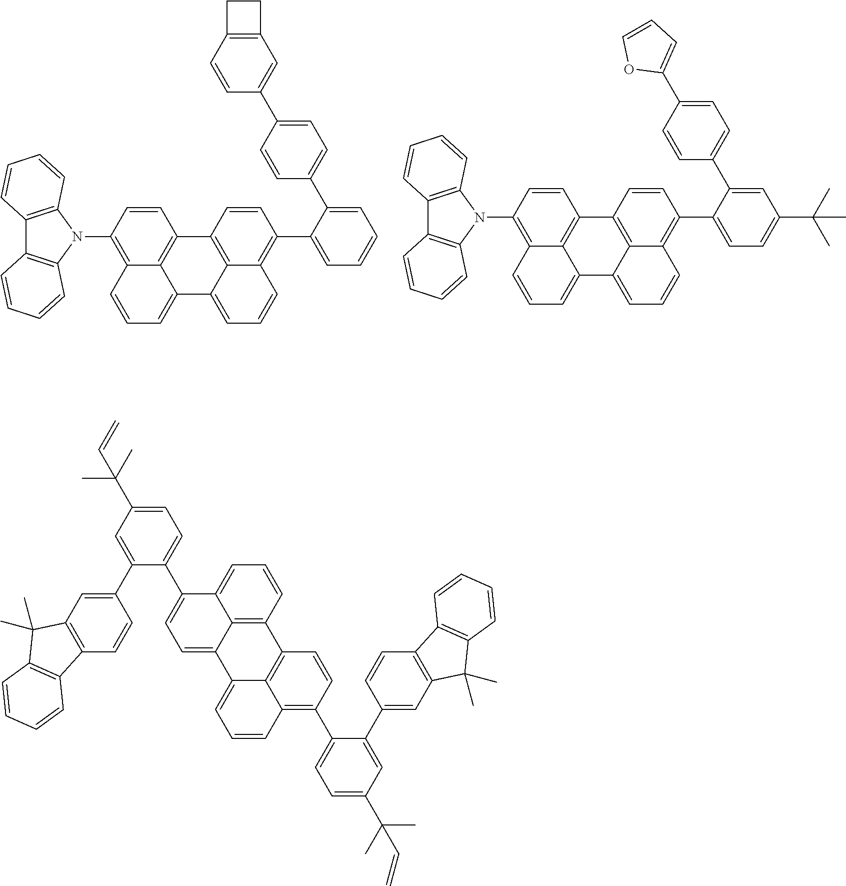

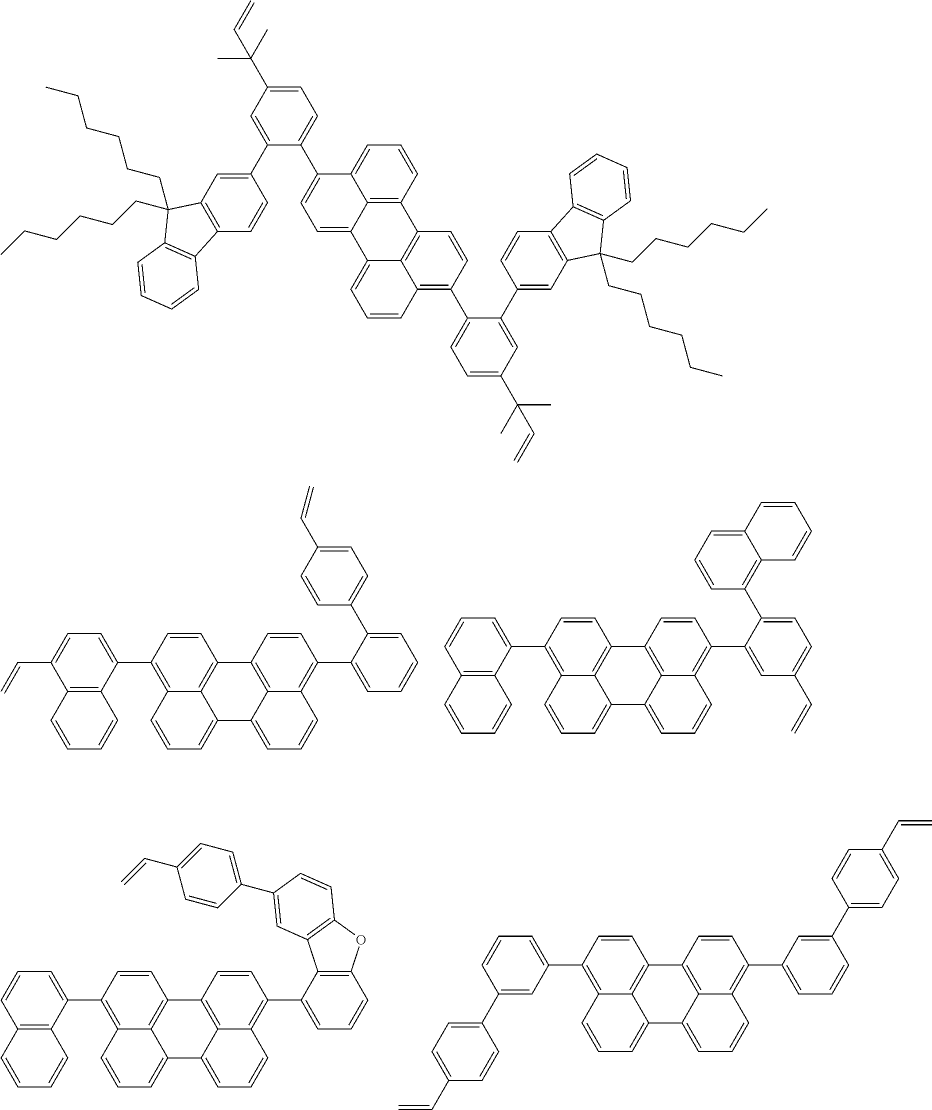

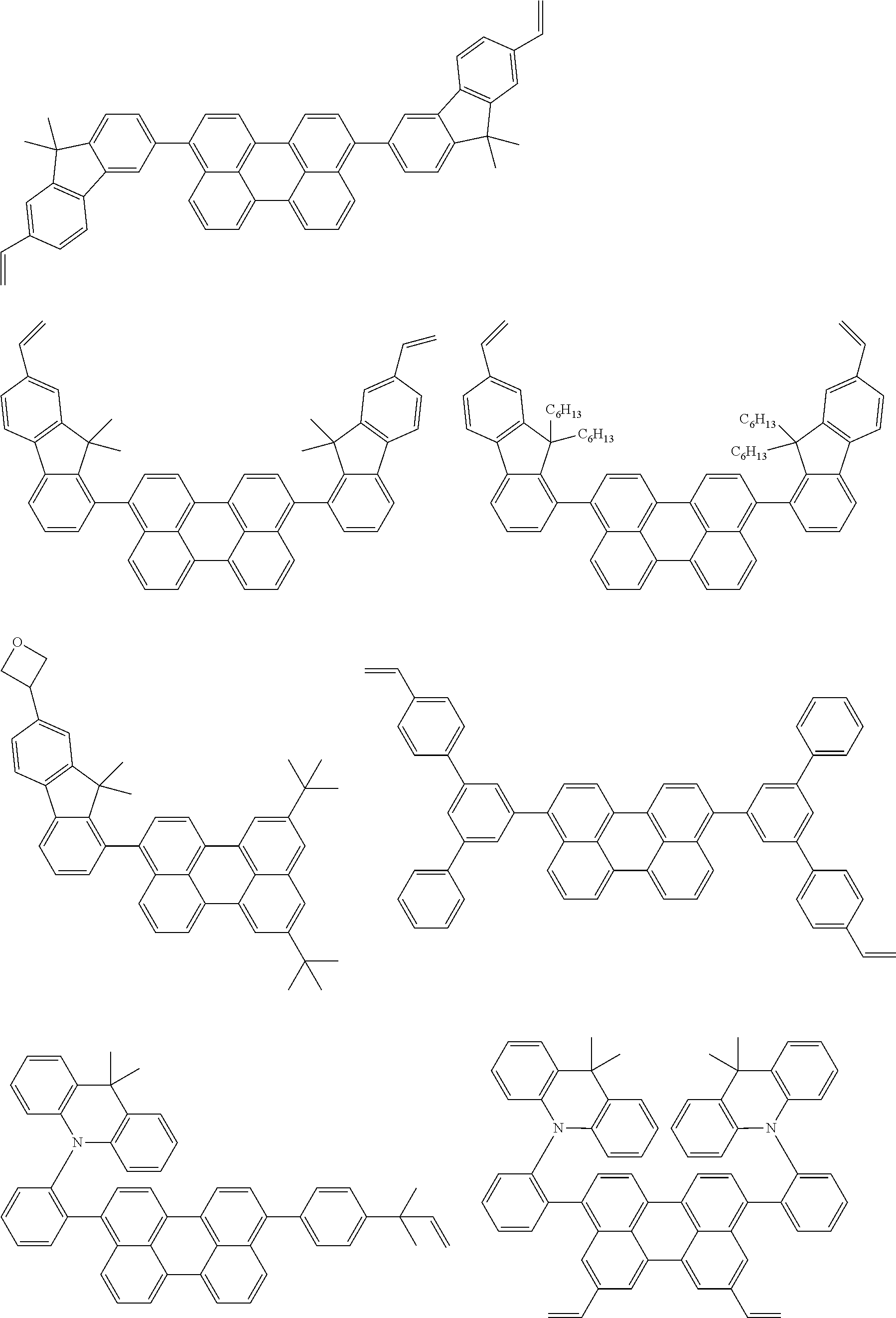

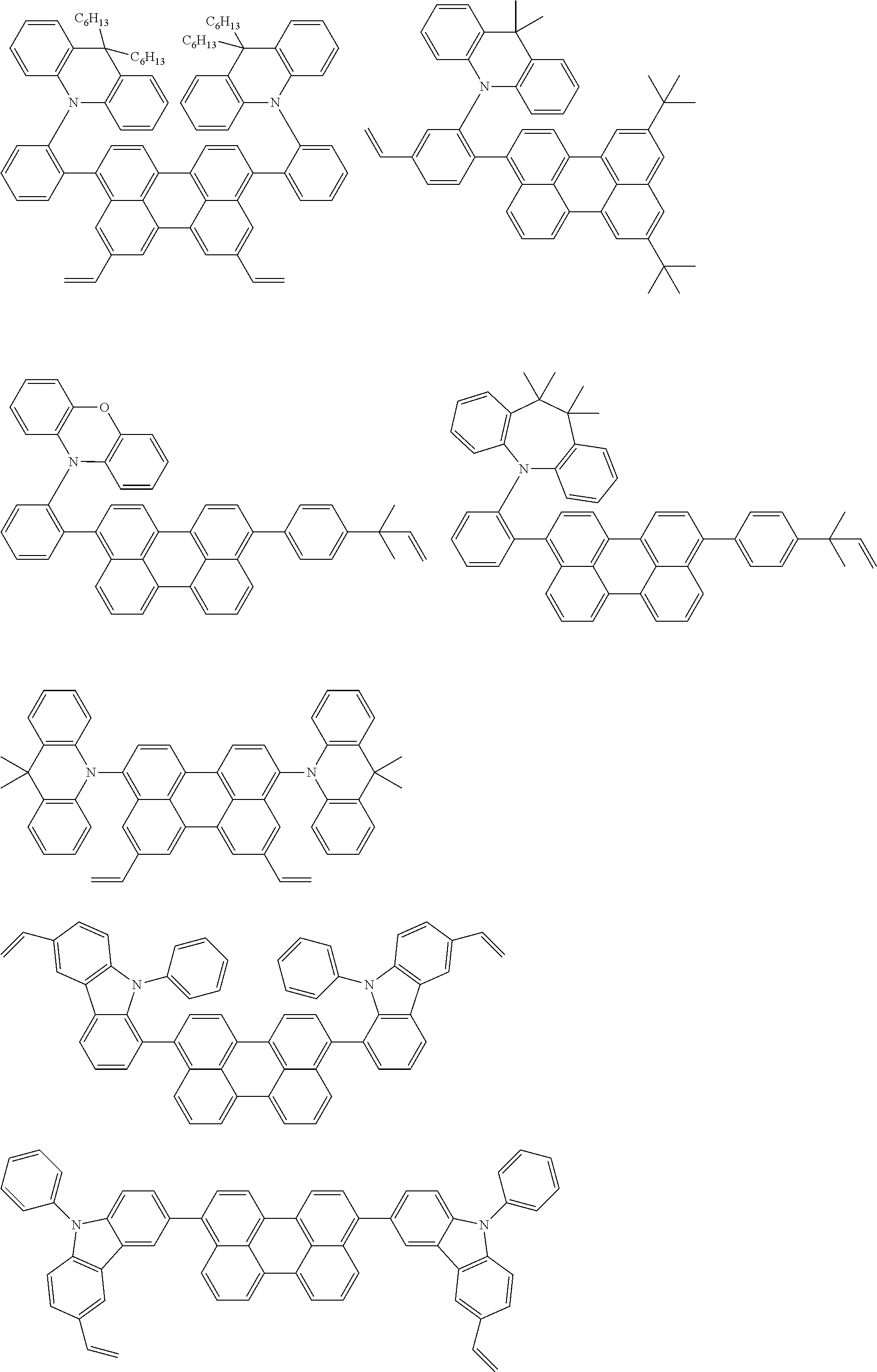

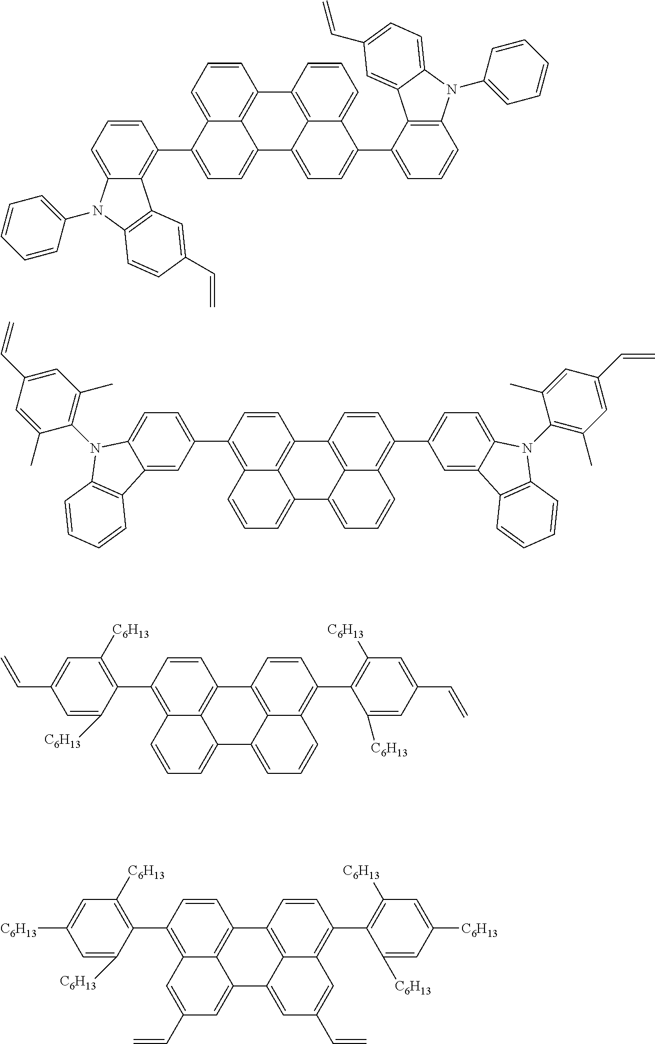

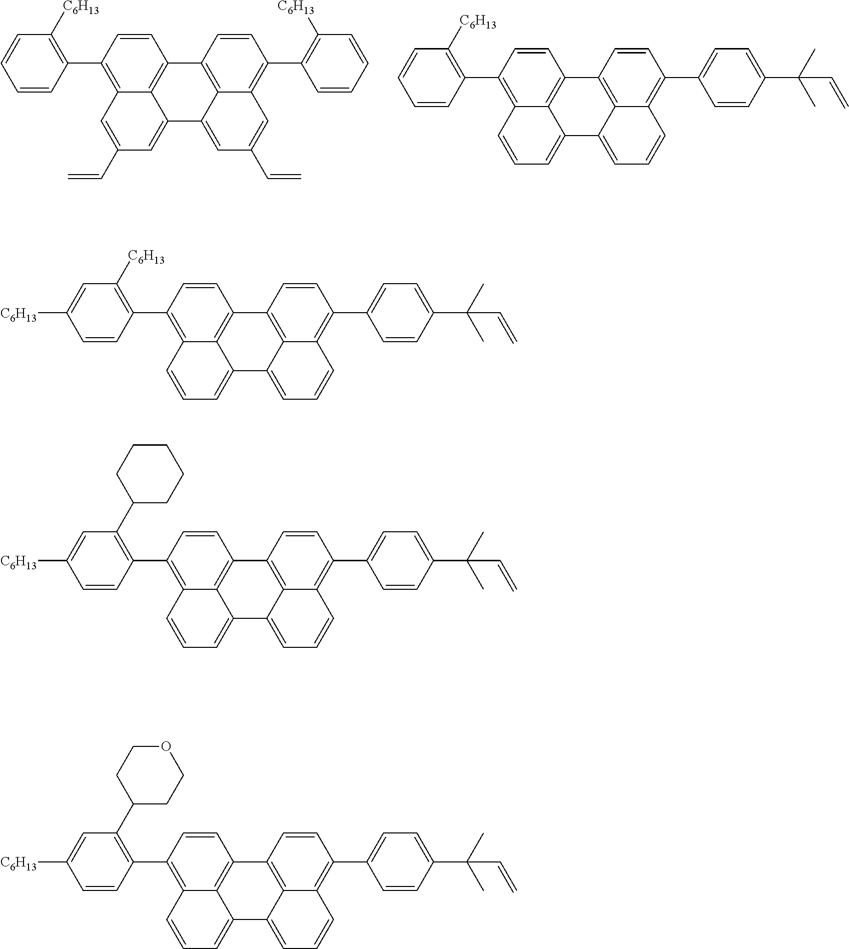

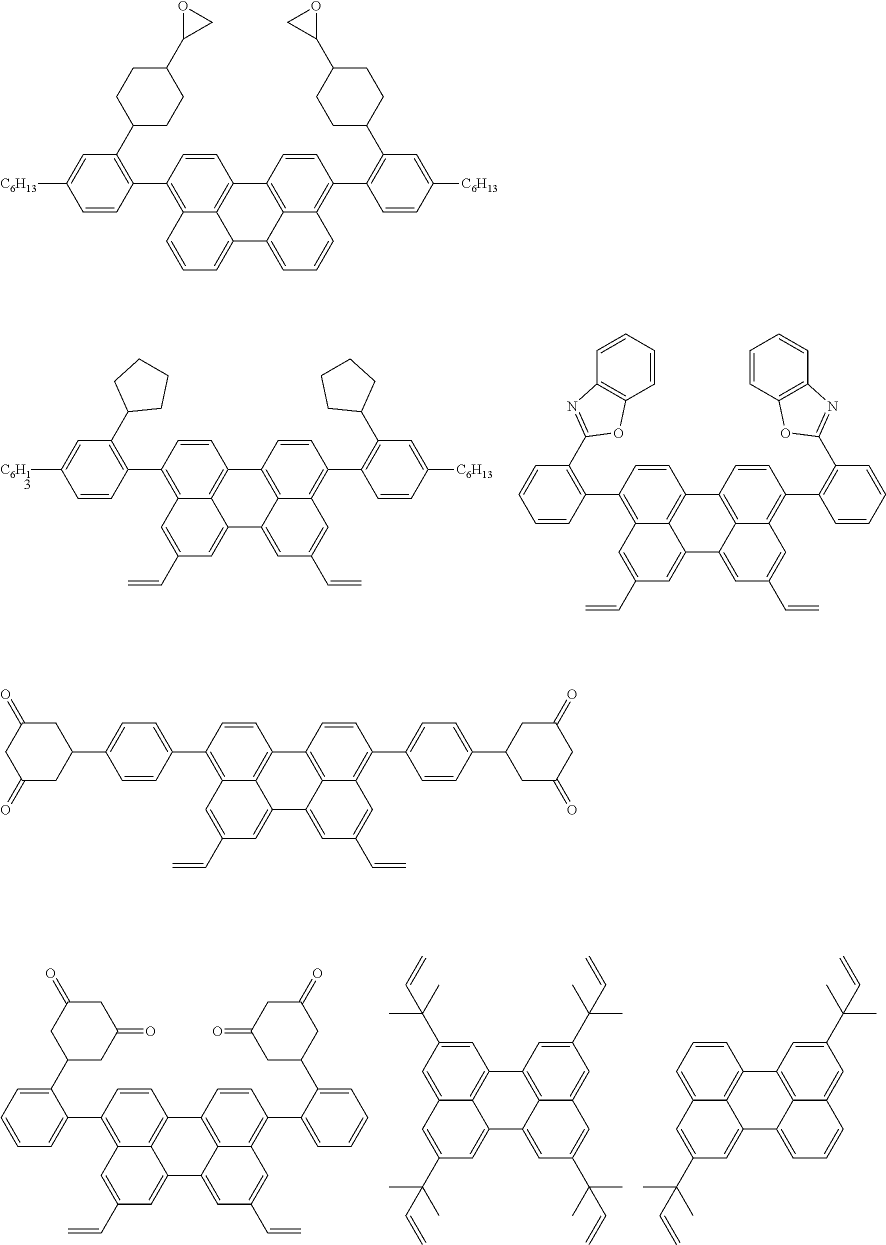

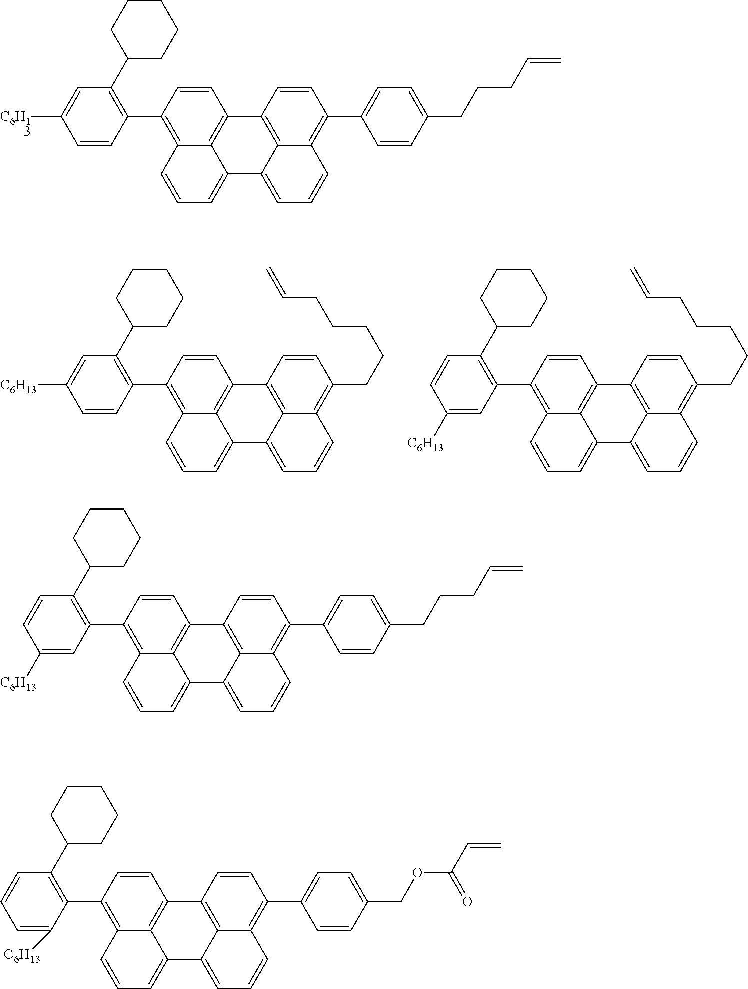

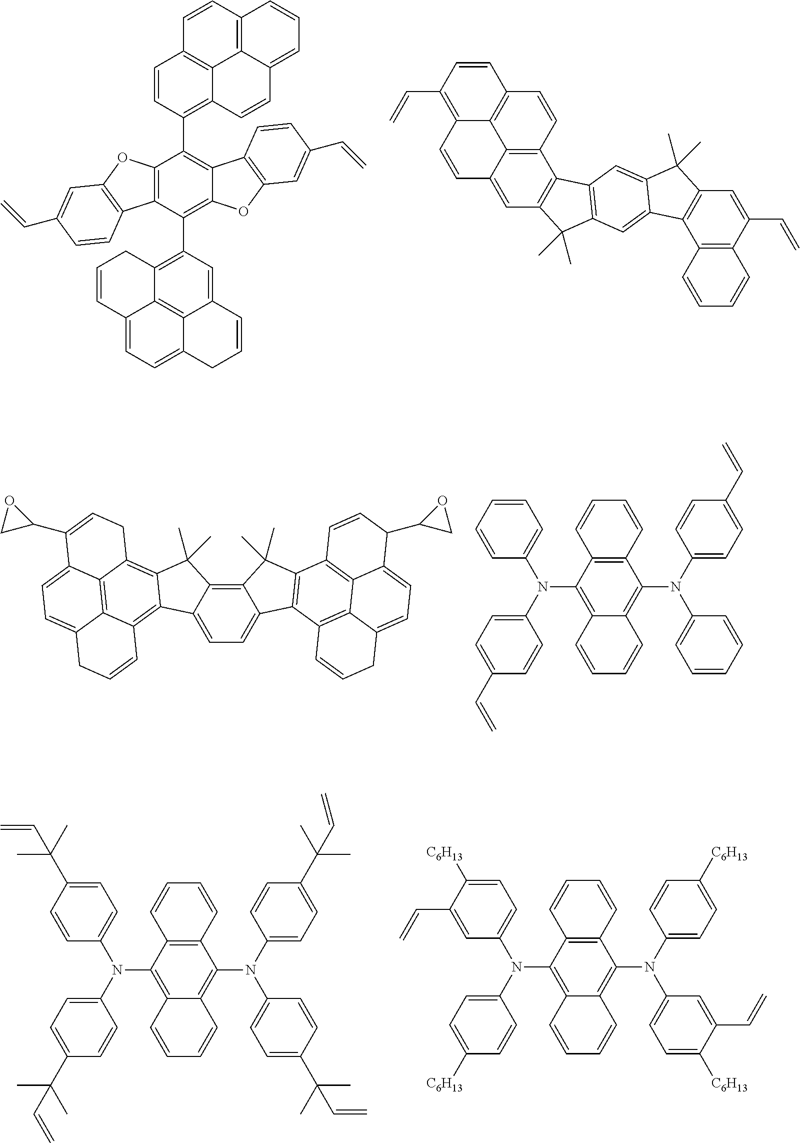

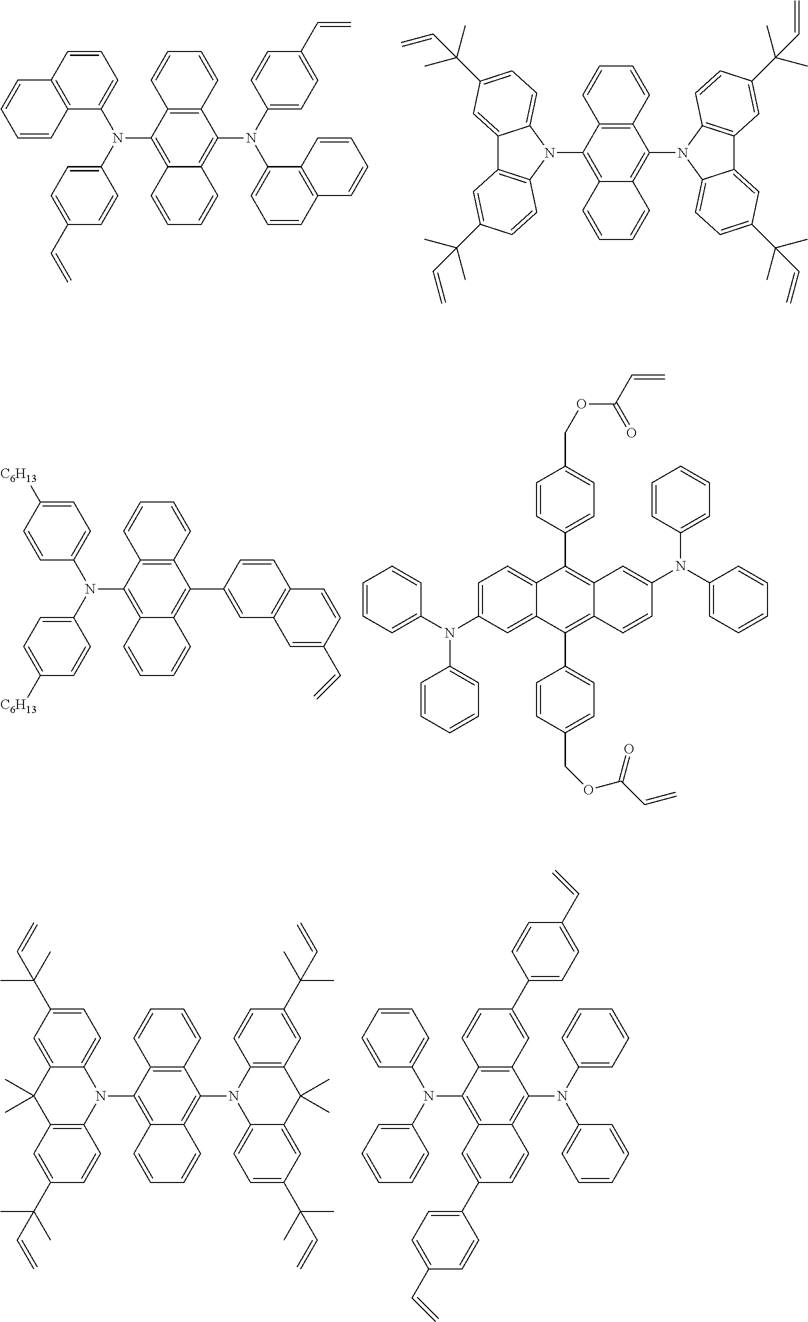

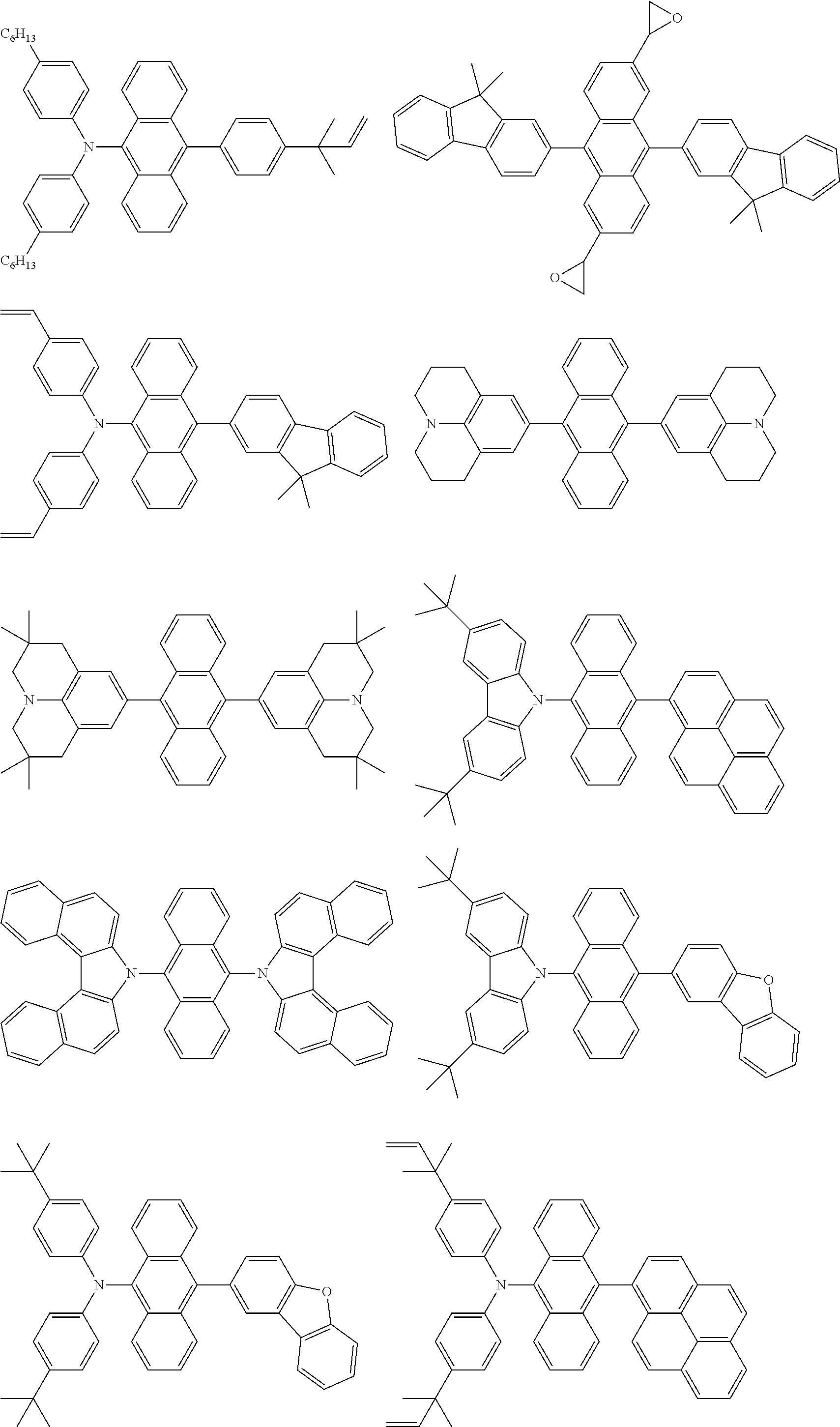

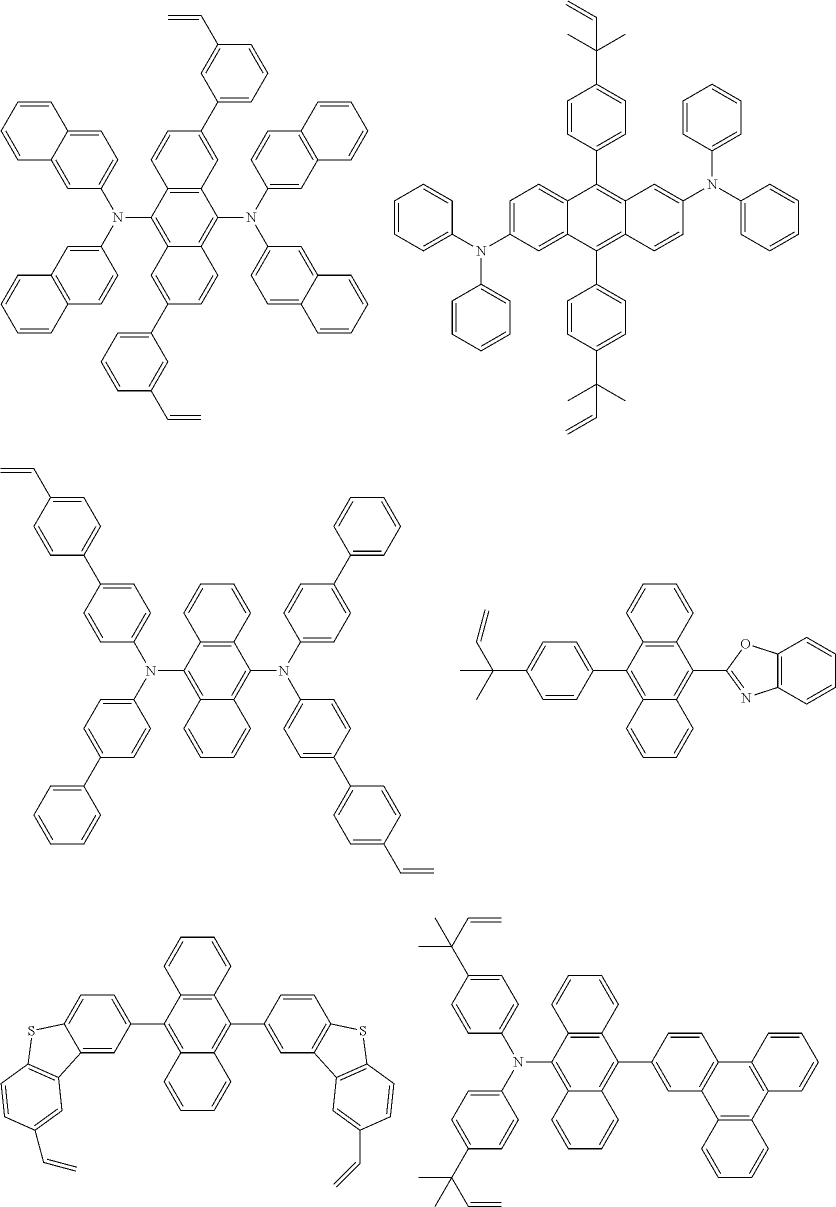

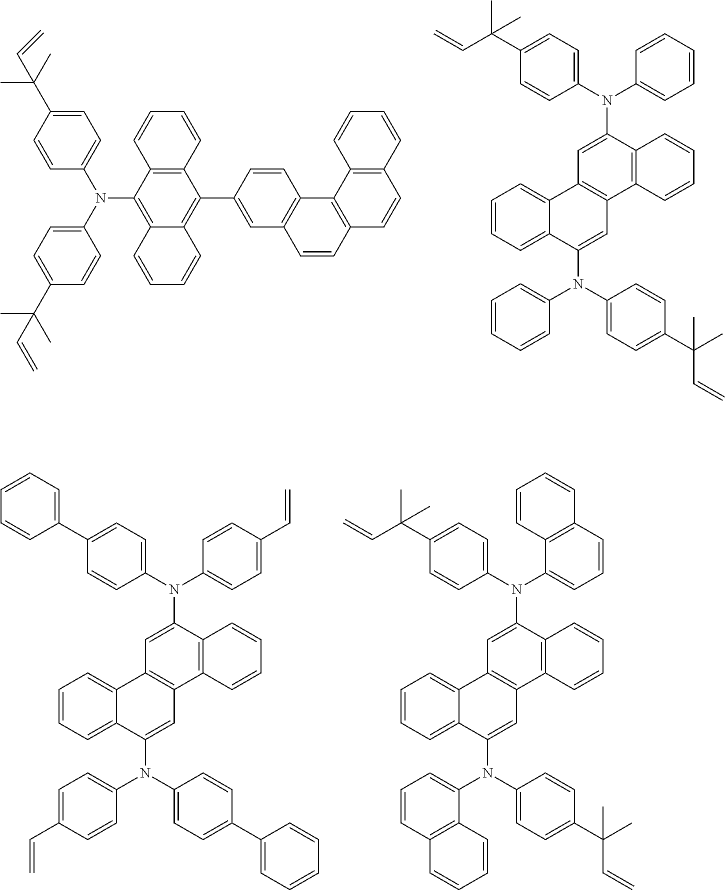 [SMILES: C=CC(C)(C)c1ccc(N(c2ccc(C(C)(C)C=C)cc2)c2c3ccccc3c(-c3ccc4c(ccc5ccc6ccccc6c54)c3)c3ccccc23)cc1 ; C=CC(C)(C)c1ccc(N(c2ccccc2)c2cc3c4ccccc4c(N(c4ccccc4)c4ccc(C(C)(C)C=C)cc4)cc3c3ccccc23)cc1 ; C=Cc1ccc(N(c2ccc(-c3ccccc3)cc2)c2cc3c4ccccc4c(N(c4ccc(C=C)cc4)c4ccc(-c5ccccc5)cc4)cc3c3ccccc23)cc1 ; C=CC(C)(C)c1ccc(N(c2cccc3ccccc23)c2cc3c4ccccc4c(N(c4ccc(C(C)(C)C=C)cc4)c4cccc5ccccc45)cc3c3ccccc23)cc1]

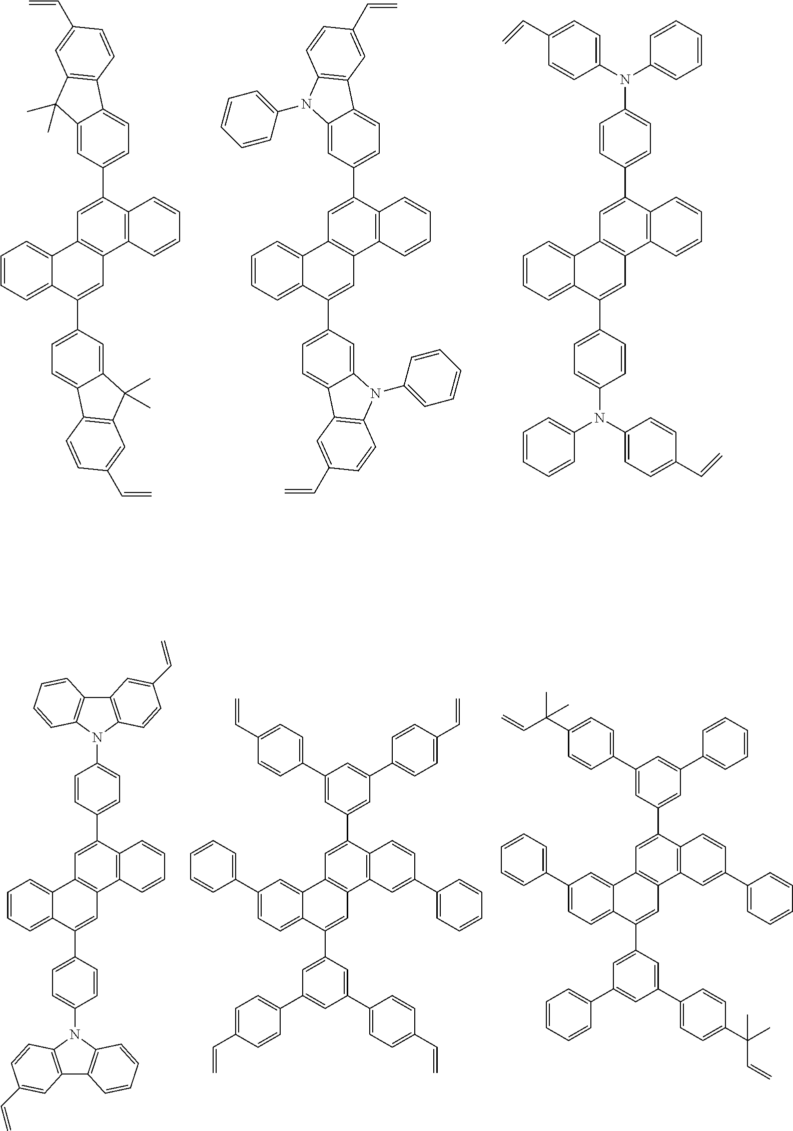

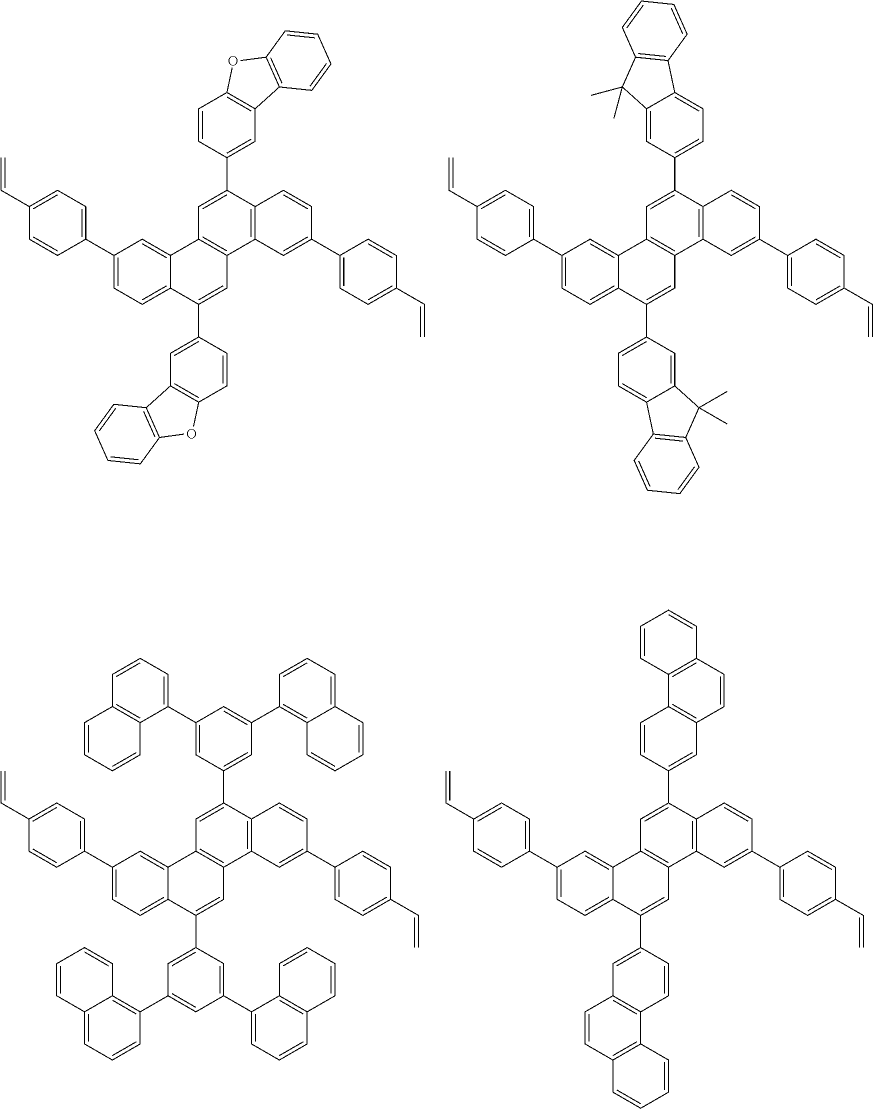

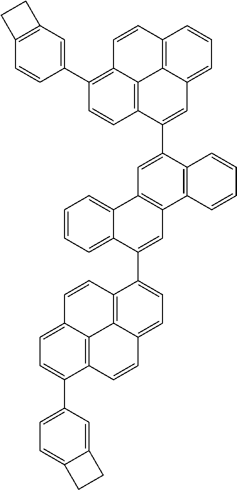 [SMILES: c1cc2ccc3c(-c4ccc5c(c4)CC5)ccc4c(-c5cc6c7ccccc7c(-c7ccc8ccc9c(-c%10ccc%11c(c%10)CC%11)ccc%10ccc7c8c%109)cc6c6ccccc56)cc(c1)c2c34]

In another aspect, the present disclosure also provides a synthetic method of the compound of formulas (1)-(4) using the reagents with active groups. These active reagents comprise at least one leaving group, such as bromine, iodine, aromatic formaldehyde, or boronic ester. The appropriate C—C coupling reactions are well-known to those skilled in the art and are described in the literature, particularly appropriate and preferred coupling reactions are the SUZUKI, STILLE, BUCHWALD-HARTWIG, HECK coupling, and WITTIG reaction.

In another aspect, the present disclosure further provides a mixture comprising at least one compound as described herein and another functional material, the another functional material is an organic functional material, which can be selected from a hole-injection material (HIM), a hole-transport material (HTM), a hole-blocking material (HBM), an electron-injection material (EIM), an electron-transport material (ETM), an electron-blocking material (EBM), an organic host material (Host), a singlet emitting material (fluorescent emitting material), a triplet emitting material (phosphorescent emitting material), a thermally activated delayed fluorescence material (TADF material), or an organic dye. These organic functional materials are described in detail, for example, in WO2010135519A1, US20090134784A1, and WO2011110277A1. The three documents are incorporated herein by reference in their entirety.

In some embodiments, the mixture comprises a compound as described herein, and an emitting material. The compound as described herein can be used as a host material, and the weight percentage of the emitting material ≤15 wt %, preferably ≤12 wt %, more preferably ≤9 wt %, further preferably ≤8 wt %, and most preferably ≤7 wt %.

In some embodiments, the emitting material is an organic fluorescent emitter.

The fluorescent emitter (singlet emitter) are described in detail below.

The singlet emitter tends to have a long conjugated π-electron system. Hitherto, there have been many examples of styryl amines and derivatives thereof as disclosed in JP2913116B and WO2001021729A1, and indenofluorenes and derivatives thereof as disclosed in WO2008006449 and WO2007140847.

In some embodiments, the singlet emitter can be selected from the group consisting of monostyrylamines, distyrylamines, tristyrylamines, tetrastyrylamines, styrenphosphines, styrenethers, and arylamines.

A monostyrylamine refers to a compound which comprises one unsubstituted or substituted styryl group and at least one amine, most preferably an aryl amine. Distyrylamine refers to a compound comprising two unsubstituted or substituted styryl groups and at least one amine, most preferably an aryl amine. Ternarystyrylamine refers to a compound which comprises three unsubstituted or substituted styryl groups and at least one amine, most preferably an aryl amine. Quaternarystyrylamine refers to a compound comprising four unsubstituted or substituted styryl groups and at least one amine, most preferably an aryl amine. Preferred styrene is stilbene, which may be further substituted. The corresponding phosphines and ethers are defined similarly as amines. Aryl amine or aromatic amine refers to a compound comprising three unsubstituted or substituted cyclic or heterocyclic aryl systems directly attached to nitrogen. At least one of these cyclic or heterocyclic aryl systems is preferably selected from fused ring systems and most preferably has at least 14 aryl ring atoms. Among the preferred examples are aryl anthramine, aryl anthradiamine, aryl pyrene amines, aryl pyrene diamines, aryl chrysene amines and aryl chrysene diamine. Aryl anthramine refers to a compound in which one diarylamino group is directly attached to anthracene, most preferably at position 9. Aryl anthradiamine refers to a compound in which two diarylamino groups are directly attached to anthracene, most preferably at positions 9,10. Aryl pyrene amines, aryl pyrene diamines, aryl chrysene amines and aryl chrysene diamine are similarly defined, where the diarylarylamino group is most preferably attached to position 1 or 1,6 of pyrene.

Examples of singlet emitters based on vinylamines and arylamines, which are also preferred, may be found in the following patent documents: WO2006000388, WO2006058737, WO2006000389, WO2007065549, WO2007115610, U.S. Pat. No. 7,250,532B2, DE102005058557A1, CN1583691A, JP08053397A, U.S. Pat. No. 6,251,531B1, US2006210830A, EP1957606A1, and US20080113101A1. The patent documents listed above are specially incorporated herein by reference in their entirety.

Examples of singlet emitters based on stilbene and its derivatives may be found in U.S. Pat. No. 5,121,029.

Further preferred singlet emitter can be selected from the group consisting of indenofluorene-amine and indenofluorene-diamine, as disclosed in WO2006122630, benzoindenofluorene-amine and benzoindenofluorene-diamine, as disclosed in WO2008006449, dibenzoindenofluorene-amine and dibenzoindenofluorene-diamine, as disclosed in WO2007140847.

Other materials that can be used as singlet emitter include polycyclic aromatic hydrocarbon compounds, in particular selected from the derivatives of the following compounds: anthracene such as 9,10-di(2-naphthyl)anthracene, naphthalene, tetraphenyl, phenanthrene, perylene such as 2,5,8,11-tetra-t-butylatedylene, indenoperylene, phenylene (benzo fused ring such as 4,4′-(bis (9-ethyl-3-carbazovinylene)-1,1′-biphenyl)), periflanthene, decacyclene, coronene, fluorene, spirobifluorene, arylpyren (e.g., US20060222886), arylenevinylene (e.g. U.S. Pat. Nos. 5,121,029, 5,130,603), cyclopentadiene such as tetraphenylcyclopentadiene, rubrene, coumarine, rhodamine, quinacridone, pyrane such as 4(dicyanoethylene)-6-(4-dimethylaminostyryl-2-methyl)-4H-pyrane (DCM), thiapyran, bis (azinyl) imine-boron compounds (e.g. US20070092753A1), bis (azinyl) methene compounds, carbostyryl compounds, oxazone, benzoxazole, benzothiazole, benzimidazole, or diketopyrrolopyrrole. Some singlet emitter materials may be found in the following patent documents: US20070252517A1, U.S. Pat. Nos. 4,769,292, 6,020,078. The patent documents listed above are specially incorporated herein by reference in their entirety.

The following are some examples of singlet emitters:

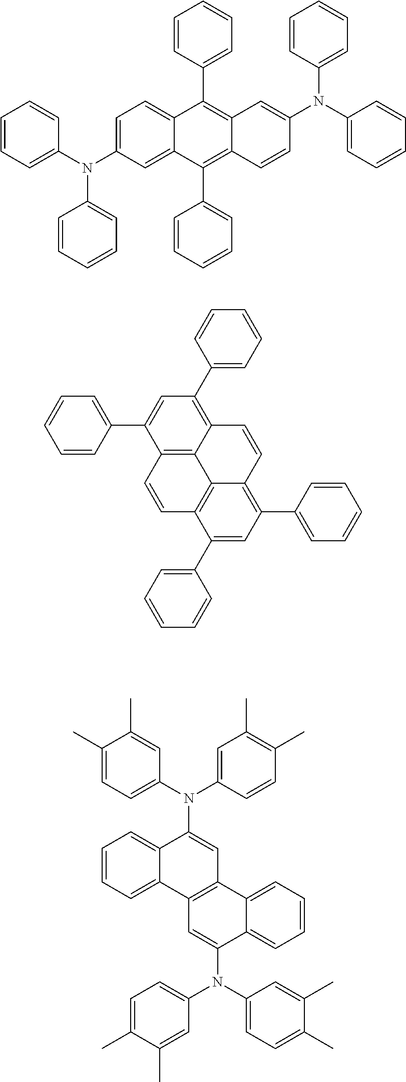

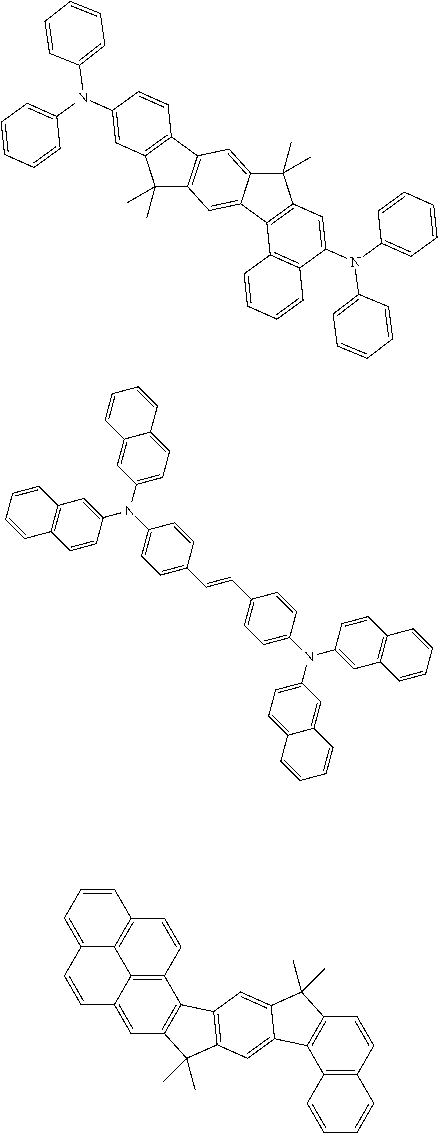

In some embodiments, the mixture comprises at least one compound (as a host material H) as described herein and an emitter E1, where 1) the emission spectrum of the compound (host material H) is on the short wavelength side of the absorption spectrum of the emitter E1, and at least partially overlaps with the absorption spectrum of the emitter E1; 2) the FWHM of the emission spectrum of the emitter E1≤55 nm.

In yet another aspect, the present disclosure further provides a formulation comprising at least one compound as described herein, at least one organic solvent, and/or an organic resin.

In some embodiments, the formulation further comprises an emitter E1, 1) the emission spectrum of the compound is on the short wavelength side of the absorption spectrum of the emitter E1, and at least partially overlaps with the absorption spectrum of the emitter E1; 2) the FWHM of the emission spectrum of the emitter E1 nm.

In some embodiments, the formulation comprises an organic resin; in some embodiments, the formulation comprises two or more organic resins; in some embodiments, the formulation comprises three or more organic resins.

For the purposes of the present disclosure, the organic resin refers to a resin prepolymer or a resin formed after the resin prepolymer is crosslinked or cured.

The organic resins suitable for the present disclosure include, but not limited to: polystyrene, polyacrylate, polymethacrylate, polycarbonate, polyurethane, polyvinylpyrrolidone, polyvinyl acetate, polyvinyl chloride, polybutylene, polyethylene glycol, polysiloxane, polyacrylate, epoxy resin, polyvinyl alcohol, polyacrylonitrile, polyvinylidene chloride (PVDC), polystyrene-acrylonitrile (SAN), polybutylene terephthalate (PBT), polyethylene terephthalate (PET), polyvinyl butyrate (PVB), polyvinyl chloride (PVC), polyamide, polyoxymethylene, polyimide, polyetherimide, and mixtures thereof.

Further, the organic resins suitable for the present disclosure include, but not limited to, those prepared by the homopolymerization or copolymerization of the following monomers (resin prepolymers): styrene derivatives, acrylate derivatives, acrylonitrile derivatives, acrylamide derivatives, vinyl ester derivatives, vinyl ether derivatives, maleimide derivatives, conjugated diene derivatives.

Examples of styrene derivatives include, but not limited to alkylstyrenes, such as α-methylstyrene, o-, m-, p-methylstyrene, p-butylstyrene; especially 4-tert-butylstyrene; alkoxystyrene, such as p-methoxystyrene, p-butoxystyrene, p-tert-butoxystyrene.

Examples of acrylate derivatives include, but not limited to methyl acrylate, methyl methacrylate, ethyl acrylate, ethyl methacrylate, n-propyl acrylate, n-propyl methacrylate, isopropyl acrylate, isopropyl methacrylate, n-butyl acrylate, n-butyl methacrylate, isobutyl acrylate, isobutyl methacrylate, sec-butyl acrylate, sec-butyl methacrylate, tert-butyl acrylate, tert-butyl methacrylate, 2-hydroxyethyl acrylate, 2-hydroxyethyl methacrylate, 2-hydroxypropyl acrylate, 2-hydroxypropyl methacrylate, 3-hydroxypropyl acrylate, 3-hydroxypropyl methacrylate, 2-hydroxybutyl acrylate, 2-hydroxybutyl methacrylate, 3-hydroxybutyl acrylate, 3-hydroxybutyl methacrylate, 4-hydroxybutyl acrylate, 4-hydroxybutyl methacrylate, allyl acrylate, allyl methacrylate, benzyl acrylate, benzyl methacrylate, cyclohexyl acrylate, cyclohexyl methacrylate, phenyl acrylate, phenyl methacrylate, 2-methoxyethyl acrylate, 2-methoxyethyl methacrylate, 2-phenoxyethyl acrylate, 2-phenoxyethyl methacrylate, methoxydiethylene glycol acrylate, methoxydiethylene glycol methacrylate, methoxytriethylene glycol acrylate, methoxytriethylene glycol methacrylate, methoxypropylene glycol acrylate, methoxypropylene glycol methacrylate, methoxy dipropylene glycol acrylate, methoxydipropylene glycol methacrylate, isobornyl acrylate, isobornyl methacrylate, dicyclopentadiene acrylate, dicyclopentadiene methacrylate, adamantane (meth) acrylate, norbornene (meth) acrylate, 2-hydroxy-3-phenoxypropyl acrylate, 2-hydroxy-3-phenoxypropyl methacrylate, glyceryl monoacrylate, and glyceryl monostearate; 2-aminoethyl acrylate, 2-aminoethyl methacrylate, 2-dimethylaminoethyl acrylate, 2-dimethylaminoethyl methacrylate, N,N-dimethylaminoethyl (meth) acrylic acid, N,N-diethylaminoethyl (meth) acrylate, 2-dimethylaminopropyl methacrylate, 3-aminopropyl acrylate, 3-aminopropyl methacrylate, N,N-dimethyl-1,3-propane diamine (meth) acrylate, 3-dimethylaminopropyl acrylate, 3-dimethylaminopropyl methacrylate, glycidyl acrylate, and glycidyl methacrylate.

Examples of acrylonitrile derivatives include, but not limited to acrylonitrile, methacrylonitrile, α-chloroacrylonitrile, and vinylidene cyanide.

Examples of acrylamide derivatives include, but not limited to acrylamide, methacrylamide, a-chloroacrylamide, N-2-hydroxyethyl acrylamide, and N-2-hydroxyethyl methacrylamide.

Examples of vinyl ester derivatives include, but not limited to vinyl acetate, vinyl propionate, vinyl butyrate, and vinyl benzoate.

Examples of vinyl ether derivatives include, but not limited to vinyl methyl ether, vinyl ethyl ether, and allyl glycidyl ether.

Examples of maleimide derivatives include, but not limited to maleimide, benzylmaleimide, N-phenylmaleimide, and N-cyclohexylmaleimide.

Examples of conjugated diene derivatives include, but not limited to 1,3-butadiene, isoprene, and chloroprene.

The homopolymers or copolymers can be prepared by free radical polymerization, cationic polymerization, anionic polymerization, or organometallic catalytic polymerization (for example Ziegler-Natta catalysis). The process of polymerization can be suspension polymerization, emulsion polymerization, solution polymerization, or bulk polymerization.

The number average molecular weight Mn (as determined by GPC) of the organic resins is generally in the range of 10 000 g/mol to 1 000 000 g/mol, preferably in the range of 20 000 g/mol to 750 000 g/mol, more preferably in the range of 30 000 g/mol to 500 000 g/mol.

In some embodiments, the organic resin is a thermosetting resin or an UV curable resin. In some embodiments, the organic resin is cured by a method that will enable roll-to-roll processing.

Thermosetting resins require curing in which they undergo an irreversible process of molecular cross-linking, which makes the resin non-fusible. In some embodiments, the thermosetting resin is an epoxy resin, a phenolic resin, a vinyl resin, a melamine resin, a urea-formaldehyde resin, an unsaturated polyester resin, a polyurethane resin, an allyl resin, an acrylic resin, a polyamide resin, a polyamide-imide resin, a phenol-amide polycondensation resin, an urea-melamine polycondensation resin, or combinations thereof.

In some embodiments, the thermosetting resin is an epoxy resin. The epoxy resins are easy to cure and do not give off volatiles or generate by-products from a wide range of chemicals. The epoxy resins can also be compatible with most substrates and tend to readily wet surfaces. See also Boyle, M. A. et al., “Epoxy Resins”, Composites, Vol. 21, ASM Handbook, pages 78-89 (2001).

In some embodiments, the organic resin is a silicone thermosetting resin. In some embodiments, the silicone thermosetting resin is OE6630A or OE6630B (Dow Corning Corporation (Auburn, Michigan.)).

In some embodiments, the formulation comprises an organic solvent. In some embodiments, the formulation comprises two or more organic solvents. In some embodiments, the formulation comprises three or more organic solvents.

In some embodiments, the formulation as described herein is a solution.

In some embodiments, the formulation as described herein is a dispersion.

The formulation in the embodiments of the present disclosure may comprise the compound as described herein of 0.01 wt % to 20 wt %, preferably 0.1 wt % to 20 wt %, more preferably 0.2 wt % to 20 wt %, and most preferably 1 wt % to 15 wt %.

Using the formulation as described herein, the color conversion layer may be fabricated by ink-jet printing, transfer printing, photolithography, etc. In this case, the color conversion material needs to be dissolved alone or together with other materials in an organic solvent, to form an ink. The mass concentration of the color conversion material in the ink is not less than 0.1 wt %. The color conversion ability of the color conversion layer can be tuned by adjusting the concentration of the color conversion material in the ink and the thickness of the color conversion layer. In general, the higher the concentration of the color conversion material or the thickness of the layer, the higher the color conversion efficiency of the color conversion layer would be.

Other materials that can be added into the ink include, but not limited to the following materials: polyethylene, polypropylene, polystyrene, polycarbonate, polyacrylate, polyvinyl pyrrolidone, polyvinyl alcohol, polyvinyl acetate, polyethylene glycol, polysiloxane, polyacrylonitrile, polyvinyl chloride, polyvinylidene chloride, polyethylene terephthalate, polybutylene terephthalate, polyvinyl butyrate, polyamide, polyoxymethylene, polyimide, polyether-ether-ketone, polysulfone, polyarylether, polyaramide, cellulose, modified cellulose, acetate fiber, nitrocellulose, and mixtures thereof.

In some embodiments, the at least one of organic solvent is selected from alcohols, esters, aromatic ketones, aromatic ethers, aliphatic ketones, aliphatic ethers, borates, phosphorates, or mixtures of two or more of them.

In some embodiments, the suitable and preferred organic solvents include aliphatics, alicyclics, aromatics, amines, thiols, amides, nitriles, esters, ethers, polyethers, alcohols, diols, or polyols.

In some embodiments, the alcohol represents an organic solvent of the suitable class. Preferred alcohols include alkylcyclohexanols, particularly methylated aliphatic alcohols, naphthols, etc.

Other examples of the suitable alcohol solvents include dodecanol, phenyltridecanol, benzyl alcohol, ethylene glycol, ethylene glycol methyl ether, glycerol, propylene glycol, propylene glycol, 1-ethoxy-2-propanol, etc.

The organic solvent may be used alone or as mixtures of two or more organic solvents. In some embodiments, the formulation comprises a compound as described herein and at least one organic solvent, and further comprising another organic solvent. Examples of the another organic solvent include, but not limited to: methanol, ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 1,4-dioxane, acetone, methyl ethyl ketone, 1,2-dichloroethane, 3-phenoxytoluene, 1,1,1-trichloroethane, 1,1,2,2-tetrachloroethane, ethyl acetate, butyl acetate, dimethylformamide, dimethylacetamide, dimethylsulfoxide, tetrahydronaphthalene, decalin, indene, and/or any mixture thereof.

In some embodiments, in the formulation as described herein, the another organic solvent is selected from aromatic, heteroaromatic, esters, aromatic ketones, aromatic ethers, aliphatic ketones, aliphatic ethers, alicyclic or olefin compounds, borates, phosphorates, or mixtures of two or more of them.

Examples of aromatic or heteroaromatic solvents as described herein include, but not limited to: 1-tetralone, 3-phenoxytoluene, acetophenone, 1-methoxynaphthalene, p-diisopropylbenzene, amylbenzene, tetrahydronaphthalene, cyclohexylbenzene, chloronaphthalene, 1,4-dimethylnaphthalene, 3-isopropylbiphenyl, p-methylcumene, dipentylbenzene, o-diethylbenzene, m-diethylbenzene, p-diethylbenzene, 1,2,3,4-tetramethyl benzene, 1,2,3,5-tetramethyl benzene, 1,2,4,5-tetramethyl benzene, butylbenzene, dodecyl benzene, 1-methylnaphthalene, 1,2,4-trichlorobenzene, 1,3-dipropoxybenzene, 4,4-difluorodiphenylmethane, diphenyl ether, 1,2-dimethoxy-4-(1-propenyl) benzene, diphenylmethane, 2-phenylpyridine, 3-phenylpyridine, 2-phenoxymethyl ether, 2-phenoxytetrahydrofuran, ethyl-2-naphthyl ether, N-methyldiphenylamine, 4-isopropylbiphenyl, α, α-dichlorodiphenylmethane, 4-(3-phenylpropyl) pyridine, benzyl benzoate, 1,1-bis (3,4-dimethylphenyl) ethane, 2-isopropylnaphthalene, dibenzyl ether, etc.

In some embodiments, the another suitable and preferred organic solvents are aliphatics, alicyclics, aromatics, amines, thiols, amides, nitriles, esters, ethers, or polyethers.

The another organic solvent may be a cycloalkane, such as decahydronaphthalene.

In some embodiments, the formulation as used herein comprises at least 50 wt % of an alcoholic solvent, preferably at least 80 wt %, particularly preferably at least 90 wt %.

In some embodiments, the organic solvent particularly suitable for the present disclosure is a solvent having Hansen solubility parameters in the following ranges:
δ_{d} (dispersion force) is in the range of 17.0 MPa^{1/2} to 23.2 MPa^{1/2}, especially in the range of 18.5 MPa^{1/2} to 21.0 MPa^{1/2};
δₚ (polarity force) is in the range of 0.2 MPa^{1/2} to 12.5 MPa^{1/2}, especially in the range of 2.0 MPa^{1/2} to 6.0 MPa^{1/2};
δₕ (hydrogen bonding force) is in the range of 0.9 MPa^{1/2} to 14.2 MPa^{1/2}, especially in the range of 2.0 MPa^{1/2} to 6.0 MPa^{1/2}.

In the formulation as described herein, the boiling point parameter should be taken into account when selecting the organic solvents. In the present disclosure, the boiling points of the organic solvents ≥150° C.; preferably ≥180° C.; more preferably ≥200° C.; further preferably ≥250° C.; and most preferably ≥275° C. or ≥300° C. The boiling points in these ranges are beneficial in terms for preventing nozzle clogging of the inkjet printhead. The organic solvent can be evaporated from solution system to form a functional film.

In some embodiments, in the formulation as used herein,
1) the viscosity is in the range of 1 cps to 100 cps at 25° C.; and/or
2) the surface tension is in the range of 19 dyne/cm to 50 dyne/cm at 25° C.

In the formulation as used herein, the surface tension parameter should be taken into account when selecting the organic solvents. The suitable surface tension parameters of the inks are suitable for the particular substrate and particular printing method. For example, for the ink-jet printing, in some embodiments, the surface tension of the organic solvent at 25° C. is in the range of 19 dyne/cm to 50 dyne/cm, more preferably in the range of 22 dyne/cm to 35 dyne/cm, and most preferably in the range of 25 dyne/cm to 33 dyne/cm.

In some embodiments, the surface tension of the ink as used herein at 25° C. is in the range of 19 dyne/cm to 50 dyne/cm; more preferably in the range of 22 dyne/cm to 35 dyne/cm; and most preferably in the range of 25 dyne/cm to 33 dyne/cm.

In the formulation as used herein, the viscosity parameters of the ink should be taken into account when selecting the organic solvents. The viscosity can be adjusted by election different methods, such as by the suitable organic solvent and the concentration of functional materials in the ink. In some embodiments, the viscosity of the organic solvent is less than 100 cps, preferably less than 50 cps, and most preferably from 1.5 cps to 20 cps. The viscosity herein refers to the viscosity during printing at the ambient temperature that is generally at 15-30° C., preferably at 18-28° C., more preferably at 20-25° C., and most preferably at 23-25° C. The resulting formulation will be particularly suitable for ink-jet printing.

In some embodiments, the viscosity of the formulation as used herein at 25° C. is in the range of about 1 cps to 100 cps; preferably in the range of 1 cps to 50 cps; and most preferably in the range of 1.5 cps to 20 cps.

The ink obtained from the organic solvent satisfying the above-mentioned boiling point parameter, surface tension parameter and viscosity parameter can form a functional film with uniform thickness and formulation property.

Salts are difficult to be purified, and the remaining impurities would influence the optoelectronic performance of the devices. For the purposes of the present disclosure, in some embodiments, the formulation or the mixture as used herein does not comprise any salts, and preferably does not comprise any organic acid salts formed by organic acids and metals. In terms of cost, the present disclosure preferably excludes organic acid salts comprising transition metals or lanthanide elements.

In yet another aspect, the present disclosure further provides an organic functional film comprising a compound, or a mixture as described herein, or prepared using a formulation as described herein. Preferably, the organic functional film is made from a formulation as described herein.

In yet another aspect, the present disclosure further provides a method for preparing the organic functional film, as shown in the following steps:
1) Prepare a formulation as used herein.
2) The formulation is coated on a substrate by printing or coating to form a film, where the method of printing or coating is selected from the group consisting of ink-jet printing, nozzle printing, typographic printing, screen printing, dip coating, spin coating, blade coating, roller printing, torsional roll printing, planographic printing, flexographic printing, rotary printing, spray coating, brush or pad printing, and slit die coating.
3) The obtained film is heated at 50° C. and above, optionally in combination with ultraviolet irradiation, to allow the film to undergo a crosslinking reaction and be cured.

The thickness of the organic functional film is generally from 50 nm to 200 μm, preferably from 100 nm to 150 μm, more preferably from 500 nm to 100 μm, further preferably from 1 μm to 50 μm, and most preferably from 1 μm to 20 μm.

In some embodiments, the thickness of the organic functional film is between 20 nm and 20 μm, preferably <15 μm, more preferably <10 μm, even more preferably <8 μm, particularly preferably <6 μm, further preferably <4 μm, and most preferably <2 μm.

A further purpose of the present disclosure is to provide the use of the compound and mixture thereof in optoelectronic devices.

In some embodiments, the optoelectronic device may be selected from an organic light emitting diode (OLED), an organic photovoltaic cell (OPV), an organic light emitting electrochemical cell (OLEEC), an organic field effect transistor (OFET), an organic light emitting field effect transistor, an organic laser, an organic spintronic device, an organic sensor, or an organic plasmon emitting diode (OPED).

In yet another aspect, the present disclosure further provides an optoelectronic device comprising a compound, a mixture, or an organic functional film as described herein.

In some embodiments, the optoelectronic device may be selected from an organic light emitting diode (OLED), an organic photovoltaic cell (OPV), an organic light emitting electrochemical cell (OLEEC), an organic field effect transistor (OFET), an organic light emitting field effect transistor, an organic laser, an organic spintronic device, an organic sensor, or an organic plasmon emitting diode (OPED).

Preferably, the optoelectronic device is an electroluminescent device, such as an organic light emitting diode (OLED), an organic light emitting electrochemical cell (OLEEC), an organic light emitting field effect transistor, a perovskite light emitting diode (PeLED), and a quantum dot light emitting diode (QD-LED), where one of the functional layers comprises a compound, or a mixture, or an organic functional film as described herein. The functional layer may be selected from a hole-injection layer, a hole-transport layer, an electron-injection layer, an electron-transport layer, a light-emitting layer, or a cathode passivation layer (CPL).

In some embodiments, the optoelectronic device is an electroluminescent device, comprising two electrodes, and the functional layer is located on the same side of the two electrodes.

In some embodiments, the optoelectronic device comprises a light emitting unit and a color conversion layer, where the color conversion layer comprises a compound, or a mixture, or an organic functional film as described herein.

In some embodiments, the light emitting unit is selected from a solid-state light emitting device. The solid-state light emitting device is preferably selected from a LED, an organic light emitting diode (OLED), an organic light emitting electrochemical cell (OLEEC), an organic light emitting field effect transistor, a perovskite light emitting diode (PeLED), a quantum dot light emitting diode (QD-LED), or a nanorod LED (see DOI: 10.1038/srep28312).

In some embodiments, the light emitting unit emits blue light, which is converted into green light by the color conversion layer.

In some embodiments, the light emitting unit emits green light, which is converted into yellow or red light by the color conversion layer.

In yet another aspect, the present disclosure further provides a display comprising at least three pixels of red, green and blue, where the blue pixel comprises a blue emitting unit, and the pixel of red or green comprises a blue emitting unit and a corresponding red or green color conversion layer.

In yet another aspect, the present disclosure further provides an organic light-emitting device comprising a substrate, a first electrode, an organic light-emitting layer, a second electrode, a color conversion layer, and an encapsulation layer (e.g., an outermost encapsulation layer) in sequence from bottom to top, the second electrode is at least partially transparent, where 1) the color conversion layer comprises a compound as described herein and an emitter E2; 2) the color conversion layer can at least partially absorbs the light emitted by the organic light-emitting layer through the second electrode; 3) the emission spectrum of the compound is on the short wavelength side of the absorption spectrum of the emitter E2, and at least partially overlaps with the absorption spectrum of the emitter E2; 4) the FWHM of the emission spectrum of the emitter E2 ≤55 nm.

The compound, the emitter E2, and the embodiments thereof are as described above.

In some embodiments, the color conversion layer can absorb 30% or more of the light emitted by the organic light-emitting layer through the second electrode, preferably 40% or more, and most preferably 45% or more.

In some embodiments, the color conversion layer can absorb 90% or more of the light emitted by the organic light-emitting layer through the second electrode, preferably 95% or more, more preferably 99% or more, and most preferably 99.9% or more.

In some embodiments, the thickness of the color conversion layer is between 100 nm and 5 μm, preferably between 150 nm and 4 μm, more preferably between 200 nm and 3 μm, and most preferably between 200 nm and 2 μm.

In some embodiments, the organic light-emitting device is an OLED. More preferably, the first electrode is an anode, and the second electrode is a cathode. Particularly preferably, the organic light-emitting device is a top emission OLED.

The substrate should be opaque or transparent. A transparent substrate could be used to produce a transparent light-emitting device (for example: Bulovic et al., Nature 1996, 380, p 29, and Gu et al., Appl. Phys. Lett. 1996, 68, p 2606). The substrate can be rigid/flexible, e.g. it can be plastic, metal, semiconductor wafer, or glass. Preferably, the substrate has a smooth surface. Particularly desirable are substrates without surface defects. In some embodiments, the substrate is flexible and can be selected from a polymer film or plastic with a glass transition temperature (Tg) >150° C., preferably >200° C., more preferably >250° C., and most preferably >300° C. Examples of the suitable flexible substrate includes poly ethylene terephthalate (PET) and polyethylene glycol (2,6-naphthalene) (PEN).

The choice of anodes may include a conductive metal, or a metal oxide, or a conductive polymer. The anode should be able to easily inject holes into a hole-injection layer (HIL), a hole-transport layer (HTL), or a light-emitting layer. In some embodiments, the absolute value of the difference between the work function of the anode and the HOMO energy level of the emitter of the light-emitting layer, or the HOMO energy level/valence band energy level of the p-type semiconductor materials of the hole-injection layer (HIL)/hole-transport layer (HTL)/electron-blocking layer (EBL) <0.5 eV, preferably <0.3 eV, more preferably <0.2 eV. Examples of anode materials may include, but not limited to: Al, Cu, Au, Ag, Mg, Fe, Co, Ni, Mn, Pd, Pt, ITO, aluminum-doped zinc oxide (AZO), etc. Other suitable anode materials are known and can be readily selected for use by the general technicians in this field. The anode materials can be deposited using any suitable technique, such as a suitable physical vapor deposition method, including RF magnetron sputtering, vacuum thermal evaporation, e-beam, etc. In some embodiments, the anode is patterned. Patterned conductive ITO substrates are commercially available and can be used to produce the devices as used herein.

The choice of cathode may include a conductive metal or a metal oxide. The cathode should be able to easily inject electrons into the EIL, the ETL, or the directly into the light-emitting layer. In some embodiments, the absolute value of the difference between the work function of the cathode and the LUMO energy level of the emitter of the light-emitting layer, or the LUMO energy level/conduction band energy level of the n-type semiconductor materials of the electron-injection layer (EIL)/electron-transport layer (ETL)/hole-blocking layer (HBL) <0.5 eV, preferably <0.3 eV, and most preferably <0.2 eV. In principle, all materials that can be used as cathodes for OLEDs may be applied as cathode materials for the devices as used herein. Examples of cathode materials include, but not limited to: Al, Au, Ag, Ca, Ba, Mg, LiF/Al, MgAg alloys, BaF₂/Al, Cu, Fe, Co, Ni, Mn, Pd, Pt, ITO, etc. The cathode materials can be deposited using any suitable technique, such as the suitable physical vapor deposition method, including RF magnetron sputtering, vacuum thermal evaporation, e-beam, etc. In some embodiments, the transmittance of the cathode in the range of 400-680 nm ≥40%, preferably ≥45%, more preferably ≥50%, and most preferably ≥60%. Typically, 10-20 nm of Mg:Ag alloys can be used as transparent cathodes, and the ratio of the Mg:Ag can range from 2:8 to 0.5:9.5.

The light-emitting layer of the organic light-emitting device preferably comprises a blue fluorescent host and a blue fluorescent dopant. In some embodiments, the light-emitting layer comprises a blue phosphorescent host and a blue phosphorescent dopant. The OLED may also comprise other functional layers, such as a hole-injection layer (HIL), a hole-transport layer (HTL), an electron-blocking layer (EBL), an electron-injection layer (EIL), an electron-transport layer (ETL), or a hole-blocking layer (HBL). Materials suitable for use in these functional layers are described in detail above and in WO2010135519A1, US 20090134784A1, and WO2011110277A1, the contents of these three documents are hereby incorporated herein for reference.

Further, the organic light-emitting device further comprises a cathode capping layer (CPL).

In some embodiments, the CPL is disposed between the second electrode and the color conversion layer.

In some embodiments, the CPL is disposed on the top of the color conversion layer.

The CPL material generally requires high refractive index (n), such as n≥1.95 @460 nm, n≥1.90@520 nm, n≥1.85@620 nm. Examples of the CPL materials include:

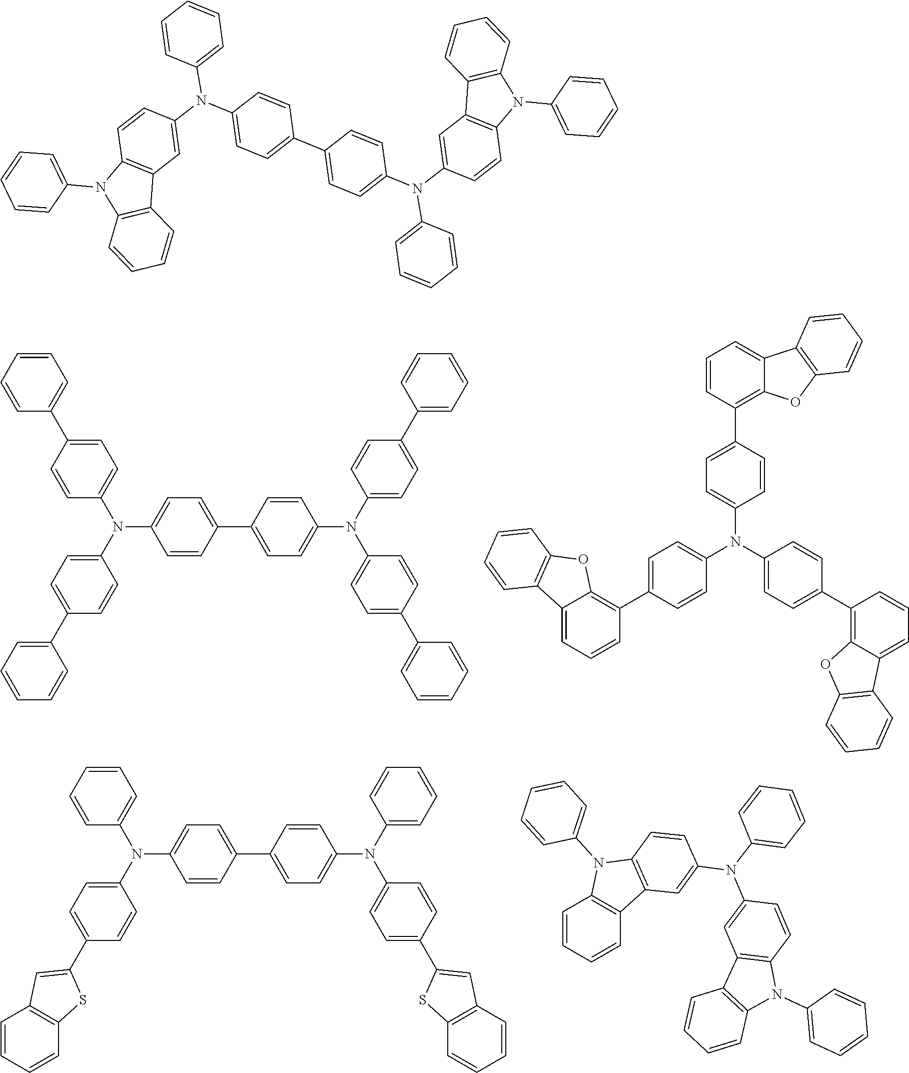 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc2c(c1)oc1c(-c3ccc(N(c4ccc(-c5cccc6c5oc5ccccc56)cc4)c4ccc(-c5cccc6c5oc5ccccc56)cc4)cc3)cccc12 ; c1ccc(N(c2ccc(-c3ccc(N(c4ccccc4)c4ccc(-c5cc6ccccc6s5)cc4)cc3)cc2)c2ccc(-c3cc4ccccc4s3)cc2)cc1 ; c1ccc(N(c2ccc3c(c2)c2ccccc2n3-c2ccccc2)c2ccc3c(c2)c2ccccc2n3-c2ccccc2)cc1]

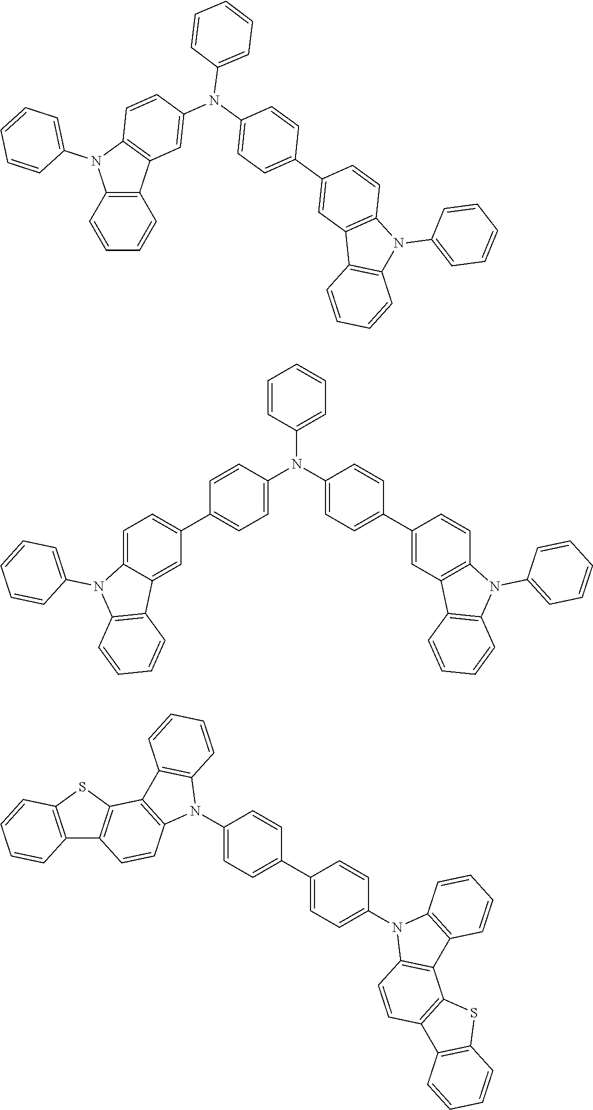 [SMILES: c1ccc(N(c2ccc(-c3ccc4c(c3)c3ccccc3n4-c3ccccc3)cc2)c2ccc3c(c2)c2ccccc2n3-c2ccccc2)cc1 ; c1ccc(N(c2ccc(-c3ccc4c(c3)c3ccccc3n4-c3ccccc3)cc2)c2ccc(-c3ccc4c(c3)c3ccccc3n4-c3ccccc3)cc2)cc1 ; c1ccc2c(c1)sc1c2ccc2c1c1ccccc1n2-c1ccc(-c2ccc(-n3c4ccccc4c4c5sc6ccccc6c5ccc43)cc2)cc1]

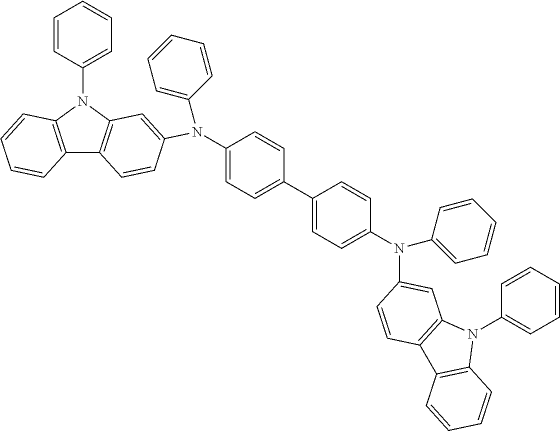 [SMILES: c1ccc(N(c2ccc(-c3ccc(N(c4ccccc4)c4ccc5c6ccccc6n(-c6ccccc6)c5c4)cc3)cc2)c2ccc3c4ccccc4n(-c4ccccc4)c3c2)cc1]

More further examples of the CPL materials can be found in the following patent literatures: KR20140128653A, KR20140137231A, KR20140142021A, KR20140142923A, KR20140143618A, KR20140145370A, KR20150004099A, KR20150012835A, U.S. Pat. No. 9,496,520B2, US2015069350A1, CN103828485B, CN104380842B, CN105576143A, TW201506128A, CN103996794A, CN103996795A, CN104744450A, CN104752619A, CN101944570A, US2016308162A1, U.S. Pat. No. 9,095,033B2, US2014034942A1, WO2017014357A1; the above patent documents are incorporated herein by reference in their entirety.

In some embodiments, the color conversion layer comprises a above-mentioned CPL material. In some embodiments, the color conversion layer is formed by co-evaporated by one above-mentioned CPL material, the compound (i.e., the host material H) and the emitter E2 as described herein. In some embodiments, the mass ratio of the compound (i.e., the host material H) is in the range of 20% to 50%, and the mass ratio of the emitter E2 is in the range of 10% to 15%.

Preferably, the encapsulation layer of the organic light-emitting device is thin-film encapsulated (TFE).

In yet another aspect, the present disclosure further provides a display panel, where at least one pixel comprises an organic light-emitting device as described herein.

The present disclosure will be described below in conjunction with the preferred embodiments, but the present disclosure is not limited to the following embodiments. It should be understood that the scope of the present disclosure is covered by the scope of the claims of the present disclosure, and those skilled in the art should understand that certain changes may be made to the embodiments of the present disclosure.

### SPECIFIC EMBODIMENT

### Examples of Compound Synthesis

### Example 1: The Synthetic Route of the Compound 1 is Shown as Follows

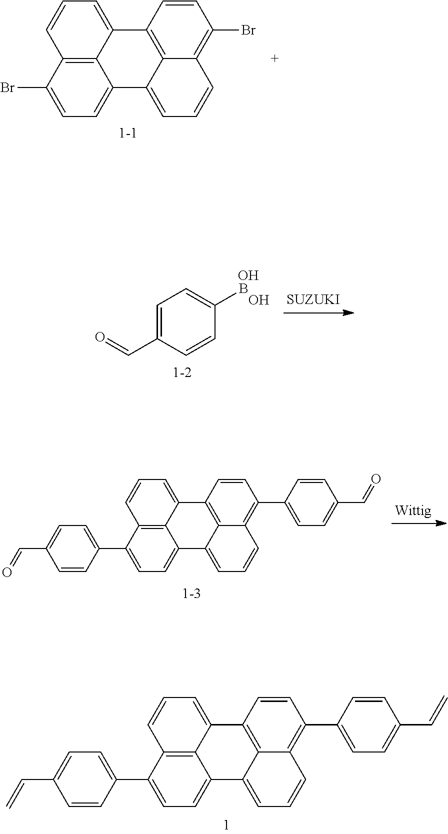

Intermediate 1-3 was synthesize via the classical SUZUKI coupling reaction, and the procedure is as follows: 10.00 mmol of intermediate 1-1, 20.08 mmol of intermediate 1-2, and 20.00 mmol of potassium carbonate were added in turn to a 500 mL three-necked flask under nitrogen atmosphere. After adding 200 mL of toluene, 0.3 mol of catalyst Pd(PPh₃)₄ was added under stirring, and the mixture was heated to reflux. Monitored by TLC, after the reaction was completed, the reaction solution was cooled to room temperature, washed with water and the combined aqueous phase was extracted with dichloromethane three times. After that, the organic phase was combined, dried over anhydrous Na₂SO₄, filtered, and the solvent was removed to obtain the crude product. The residue was purified by flash column chromatography to yield 7.54 mmol (75.4% yield) of intermediate 1-3, and further vacuum-dried for use. MS (ASAP)=460.2.

Compound 1 was synthesized via the classical Wittig reaction and the procedure is as follows: 1.0 mmol of KOH was dissolved in DMSO, and 10.00 mmol of intermediates 1-3 was added into the above solution under nitrogen atmosphere. After stirring at room temperature for 1 h, 10.05 mmol of methylphosphonolysine DMSO solution was added dropwise to the above mixture at 0° C. After the addition, the mixture was stirred at 0° C. for another 4 h. After the reaction was completed, the reaction was quenched with water, and extracted with trichloromethane three times, then the organic phase was dried over anhydrous Na₂SO₄, filtered, and the solvent was removed to obtain the crude product. After that, the residue was purified by flash column chromatography to yield 8.34 mmol (83.4% yield) of compound 1, and further vacuum-dried for use. MS (ASAP)=456.6.

### Example 2: The Synthetic Route of the Compound 2 is Shown as Follows

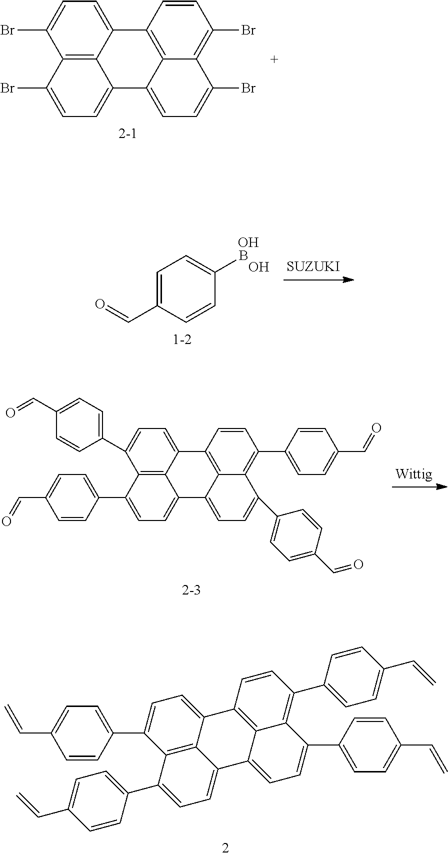

Synthesis of intermediate 2-3: The synthetic method was similar to the synthetic method of the intermediate 1-3, using the classical SUZUKI coupling reaction, yield: 80.4%. Vacuum-dried for use. MS (ASAP)=668.2.

Synthesis of compound 2: The synthetic method was similar to the synthetic method of the compound 1, using the classical Wittig reaction, yield: 82.6%. MS (ASAP)=660.3.

### Example 3: The Synthetic Route of the Compound 3 is Shown as Follows

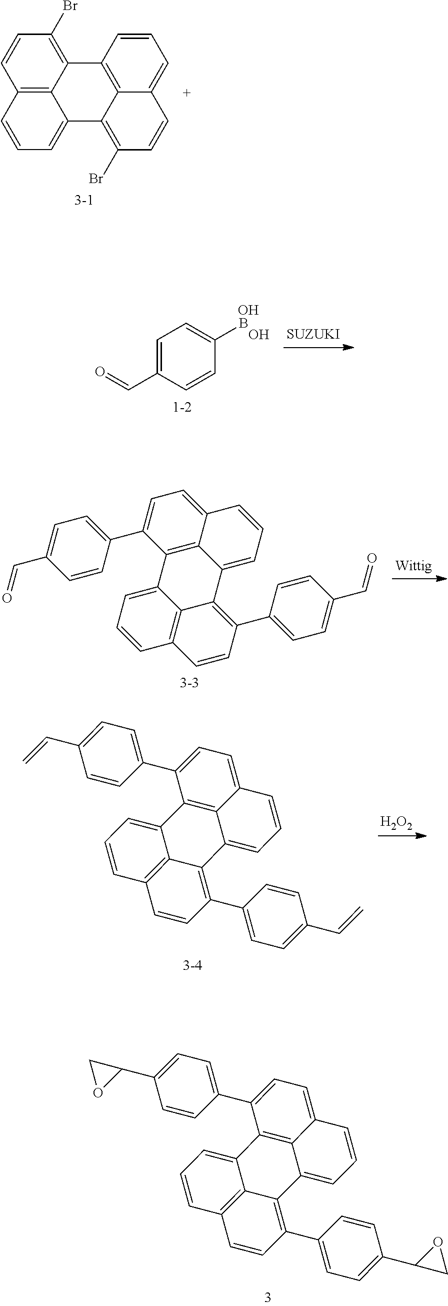

Synthesis of intermediate 3-3: The synthetic method was similar to the synthetic method of the intermediate 1-3, using the classical SUZUKI coupling reaction, yield: 74.3%. Vacuum-dried for use. MS (ASAP)=460.2.

Synthesis of intermediate 3-4: The synthetic method was similar to the synthetic method of the compound 1, using the classical Wittig reaction, yield: 81.3%. Vacuum-dried for use. MS (ASAP)=456.3.

Synthesis of compound 3: 10.00 mmol of intermediate 3-4 was dissolved in dichloromethane under nitrogen atmosphere, then 20.5 mmol of H₂O₂ solution was added to the above solution and the resulting mixture was stirred at room temperature for 2 h. After the reaction was completed, the reaction was quenched with a large amount of dichloromethane and H₂O. After the separation, the organic phase was dried over anhydrous Na₂SO₄, filtered, and the organic solvent was removed to obtain 8.88 mmol (88.8% yield) of compound 3. MS (ASAP)=488.20.

### Example 4: The Synthetic Route of the Compound 4 is Shown as Follows

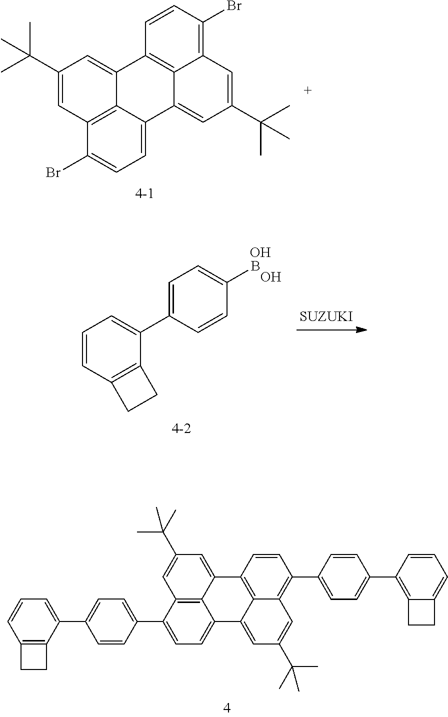

Synthesis of compound 4: Synthesis was carried out similarly to that of Compound 1 using the classical SUZUKI coupling reaction, Yield: 68.4%. Vacuum dried. MS (ASAP)=720.3.

### Example 5: The Synthetic Route of the Compound 5 is Shown as Follows

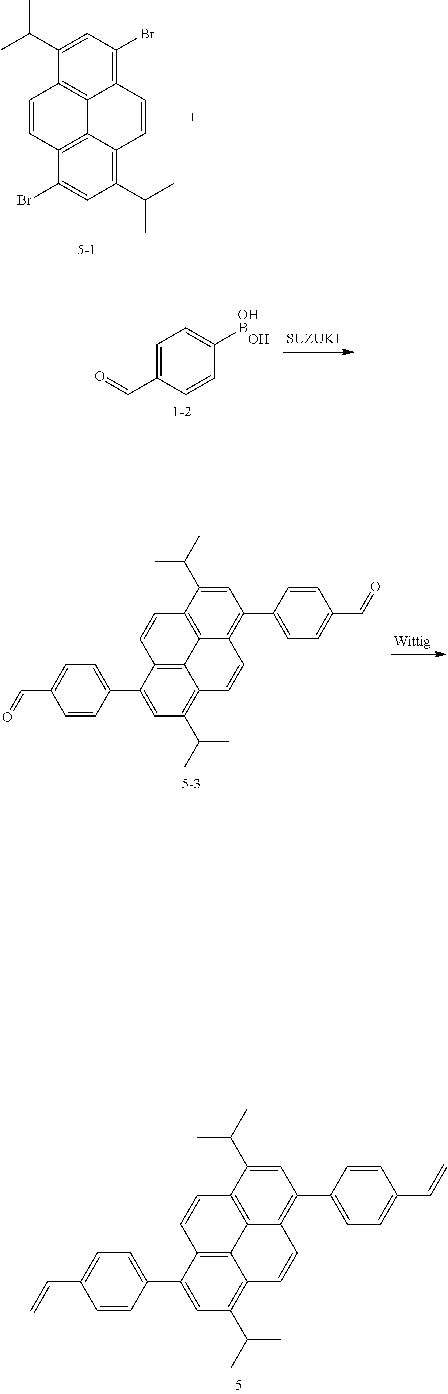

Synthesis of intermediate 5-3: The synthetic method was similar to the synthetic method of the intermediate 1-3, using the classical SUZUKI coupling reaction, yield: 78.9%. Vacuum-dried for use. MS (ASAP)=494.2.

Synthesis of compound 5: The synthetic method was similar to the synthetic method of the compound 1, using the classical Wittig reaction, yield: 83.5%. MS (ASAP)=490.3.

### Example 6: The Synthetic Route of the Compound 6 is Shown as Follows

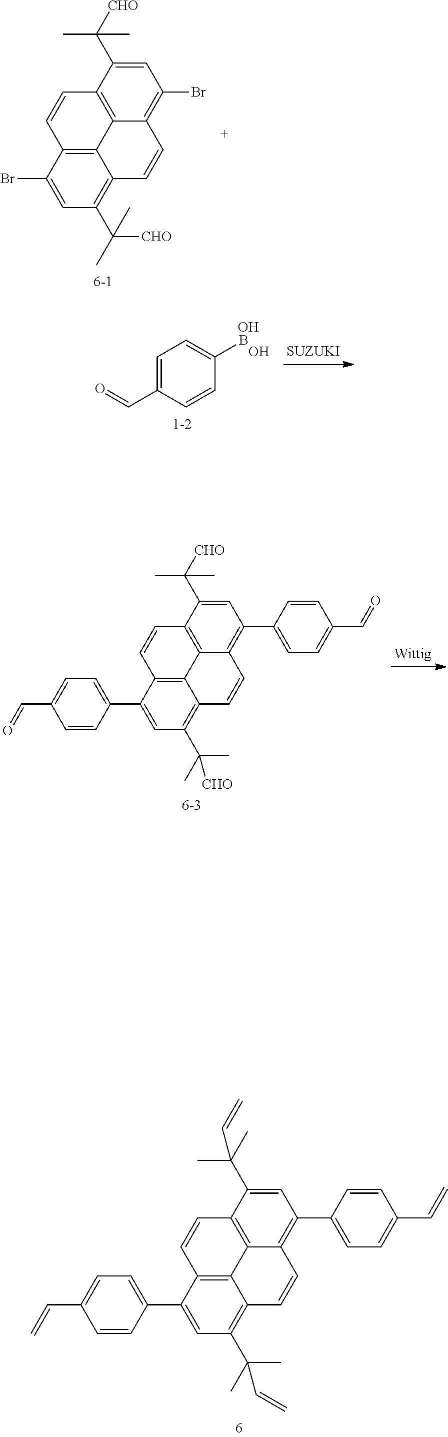

Synthesis of intermediate 6-3: The synthetic method was similar to the synthetic method of the intermediate 1-3, using the classical SUZUKI coupling reaction, yield: 75.8%. Vacuum-dried for use. MS (ASAP)=550.2.

Synthesis of compound 6: The synthetic method was similar to the synthetic method of the compound 1, using the classical Wittig reaction, yield: 84.4%. MS (ASAP)=542.3.

### Example 7: The Synthetic Route of the Compound 7 is Shown as Follows

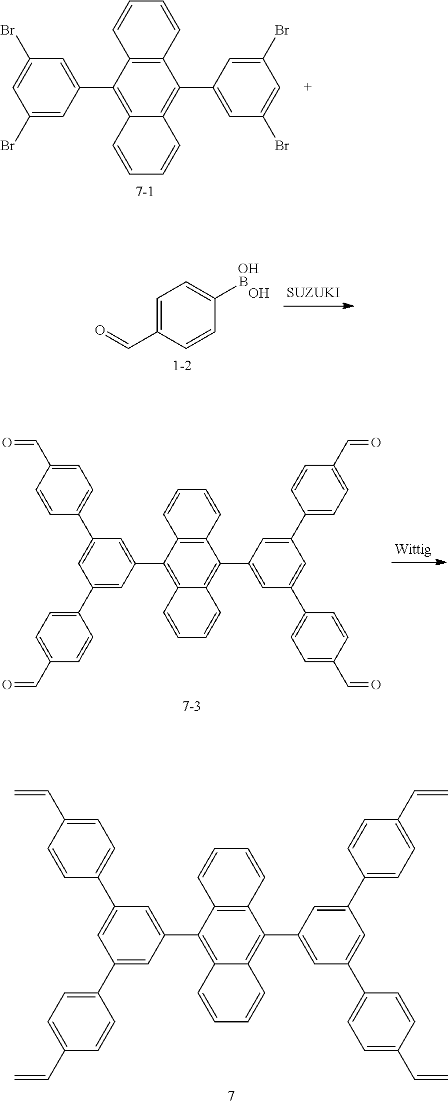

Synthesis of intermediate 7-3: The synthetic method was similar to the synthetic method of the intermediate 1-3, using the classical SUZUKI coupling reaction, yield: 79.3%. Vacuum-dried for use. MS (ASAP)=746.3.

Synthesis of compound 7: The synthetic method was similar to the synthetic method of the compound 1, using the classical Wittig reaction, yield: 74.4%. MS (ASAP)=738.3.

### Example 8: The Synthetic Route of the Compound 8 is Shown as Follows

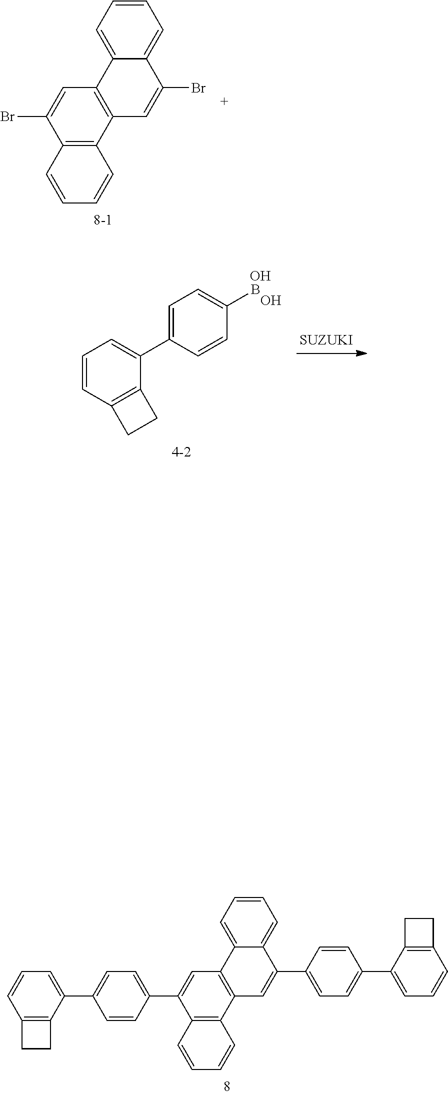

Synthesis of compound 8: The synthetic method was similar to the synthetic method of the compound 4, using the classical SUZUKI coupling reaction, yield: 80.3%. Vacuum-dried for use. MS (ASAP)=584.3.

### Example 9: The Synthetic Route of the Compound 9 is Shown as Follows

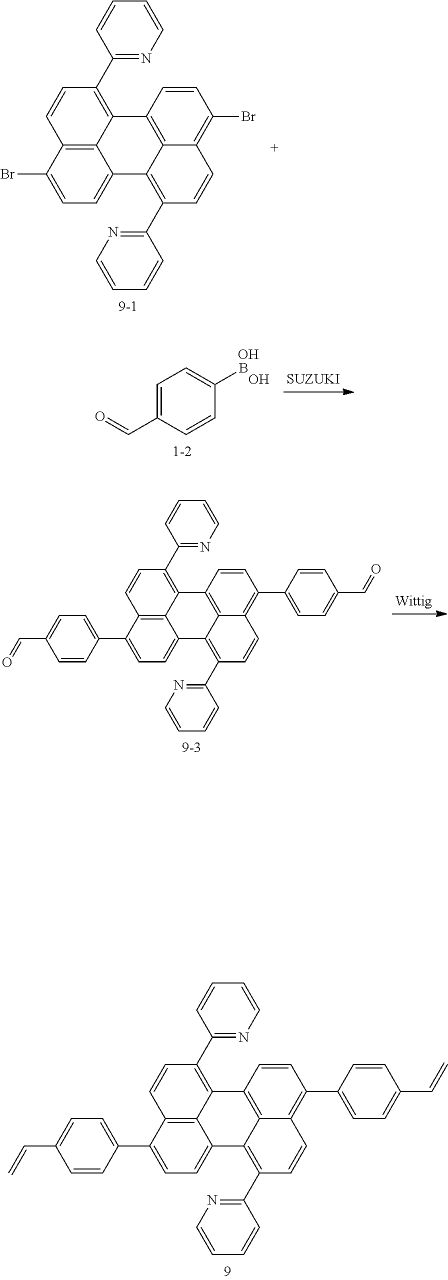

Synthesis of intermediate 9-3: The synthetic method was similar to the synthetic method of the intermediate 1-3, using the classical SUZUKI coupling reaction, yield: 78.4%. Vacuum-dried for use. MS (ASAP)=614.2.

Synthesis of compound 9: The synthetic method was similar to the synthetic method of the compound 1, using the classical Wittig reaction, yield: 77.6%. MS (ASAP)=610.3.

### Example 10: The Synthetic Route of the Compound 10 is Shown as Follows

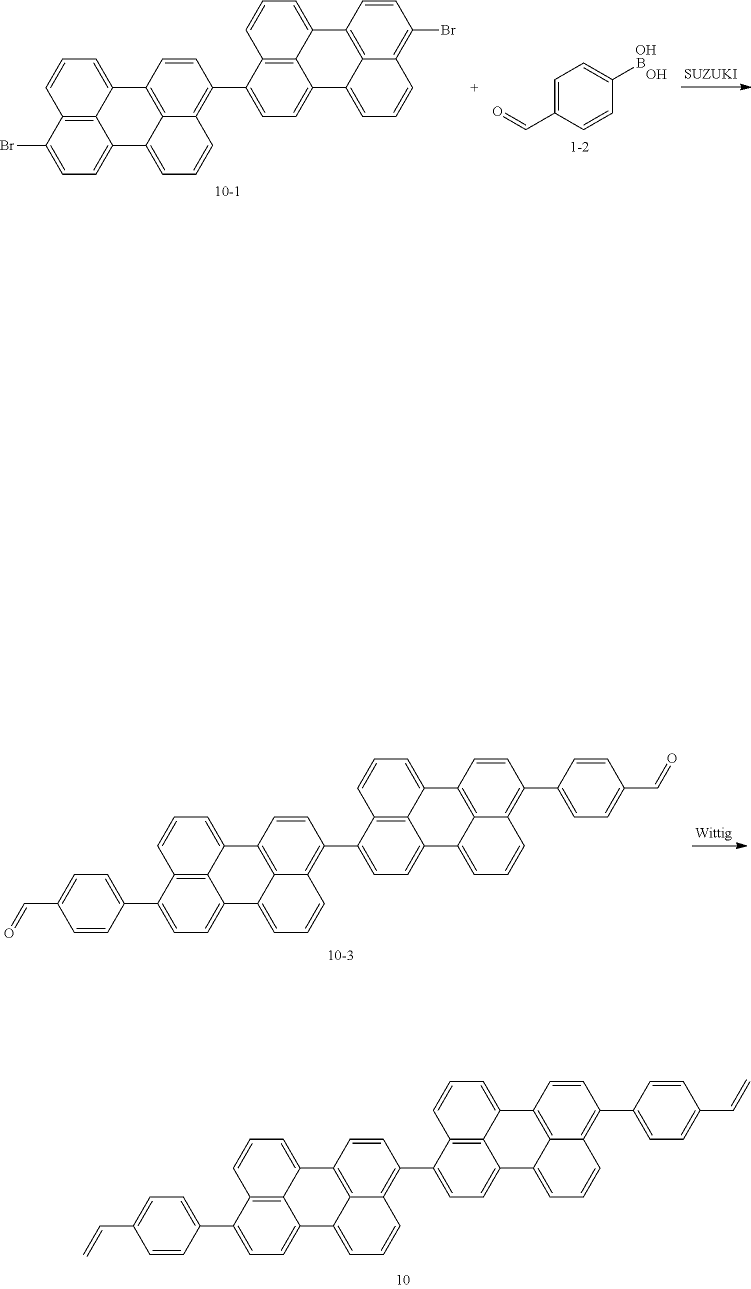

Synthesis of intermediate 10-3: The synthetic method was similar to the synthetic method of the intermediate 1-3, using the classical SUZUKI coupling reaction, yield: 79.8%. Vacuum-dried for use. MS (ASAP)=710.2.

Synthesis of compound 10: The synthetic method was similar to the synthetic method of the compound 1, using the classical Wittig reaction, yield: 74.7%. MS (ASAP)=706.3.

### Example 11: The Synthetic Route of the Compound 11 is Shown as Follows

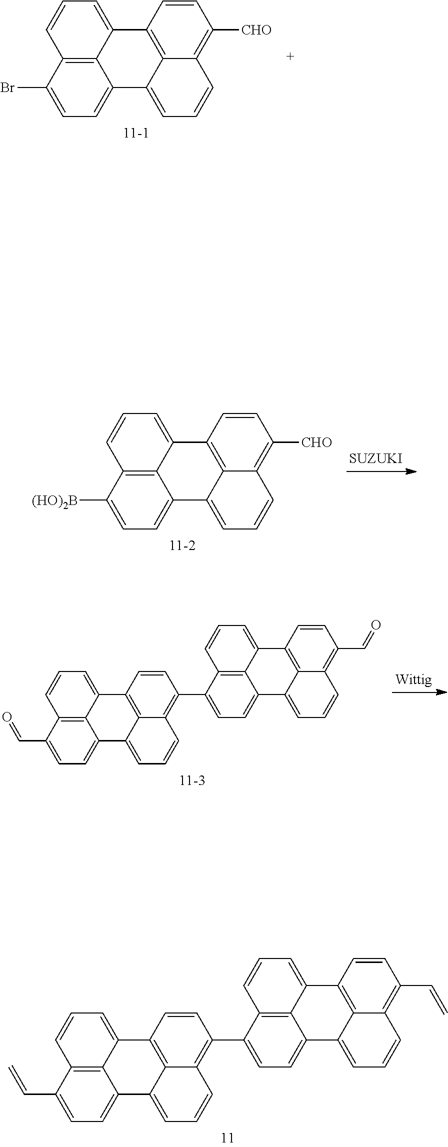

Synthesis of intermediate 11-3: The synthetic method was similar to the synthetic method of the intermediate 1-3, using the classical SUZUKI coupling reaction, yield: 81.8%. Vacuum-dried for use. MS (ASAP)=558.2.

Synthesis of Compound 11: The synthetic method was similar to the synthetic method of the compound 1, using the classical Wittig reaction, yield: 80.7%. MS (ASAP)=554.2.

### Example 12: The Synthetic Route of the Compound 12 is Shown as Follows

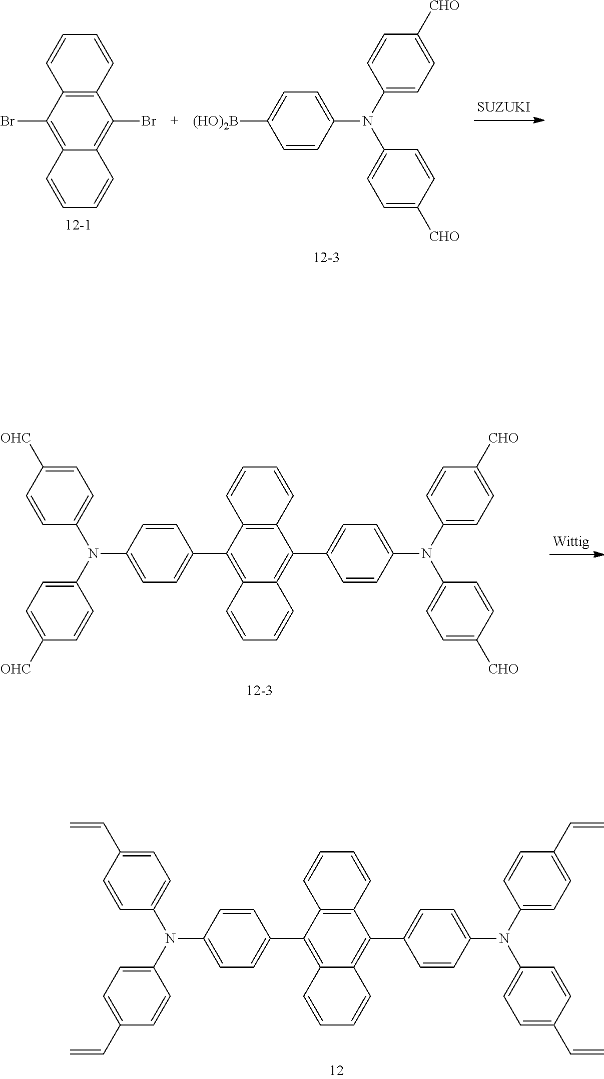 [SMILES: Brc1c2ccccc2c(Br)c2ccccc12 ; O=Cc1ccc(N(c2ccc(C=O)cc2)c2ccc(B(O)O)cc2)cc1 ; O=Cc1ccc(N(c2ccc(C=O)cc2)c2ccc(-c3c4ccccc4c(-c4ccc(N(c5ccc(C=O)cc5)c5ccc(C=O)cc5)cc4)c4ccccc34)cc2)cc1 ; C=Cc1ccc(N(c2ccc(C=C)cc2)c2ccc(-c3c4ccccc4c(-c4ccc(N(c5ccc(C=C)cc5)c5ccc(C=C)cc5)cc4)c4ccccc34)cc2)cc1]

Synthesis of intermediate 12-3: The synthetic method was similar to the synthetic method of the intermediate 1-3, using the classical SUZUKI coupling reaction, yield: 85.8%. Vacuum-dried for use. MS (ASAP)=776.3.

Synthesis of compound 12: The synthetic method was similar to the synthetic method of the compound 1, using the classical Wittig reaction, yield: 81.2%. MS (ASAP)=768.4.

### Example 13: The Synthetic Route of the Compound 13 is Shown as Follows

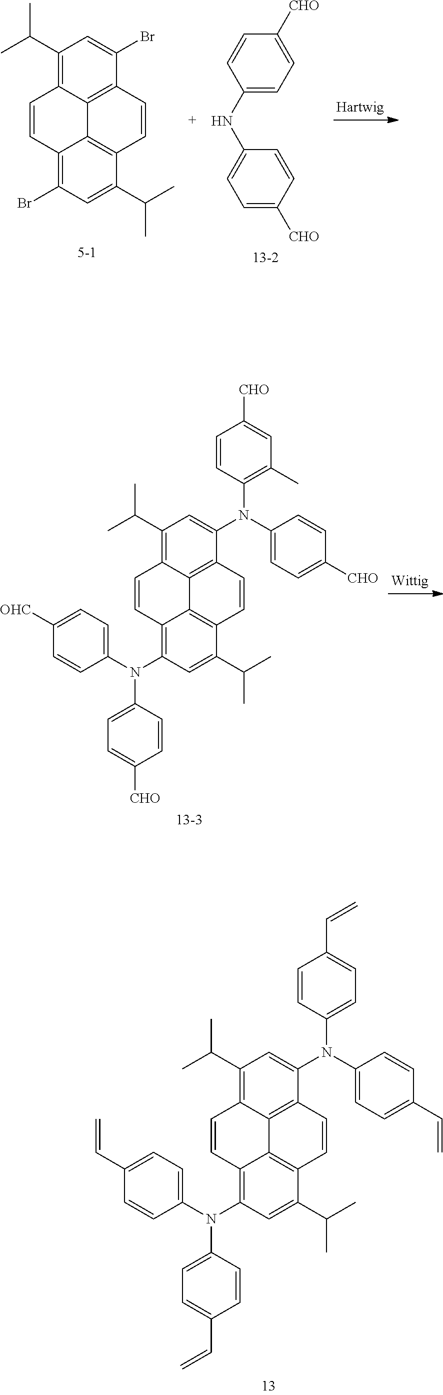 [SMILES: CC(C)c1cc(Br)c2ccc3c(C(C)C)cc(Br)c4ccc1c2c43 ; O=Cc1ccc(Nc2ccc(C=O)cc2)cc1 ; Cc1cc(C=O)ccc1N(c1ccc(C=O)cc1)c1cc(C(C)C)c2ccc3c(N(c4ccc(C=O)cc4)c4ccc(C=O)cc4)cc(C(C)C)c4ccc1c2c43 ; C=Cc1ccc(N(c2ccc(C=C)cc2)c2cc(C(C)C)c3ccc4c(N(c5ccc(C=C)cc5)c5ccc(C=C)cc5)cc(C(C)C)c5ccc2c3c54)cc1]

Synthesis of Intermediate 13-3:

Intermediate 13-3 was synthesised via the classical Buchwald-Hartwig reaction and the procedure is as follows: 10.00 mmol of intermediate 13-1, 20.08 mmol of intermediate 13-2, and 20.00 mmol of potassium carbonate were added in turn to a 500 mL three-necked flask under nitrogen protection. After adding 200 mL of toluene, 0.30 mol of the catalyst Pd(OAc)₂ was added under stirring, along with 0.30 mmol of tri-tert-butylphosphorus, the mixture was heated to reflux. Monitored by TLC, after the reaction was completed, the reaction solution was cooled to room temperature, washed with water and the combined aqueous phase was extracted with dichloromethane three times. After that, the organic phase was combined, dried over anhydrous Na₂SO₄, filtered, and the solvent was removed to obtain the crude product. The residue was purified by flash column chromatography to yield 6.24 mmol (62.4% yield) of intermediate 13-3, and further vacuum-dried for use. MS (ASAP)=732.3.

Synthesis of compound 13: The synthetic method was similar to the synthetic method of the compound 1, using the classical Wittig reaction, yield: 82.5%. MS (ASAP)=724.4.

### Example 14: The Synthetic Route of the Compound 14 is Shown as Follows

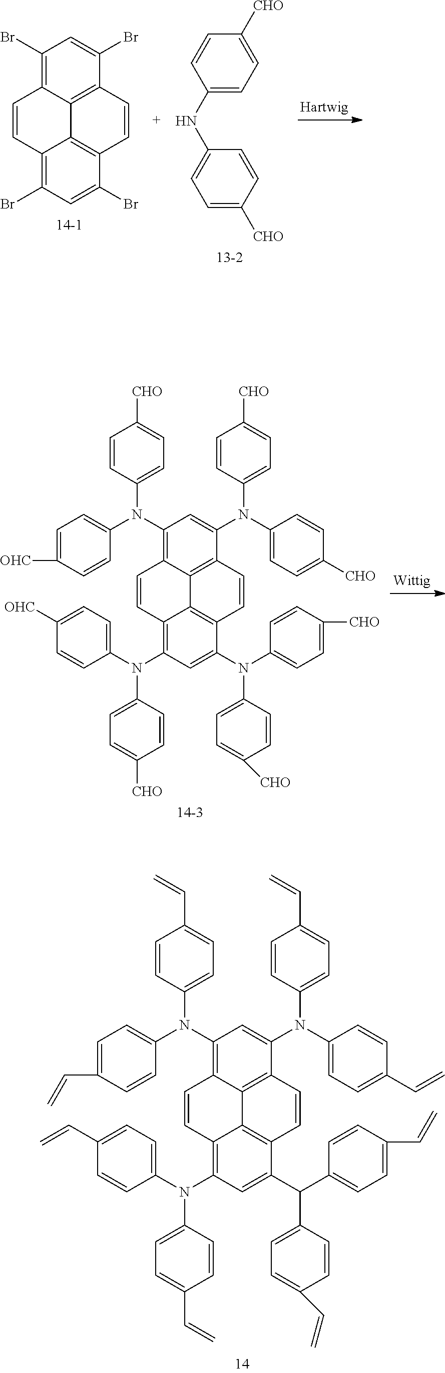 [SMILES: Brc1cc(Br)c2ccc3c(Br)cc(Br)c4ccc1c2c43 ; O=Cc1ccc(Nc2ccc(C=O)cc2)cc1 ; O=Cc1ccc(N(c2ccc(C=O)cc2)c2cc(N(c3ccc(C=O)cc3)c3ccc(C=O)cc3)c3ccc4c(N(c5ccc(C=O)cc5)c5ccc(C=O)cc5)cc(N(c5ccc(C=O)cc5)c5ccc(C=O)cc5)c5ccc2c3c54)cc1 ; C=Cc1ccc(C(c2ccc(C=C)cc2)c2cc(N(c3ccc(C=C)cc3)c3ccc(C=C)cc3)c3ccc4c(N(c5ccc(C=C)cc5)c5ccc(C=C)cc5)cc(N(c5ccc(C=C)cc5)c5ccc(C=C)cc5)c5ccc2c3c54)cc1]

Synthesis of intermediate 14-3: The synthetic method was similar to the synthetic method of the intermediate 13-3, using the classical SUZUKI coupling reaction, yield: 55.8%. Vacuum-dried for use. MS (ASAP)=1094.3.

Synthesis of compound 14: The synthetic method was similar to the synthetic method of the compound 1, using the classical Wittig reaction, yield: 82.8%. Vacuum-dried for use. MS (ASAP)=1078.5.

### Example 15: The Synthetic Route of the Compound 15 is Shown as Follows

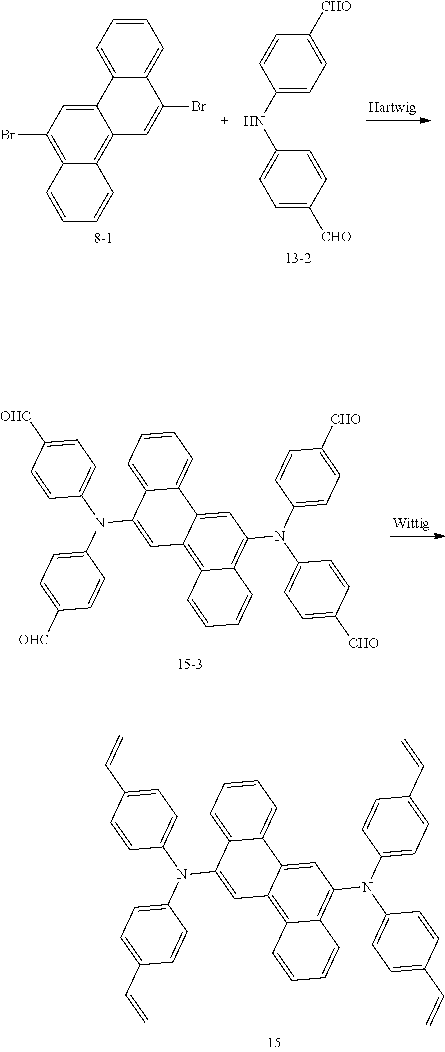

Synthesis of intermediate 15-3: The synthetic method was similar to the synthetic method of the intermediate 13-3, using the classical SUZUKI coupling reaction, yield: 66.8%. Vacuum-dried for use. MS (ASAP).

Synthesis of compound 15: The synthetic method was similar to the synthetic method of the compound 1, using the classical Wittig reaction, yield: 84.5%. Vacuum-dried for use. MS (ASAP)=666.4.

The dopant materials used in the present disclosure are as follows:

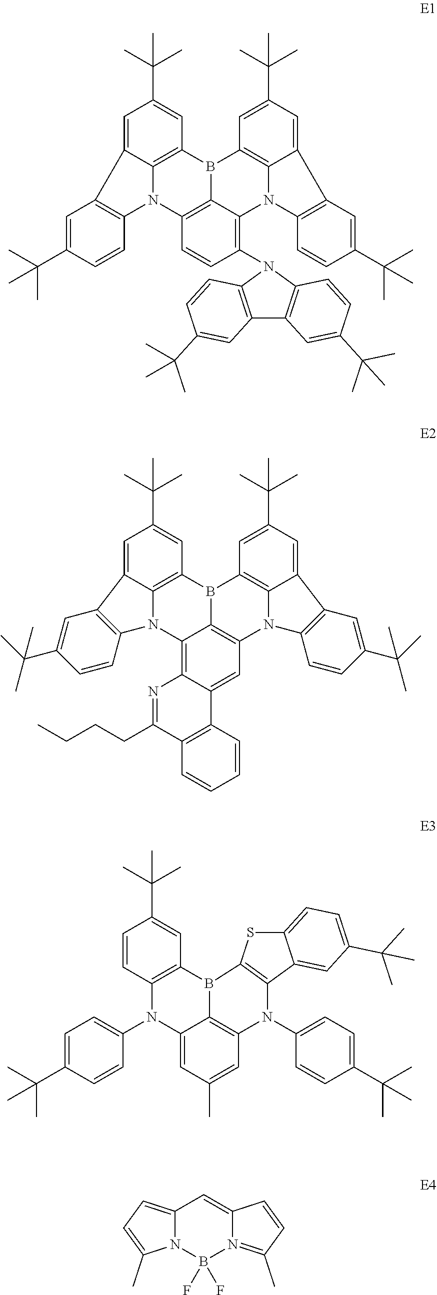

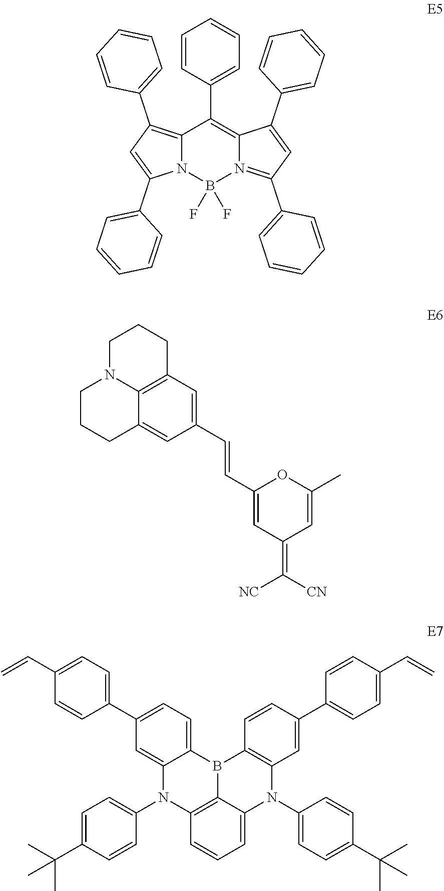

As a green light emitter, E1 was synthesized with reference to Angew. Chem. Int. Ed. 10.1002/anie.202007210. As a green light emitter, E2 was synthesized with reference to Angew. Chem. Int. Ed. 10.1002/anie.202008264. As a blue light emitter, E3 was synthesized with reference to US2020395553A1. E4 is a green light emitter, both E5 and E6 are red light emitters. As a blue light emitter, E7 was synthesized with referred to patent application No. CN202110370910.9.

Optical Performance Testing

The absorption and emission spectrum of the compound 1-compound 15 were measured in toluene, and the molar extinction coefficient (ε) was calculated according to the corresponding concentration. The εs of the compound 1-compound 15 ≥2*10⁴; among them, the εs of the compounds 5-8, 15≥5*10⁴; and the εs of the compounds 1, 2, 3, 4, 10, 11, 12, 13, and 14≥1*10⁵. In addition, the absorption and emission spectrums of the E1-E7 were measured in toluene; each of E1-E5, and E7 has a narrow emission spectrum, with a FWHM of less than 40 nm.

Preparation of Polymer-Containing Formulations and Organic Functional Films

100 mg of polymethyl methacrylate (PMMA), 50 mg of the host material (i.e., compound 1-compound 15) for color conversion, and 5 mg of the emitter (i.e., the dopant material (E1-E7) for color conversion) were dissolved in 1 mL of n-butyl acetate to obtain a clear solution (i.e., a formulation or a printing ink). Using a KW-4a spin coater, the above clear solution was spun-coated on the surface of the quartz glass to form an uniform thin film, which is an organic functional film (i.e., a color conversion film). When the above-mentioned color conversion film is thinner than 4 μm, the optical density (OD) thereof can reach 3 or more. In particular, for the color conversion layer shown in Table 1, when the thickness is thinner than 3 μm, the optical density (OD) thereof can reach 3 or more, and the FWHM thereof 40 nm.

TABLE 1
Optimal material combinations for color conversion layers

| color conversion | | | |
|---|---|---|---|
| layer(Ink) | host | dopant | color |
| 1 | compound 1 | E1 | green |
| 2 | compound 3 | E1 | green |
| 3 | compound 4 | E1 | green |
| 4 | compound 10 | E1 | green |
| 5 | compound 11 | E1 | green |
| 6 | compound 12 | E1 | green |
| 7 | compound 13 | E1 | green |
| 8 | compound 14 | E1 | green |
| 9 | compound 15 | E1 | green |
| 10 | compound 1 | E2 | green |
| 11 | compound 3 | E2 | green |
| 12 | compound 4 | E2 | green |
| 13 | compound 10 | E2 | green |
| 14 | compound 11 | E2 | green |
| 15 | compound 12 | E2 | green |
| 16 | compound 13 | E2 | green |
| 17 | compound 14 | E2 | green |
| 18 | compound 15 | E2 | green |
| 19 | compound 1 | E4 | green |
| 20 | compound 3 | E4 | green |
| 21 | compound 4 | E4 | green |
| 22 | compound 10 | E4 | green |
| 23 | compound 11 | E4 | green |
| 24 | compound 12 | E4 | green |
| 25 | compound 13 | E4 | green |
| 26 | compound 14 | E4 | green |
| 27 | compound 15 | E4 | green |
| 28 | compound 5 | E3 | blue |
| 29 | compound 6 | E3 | blue |
| 30 | compound 7 | E3 | blue |
| 31 | compound 8 | E3 | blue |
| 32 | compound 5 | E7 | blue |
| 33 | compound 6 | E7 | blue |
| 34 | compound 7 | E7 | blue |
| 35 | compound 8 | E7 | blue |
| 36 | compound 1 | E5 | red |
| 37 | compound 3 | E5 | red |
| 38 | compound 4 | E5 | red |
| 39 | compound 10 | E5 | red |
| 40 | compound 11 | E5 | red |
| 41 | compound 12 | E5 | red |
| 42 | compound 13 | E5 | red |
| 43 | compound 14 | E5 | red |
| 44 | compound 15 | E5 | red |

Preparation of Resin Prepolymer-Containing Formulations and Organic Functional Films

The resin prepolymers-containing formulations and organic functional films could be obtained that the host and dopant materials for color conversion were premixed with a resin prepolymer such as methyl methacrylate, styrene, or methylstyrene. Initiated by 1-5 wt % of a photoinitiator (such as TPO (diphenyl (2,4,6-trimethylbenzoyl) phosphine oxide, 97%, CAS: 75980-60-8)), the obtained formulation could form a thin film by spin-coating, coating method, etc., and the obtained film was further cured by irradiation with a 365 nm or 390 nm UV LED lamp to form a color conversion film.

The blue color conversion film can be disposed on top of a UV or deep blue self-emitting unit that exhibits deep blue emission in the range of 330 nm to 450 nm; through the blue color converter, the deep blue light could be converted to the blue light ranging from 450 nm to 500 nm.

The green color conversion film can be disposed on the top of a blue self-emitting unit that exhibits blue emission in the range of 450 nm to 500 nm; through the green color converter, the blue light could be converted to green or yellow light ranging from 500 nm to 580 nm.

The red color conversion film can be disposed on the top of a blue or green self-emitting unit that exhibits blue or green emission in the range of 450 nm to 550 nm; through the red color converter, the blue or green light could be converted to red light ranging from 580 nm to 650 nm.

Preparation of Top Emission OLED Devices

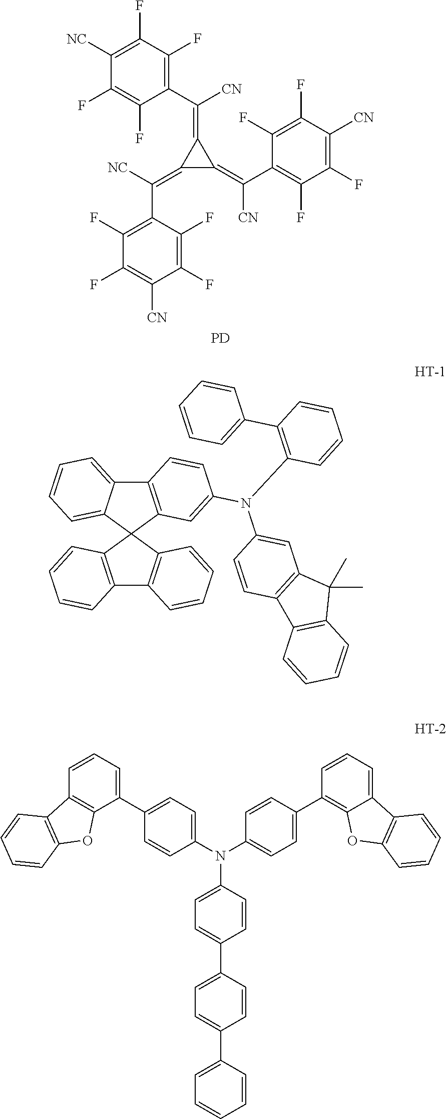

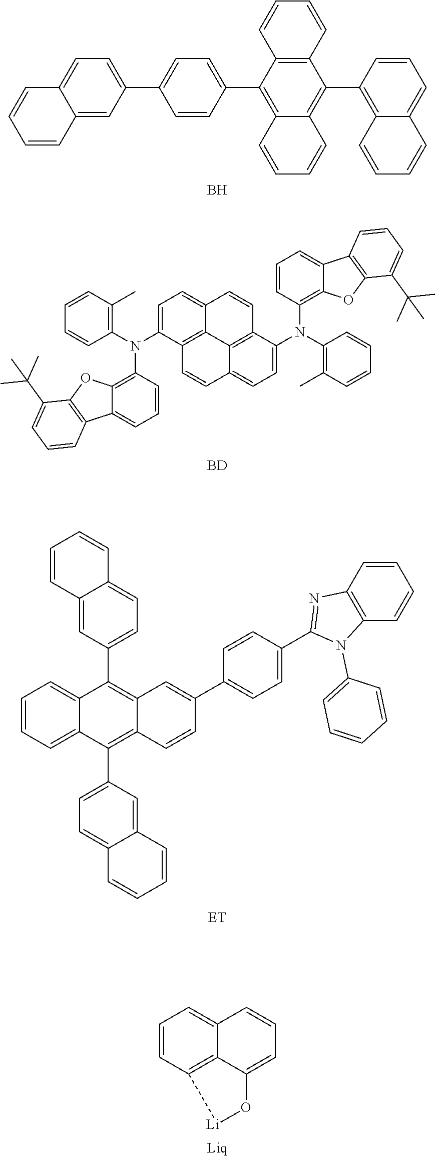

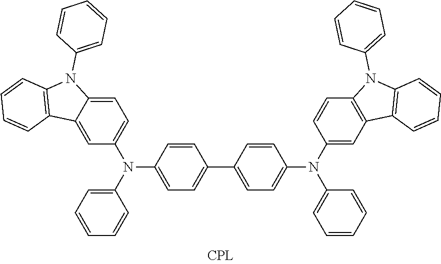 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1]

Preparation of Resin-Containing Color Conversion Material Ink:

Preparation of Prepolymer:

n-butyl acetate (42 wt %), methyl methacrylate (MMA) (50 wt %), hydroxypropyl acrylate (HPA) (3 wt %), and biphenyl peroxide (BPO) (5 wt %) were respectively weighed, mixed, and stirred at 125° C. for 50 minutes to obtain a prepolymer; the obtained prepolymer (67 wt %), n-butyl acetate (30 wt %), the host (i.e., compound 1-compound 15) (2.5 wt %) and the dopant (i.e., E1-E7) (0.5 wt %) material for color conversion were stirred to obtain a clear solution, which was filtered to obtain a Ink; the corresponding Ink1-Ink44 were obtained according to the material combinations in Table 1.

Preparation of Resin-Free Color Conversion Material Ink:

The host (i.e., compound 1-compound 15) (2.5 wt %) and the dopant (i.e., E1-E7) (0.5 wt %) material for color conversion were dissolved in n-butyl acetate, stirred to obtain a clear solution, and filtered to obtain a Ink; the corresponding Ink1b-Ink44b were obtained according to the material combinations in Table 1.

1. Preparation of Green Light-Emitting Device 1:

a. Cleaning of Ag-containing ITO (indium tin oxide) top substrate: the substrate was ultrasonically cleaned with strip liquid, pure water, and isopropyl alcohol in sequence, then treated with ozone in argon after drying.

b. Evaporation: the resultant substrate was mounted on a vacuum deposition apparatus in high vacuum (1×10⁻⁶ mbar), the weight ratio of PD and HT-1 was controlled to be 3:100 to form a hole-injection layer (HIL) having a thickness of 10 nm, followed by evaporation of compound HT-1 on the hole-injection layer to form a hole-transport layer (HTL) having a thickness of 120 nm, and then immediately followed by evaporation of compound HT-2 on the hole-transport layer to form a hole—buffer layer having a thickness of 10 nm. Then BH and BD at a weight ratio of 100:3 formed a light-emitting layer film having a thickness of 25 nm. Subsequently, ET and Liq were respectively placed in two different evaporation sources, and co-deposited on the light-emitting layer at a weight ratio of 50:50 to form an electron-transport layer having a thickness of 35 nm. Yb was then deposited on the electron-transport layer to form an electron-injection layer having a thickness of 1.5 nm, and Mg:Ag (1:9) alloy was deposited on the electron-injection layer to form a cathode having a thickness of 16 nm.

c. On the cathode, Ink (i.e., Ink1-Ink27) was printed with Haisi electronic IJDAS310 (nozzle FUJIFILM Dimatix DMC-11610), then cured by irradiation with a 390 nm UV LED lamp to obtain a color conversion layer with a thickness of about 3 μm.

d. Encapsulation: encapsulating the device in a nitrogen-regulated glove box with UV curable resin.

2. Preparation of Green Light-Emitting Device 1b:

Steps a, b, and d are the same as described in the procedure for preparing the above-mentioned green light-emitting device 1, and the step c is as follows.

c. On the cathode, Ink (i.e., Ink1b-Ink27b) was printed with Haisi electronic IJDAS310 (nozzle FUJIFILM Dimatix DMC-11610) to obtain a color conversion layer with a thickness of 1 μm to 2 μm.

3. Preparation of Blue Light-Emitting Device 1:

Steps a and d are the same as described in the procedure for preparing the above-mentioned green light-emitting device 1, and steps b and c are as follows.

b. Evaporation: the resultant substrate was mounted on a vacuum deposition apparatus in high vacuum (1×10⁻⁶ mbar), the weight ratio of PD and HT-1 was controlled to be 3:100 to form a hole-injection layer (HIL) having a thickness of 10 nm, followed by evaporation of compound HT-1 on the hole-injection layer to form a hole-transport layer (HTL) having a thickness of 120 nm, and then immediately followed by evaporation of compound HT-2 on the hole-transport layer to form a hole-buffer layer having a thickness of 10 nm. Then BH (100%) formed a light-emitting layer film having a thickness of 25 nm. Subsequently, ET and Liq were placed in two different evaporation sources, and co-deposited on the light-emitting layer at a weight ratio of 50:50 to form an electron-transport layer having a thickness of 35 nm. Yb was then deposited on the electron-transport layer to form an electron-injection layer having a thickness of 1.5 nm, and Mg:Ag (1:9) alloy was deposited on the electron-injection layer to form a cathode having a thickness of 16 nm.

c. On the cathode, Ink (i.e., Ink28-Ink35) was printed with Haisi electronic IJDAS310 (nozzle FUJIFILM Dimatix DMC-11610) to obtain a color conversion layer with a thickness of about 3 μm.

4. Preparation of Blue Light-Emitting Device 1b:

Steps a, b, and d are the same as described in the procedure for preparing the above-mentioned blue light-emitting device 1, and the step c is as follows.

c. On the cathode, Ink (i.e., Ink28b-Ink35b) was printed with Haisi electronic IJDAS310 (nozzle FUJIFILM Dima DMC-11610) to obtain a color conversion layer with a thickness of 1 μm to 2 μm.

5. Preparation of Green Light-Emitting Device 2:

Steps a, b, and c are the same as described in the procedure for preparing the above-mentioned green light-emitting device 1, and the steps d and e are as follows:

d. CPL with a thickness of 70 nm was evaporated on the color conversion layer and used as an optical capping layer.

e. Encapsulation: encapsulating the device in a nitrogen-regulated glove box with UV curable resin.

6. Preparation of Red Light-Emitting Device 1:

Steps a, b, and d are the same as described in the procedure for preparing the above-mentioned green light-emitting device 1, and the step c is as follows:

c. On the cathode, Ink (i.e., Ink36-Ink44) was printed with Haisi electronic IJDAS310 (nozzle FUJIFILM Dimatix DMC-11610), then cured by irradiation with a 390 nm UV LED lamp to obtain a color conversion layer with a thickness of 2 μm to 3 μm.

7. Preparation of the Red Light-Emitting Device 1b:

Steps a, b, and d are the same as described in the procedure for preparing the above-mentioned green light-emitting device 1, and the step c is as follows:

c. On the cathode, Ink (i.e., Ink36b-Ink44b) was printed with Haisi electronic IJDAS310 (nozzle FUJIFILM Dimatix DMC-11610) to obtain a color conversion layer with a thickness of 1 μm to 2 μm.

All of the above light-emitting devices have high color purity, and the FWHMs of their emission spectrums are below 40 nm.

The technical features of the above-described embodiments can be combined in any ways. For the sake of brevity, not all possible combinations of the technical features of the above-described embodiments have been described. However, as long as there are no contradictions in the combination of these technical features, they should be considered to be within the scope of this specification.

What described above are several embodiments of the present disclosure, and they are specific and in detail, but not intended to limit the scope of the present disclosure. It will be understood that improvements can be made without departing from the concept of the present disclosure, and all these modifications and improvements are within the scope of the present disclosure. The scope of the present disclosure shall be subject to the appended claims.

What is claimed is:

1. A compound, comprising a structural unit of one of formulas (1)-(4), 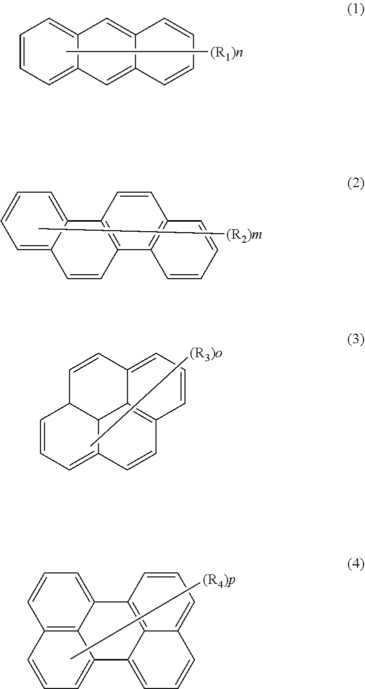
wherein, R₁ to R₄ are substituents and are independently selected from a group consisting of a C₁-C₂₀ linear alkyl group, a C₁-C₂₀ linear haloalkyl group, a C₁-C₂₀ linear alkoxy group, a C₁-C₂₀ linear thioalkoxy group, a C₃-C₂₀ branched/cyclic alkyl group, a C₃-C₂₀ branched/cyclic haloalkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ substituted ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —NO₂, —CF₃, —Cl, —Br, —F, —I, a cross-linkable group, a substituted/unsubstituted aromatic or heteroaromatic group containing 5 to 40 ring atoms, an aryloxy or heteroaryloxy group containing 5 to 40 ring atoms, an arylamine or heteroarylamine group containing 5 to 40 ring atoms, a disubstituted unit in any position of the above substituents, and any combination thereof;
n, o are integers from 1 to 10; m, p are integers from 1 to 12;
wherein, the compound comprises at least one cross-linkable group.

2. The compound according to claim 1, wherein the compound comprises a structural unit of formulas (1a)-(4a), (4b): 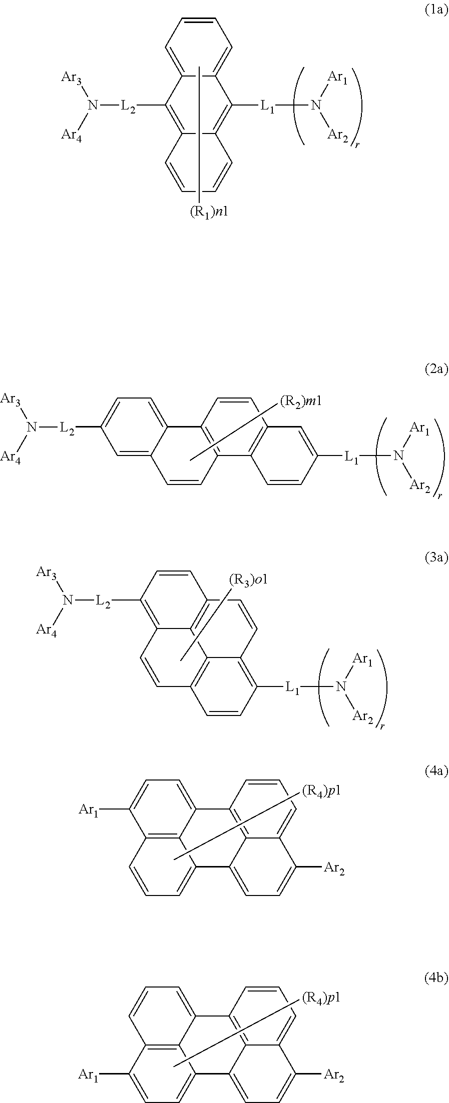
wherein:
n1, o1 are integers from 1 to 8; m1, p1 are integers from 1 to 10; r is 0 or 1;
R₁ to R₄ are the same as defined in claim 1;
each of Ar₁ to Ar₄ at each occurrence is independently selected from the group consisting of a substituted/unsubstituted aromatic or heteroaromatic group containing 5 to 40 ring atoms, an aryloxy or heteroaryloxy group containing 5 to 40 ring atoms, and any combination thereof;
each of L₁ and L₂ at each occurrence is independently selected from the group consisting of a single bond, a substituted/unsubstituted aromatic or heteroaromatic group containing 6 to 30 ring atoms.

3. The compound according to claim 1, wherein at least one of the cross-linkable group is selected from: a linear/cyclic alkenyl, a linear dienyl, a linear alkynyl; an enoxy, a dienoxy; an acrylic; a propylene oxide, an ethylene oxide; a silanyl; and a cyclobutanyl.

4. The compound according to claim 2, wherein at least one of the cross-linkable group is selected from: a linear/cyclic alkenyl, a linear dienyl, a linear alkynyl; an enoxy, a dienoxy; an acrylic; a propylene oxide, an ethylene oxide; a silanyl; and a cyclobutanyl.

5. The compound according to claim 1, wherein at least one of the cross-linkable group is selected from the following structures: 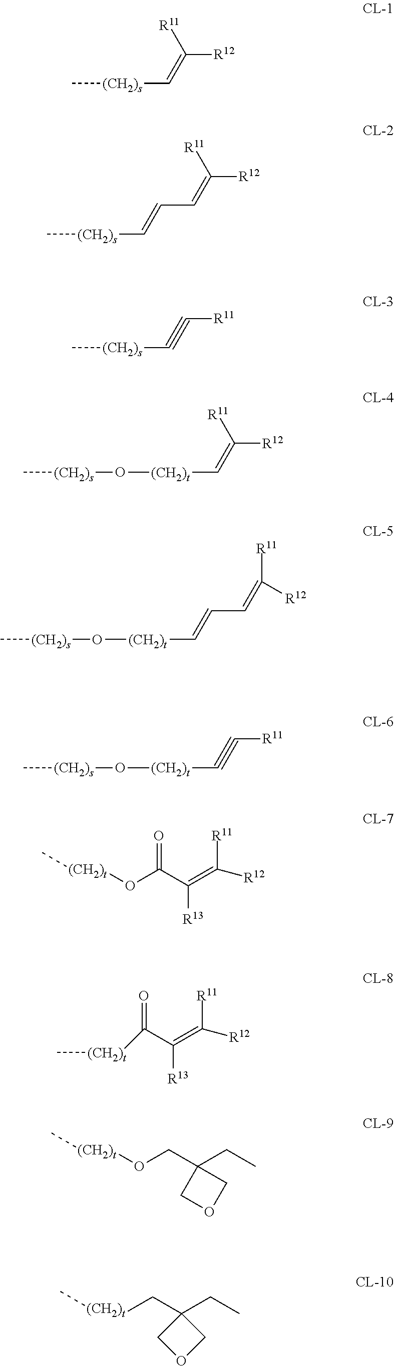 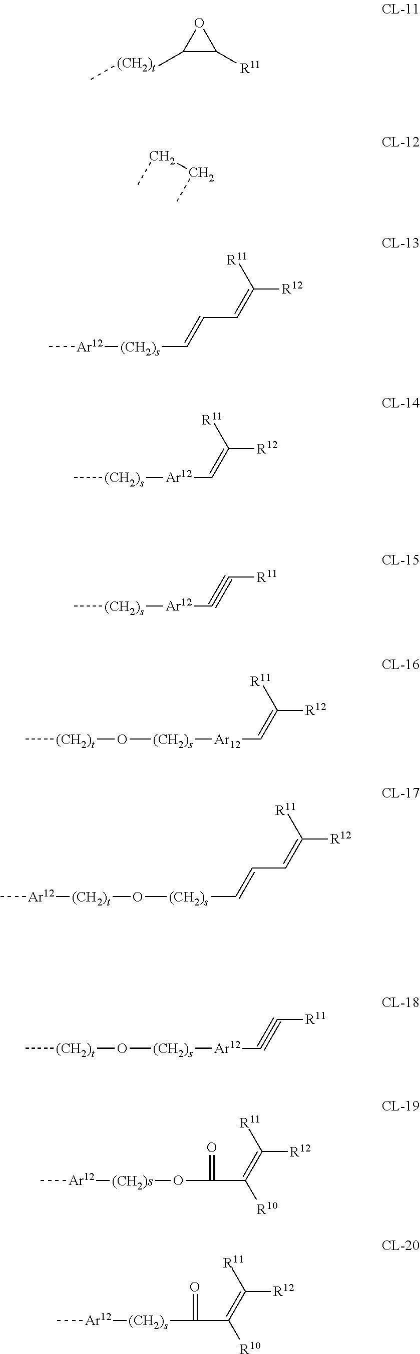 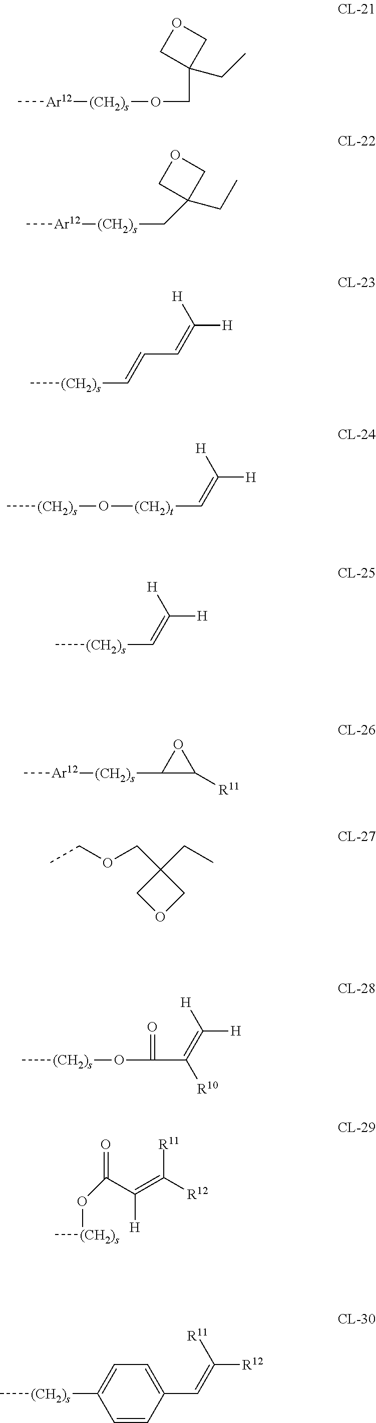 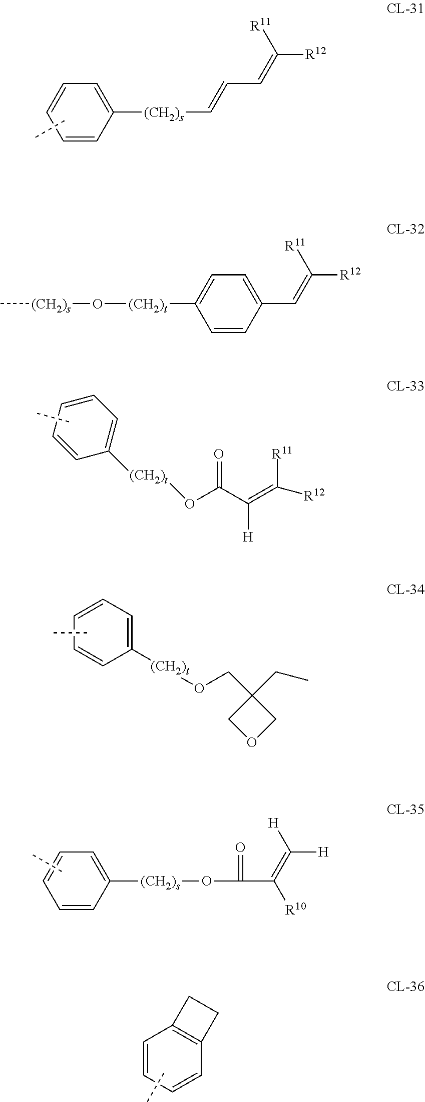
wherein: the dotted line represents a bonded bond, R¹⁰ to R¹³ are identically defined as the above-mentioned R₁ in claim 1, Ar¹² is defined as the above-mentioned Ar₁ in claim 1; s, t at each occurrence are integers >0.

6. The compound according to claim 2, wherein at least one of the cross-linkable group is selected from the following structures: 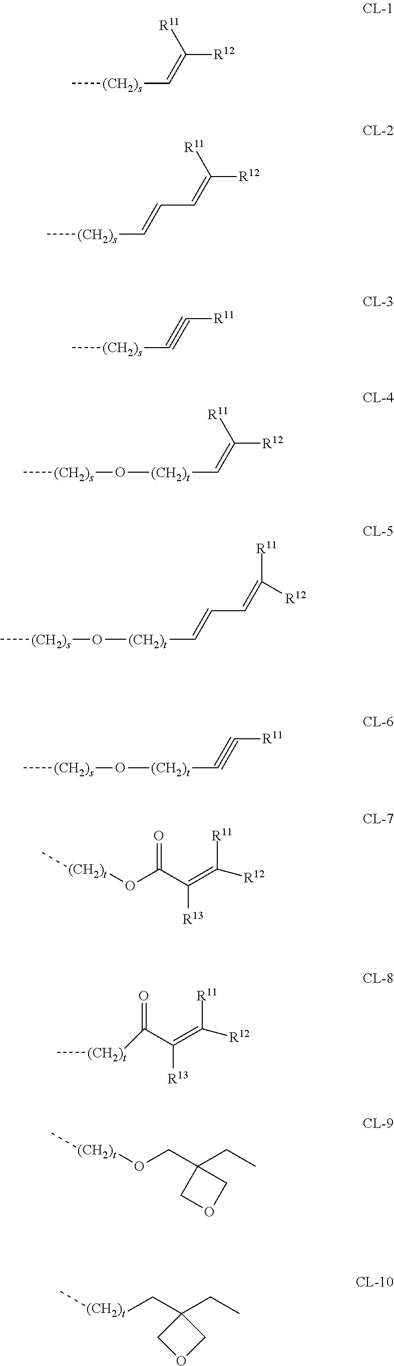 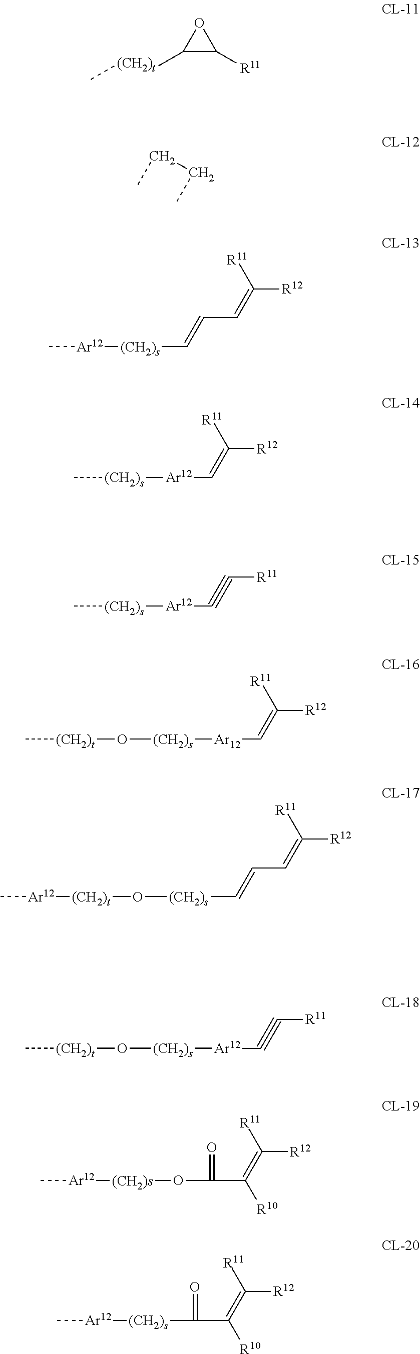 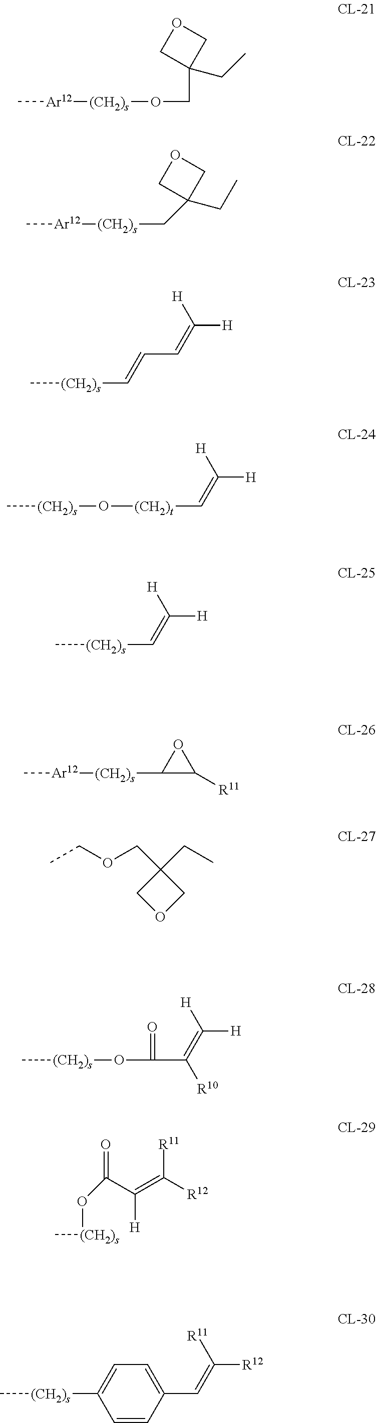 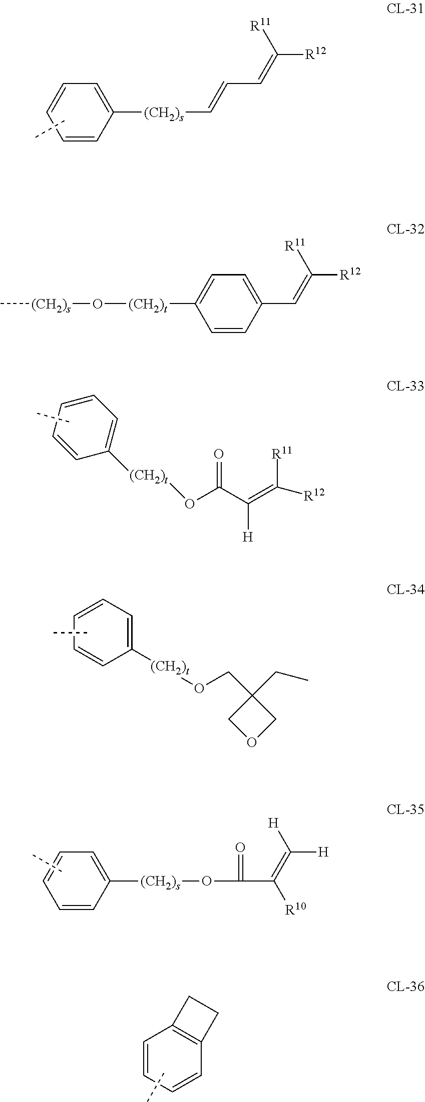
wherein: the dotted line represents a bonded bond, R¹⁰ to R¹³ are identically defined as the above-mentioned R₁ in claim 1, Ar¹² is defined as the above-mentioned Ar₁ in claim 1; s, t at each occurrence are integers >0.

7. The compound according to claim 3, wherein at least one of the cross-linkable group is selected from the following structures: 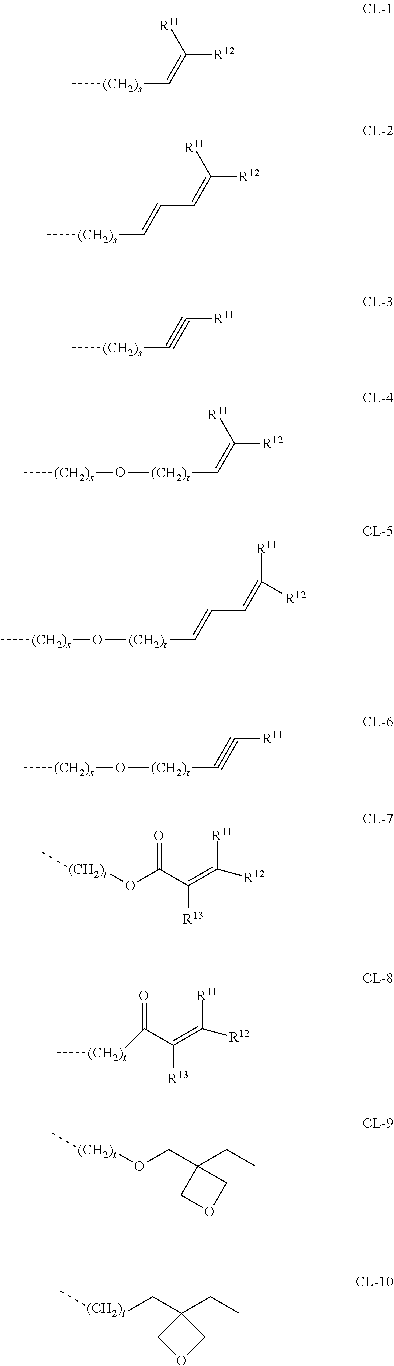 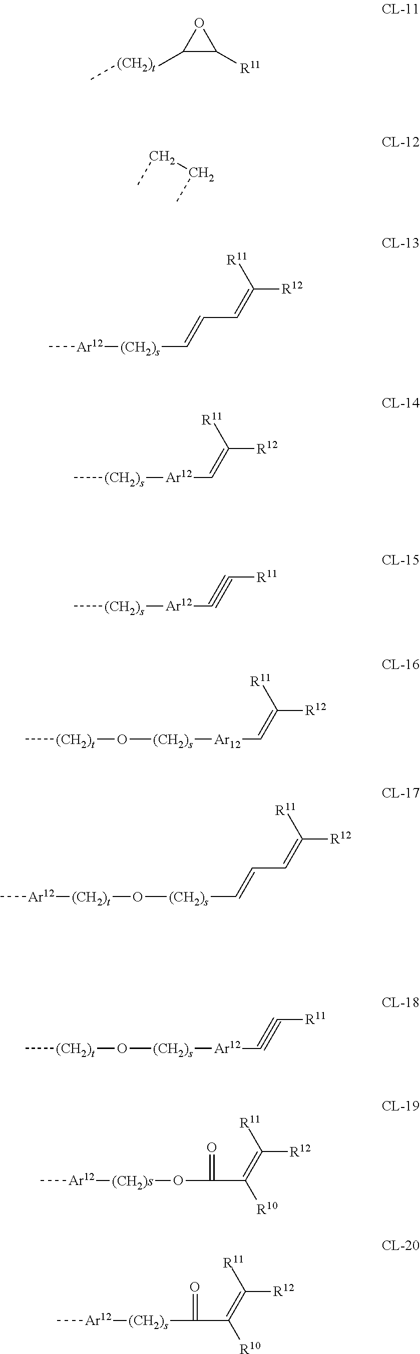 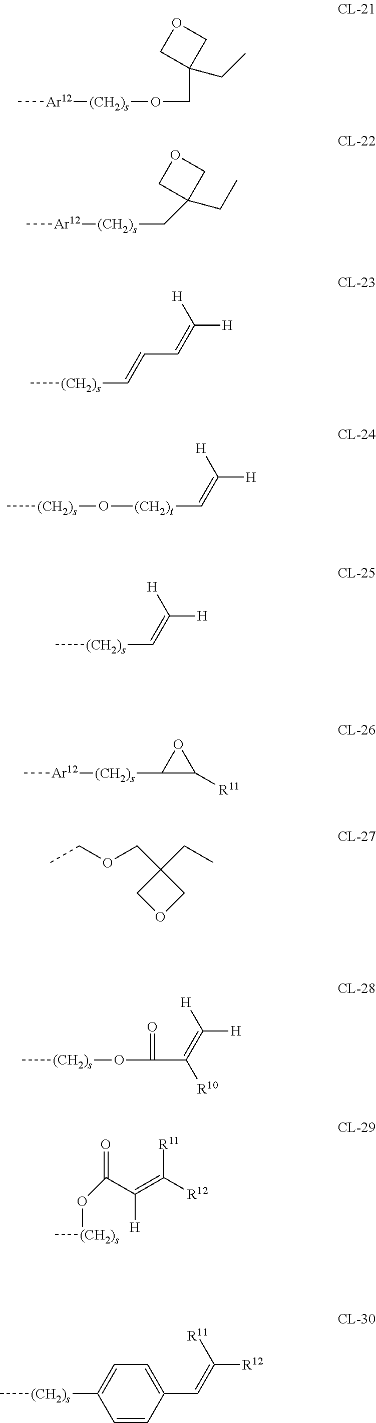 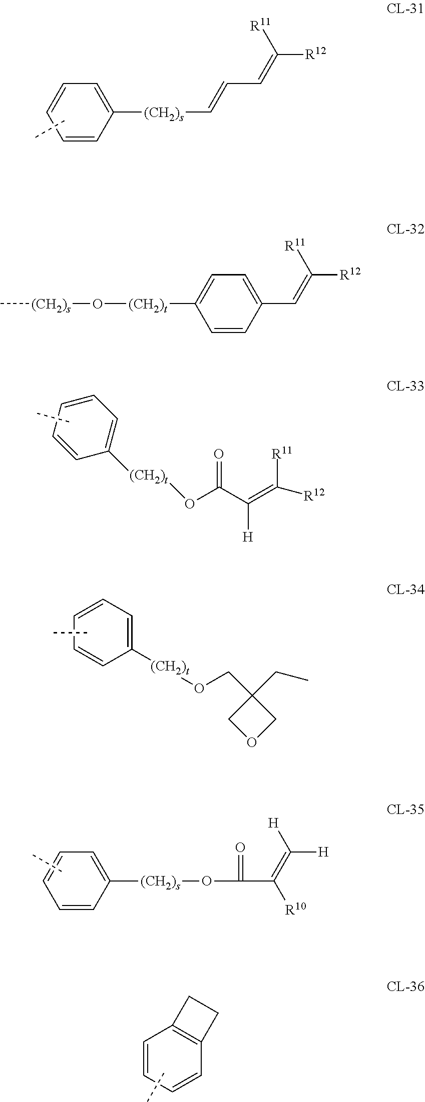
wherein: the dotted line represents a bonded bond, R¹⁰ to R¹³ are identically defined as the above-mentioned R₁ in claim 1, Ar¹² is defined as the above-mentioned Ar₁ in claim 1; s, t at each occurrence are integers >0.

8. The compound according to claim 4, wherein at least one of the cross-linkable group is selected from the following structures: 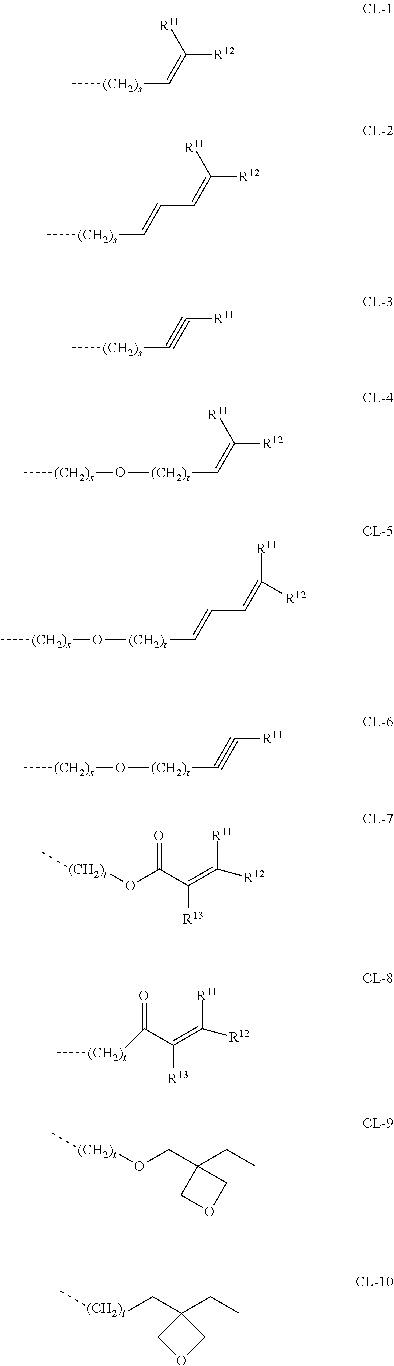 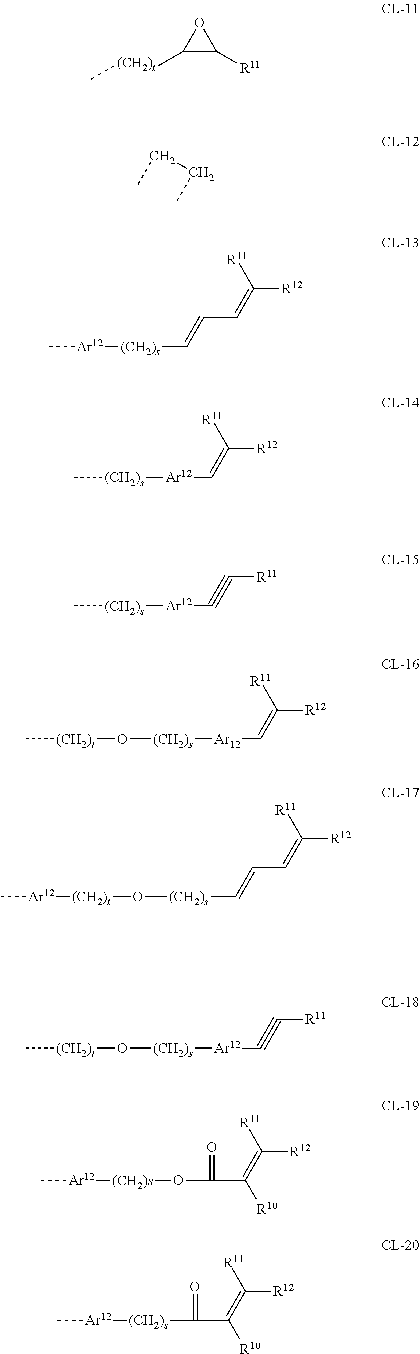 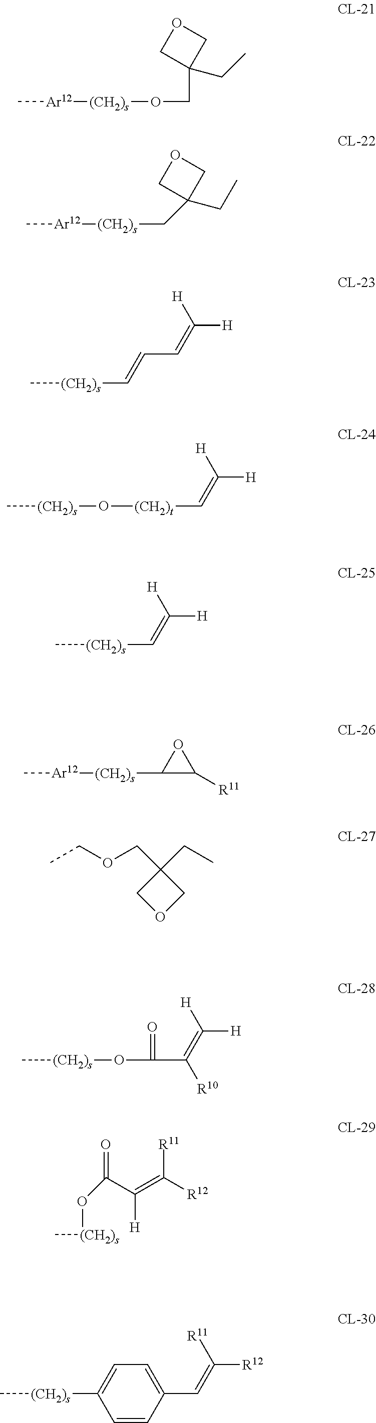 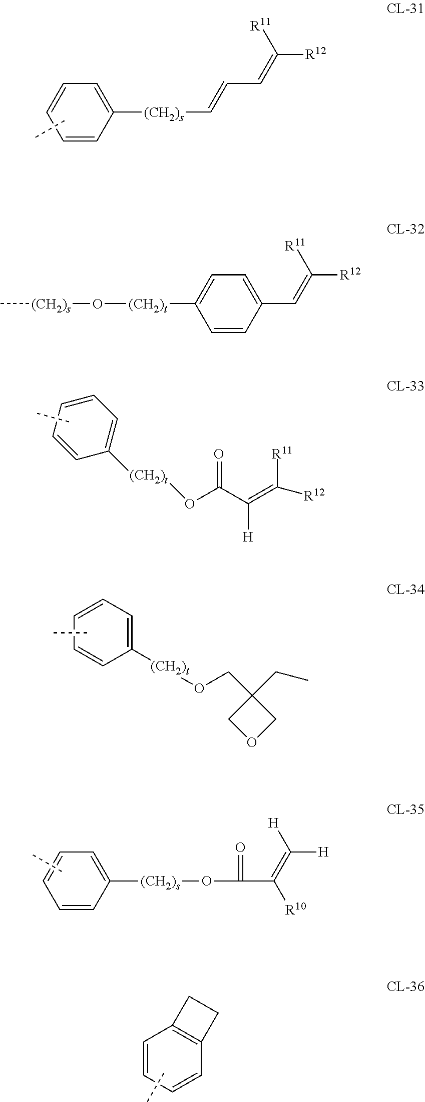
wherein: the dotted line represents a bonded bond, R¹⁰ to R¹³ are identically defined as the above-mentioned R₁ in claim 1, Ar¹² is defined as the above-mentioned Ar₁ in claim 1; s, t at each occurrence are integers >0.

9. A formulation, comprising at least one compound according to claim 1, at least one organic solvent, and/or an organic resin.

10. The formulation according to claim 9, wherein at least one of the organic solvent is selected from aromatic, heteroaromatic, esters, aromatic ketones, aromatic ethers, aliphatic ketones, aliphatic ethers, alicyclic or olefin compounds, borates, phosphorates, or mixtures of two or more of them.

11. The formulation according to claim 9, wherein the organic resin is a thermosetting resin or a UV curable resin.

12. The formulation according to claim 9, wherein the formulation further comprises an emitter E1, the emission spectrum of the compound is on the short wavelength side of the absorption spectrum of the emitter E1, and at least partially overlaps with the absorption spectrum of the emitter E1; and a full width at half maximum (FWHM) of the emission spectrum of the emitter E1 ≤55 nm.

13. The formulation according to claim 10, wherein the formulation further comprises an emitter E1, the emission spectrum of the compound is on the short wavelength side of the absorption spectrum of the emitter E1, and at least partially overlaps with the absorption spectrum of the emitter E1; and a FWHM of the emission spectrum of the emitter E1 ≤55 nm.

14. The formulation according to claim 11, wherein the formulation further comprises an emitter E1, the emission spectrum of the compound is on the short wavelength side of the absorption spectrum of the emitter E1, and at least partially overlaps with the absorption spectrum of the emitter E1; and a FWHM of the emission spectrum of the emitter E1 ≤55 nm.

15. An organic light-emitting device, comprising a substrate, a first electrode, an organic light-emitting layer, a second electrode, a color conversion layer, and an encapsulation layer in sequence from bottom to top, wherein the second electrode is at least partially transparent, the color conversion layer comprises the compound according to of claim 1 and an emitter E2; the color conversion layer at least partially absorbs the light emitted by the organic light-emitting layer through the second electrode; the emission spectrum of the compound is on the short wavelength side of the absorption spectrum of the emitter E2, and at least partially overlaps with the absorption spectrum of the emitter E2; and a FWHM of the emission spectrum of the emitter E2 ≤55 nm.
